(19) United States
(12) Patent Application Publication
(10) Pub. No.: US 2023/0218728 A1
(43) Pub. Date: Jul. 13, 2023

(54) TREATMENT OF POST-OPERATIVE SURGICAL PAIN

(71) Applicant: IPSEN BIOPHARM LIMITED, Wrexham (GB)

(72) Inventors: Steve EVANS, Wrexham (GB); Mikhail KALINICHEV, Wrexham (GB); Laurent PONS, Wrexham (GB); Sylvie CORNET, Wrexham (GB); Stephane LEZMI, Wrexham (GB)

(21) Appl. No.: 18/003,489

(22) PCT Filed: Jul. 16, 2021
(86) PCT No.: PCT/GB2021/051838

§ 371 Date: Dec. 27, 2022

(30) Foreign Application Priority Data
Jul. 17, 2020 (GB) 2011055.7

(51) Int. Cl. A61K 38/48; A61P 29/02; A61P 25/22

(52) U.S. Cl. CPC A61K 38/4893; A61P 29/02; A61P 25/22

(57) ABSTRACT

The present invention relates to a clostridial neurotoxin for use in treating post-operative surgical pain in a patient, said method comprising administering to a patient a clostridial neurotoxin more than 5 days prior to surgery and wherein the clostridial neurotoxin is administered: i) intradermally; or ii) intrathecally.

Also provided are corresponding methods of treatment and administration dosages of a clostridial neurotoxin.

### FIELD OF THE INVENTION

The present invention relates to the treatment of surgical pain resulting from surgery (e.g. post-operative surgical pain) and/or surgery-induced anxiety (e.g. post-operative anxiety). In more detail, the invention provides methods of treatment comprising administration of clostridial neurotoxins and more particularly, to a method of treating post-operative surgical pain and anxiety using botulinum neurotoxins.

### BACKGROUND

Post-operative surgical pain is an unpleasant sensation that results from a surgical procedure. Post-operative surgical pain may be caused by damage to tissue by surgical intervention, the surgical procedure itself, the closing of the wound, and any force that is applied during the surgical procedure. Surgical pain after surgery (e.g. post-operative surgical pain) can also stem from factors that accompany surgery. For example, a patient may suffer back pain due to the way the patient was positioned on the surgical table, or chest pain may be due to surgical intervention in the chest area. Throat pain may also occur after general anesthesia because the insertion of the breathing tube can cause irritation. However, most common is post-operative surgical pain caused by cutting into the skin and muscle from surgical intervention.

For example, the surgical intervention (or more particularly, surgical incision) may represent a ‘noxious stimulus’ causing pain. Noxious stimuli, stimuli which can elicit tissue damage, can activate the release of neurotransmitters from nociceptive afferent terminals and the release of neuropeptides such as Substance P and Calcitonin gene related peptide (CGRP) from sensory terminals. The noxious information is then transduced from the peripheral nervous system to the central nervous system, where pain is perceived by the individual.

Post-operative surgical pain can be caused by the combination of inflammation and neural tissue damage at a site of surgical intervention. Any inflammation and/or neural tissue damage is in addition to post-operative surgical pain. For example, degranulation of activated mast cells in response to tissue injury can result in the release of various substances including proteases, cytokines and serotonin. These substances can sensitize (activate at a lower threshold) primary afferent neurons to produce pain hypersensitivity. As tissue is extensively innervated, any region of the body is susceptible to nerve damage from surgery.

Post-operative anxiety may entail patients experiencing physical symptoms and behavioural changes including but not limited to fatigue, difficulties in concentrating and sleeping and muscle tension. Furthermore, the patient may experience emotional symptoms of anxiety, including restlessness, irritability, difficulties in controlling fear or worry, dread and panic. Post-operative anxiety may be caused by the effects of anesthesia, surgery itself, post-operative surgical pain and/or stress from the hospital environment. For example, surgery patients are often under considerable levels of mental, physical, and emotional stress both before (e.g. stress due to anticipation of surgery) and after surgery, with such stress manifesting in symptoms of anxiety.

Existing methods for the treatment of surgical (e.g. post-operative) pain typically target neurotransmitters and peptides, and include non-steroidal anti-inflammatory drugs (NSAIDS), opioids, local anesthetic blocks or their use in combination. However, these methods of treatment produce a variety of side effects, and, notably, often induce dependency (e.g. addiction). Additionally, these methods of treatment only provide acute post-operative surgical pain relief, for example providing surgical pain relief for only a short period of time after administration, thus necessitating the need for continuous/repeat administration (exacerbating the problem of patient dependency on/addiction to analgesics). Without effectively managing acute post-operative surgical pain, this can increase the chances of the patient developing chronic post-operative surgical pain. Such methods of pain management (requiring continuous administration of the drug) also often result in drug resistance. Further problems associated with previous methods for managing post-operative surgical pain include the need for high doses of analgesic drug administration to provide an analgesic effect, with adverse side effects often increasing congruent with the dose.

Thus, current methods of treating surgical pain are not appropriate (e.g. sufficient) for managing/alleviating post-operative surgical pain, especially moderate to severe post-operative surgical pain and neither do they provide appropriate management of post-operative anxiety experienced by the patient (the latter generally requiring alternative/additional medication). Accordingly, there is an increasing need for alternative/improved methods for treating post-operative surgical pain and/or post-operative anxiety.

The present invention addresses one or more of these problems by providing a method for the long-lasting treatment of post-operative surgical pain/anxiety (including reduced propensity for chronic post-operative surgical pain), for example even after a single (e.g. acute) administration. Preferred aspects of the invention are predicated on the surprising observation that initiating treatment (administration) prior to subjecting a patient to surgery allows for effective post-operative surgical pain or post-operative anxiety management as the patient emerges from surgery (advantageously mitigating post-operative surgical pain or post-operative anxiety that would otherwise be perceived as the effect of general/local anaesthetic abates), and particular pre-surgery administration timepoints that are uniquely suited to clostridial neurotoxin-based treatment are provided.

### SUMMARY OF THE INVENTION

In more detail, the present invention is predicated on the surprising finding that administration of a clostridial neurotoxin (such as a botulinum neurotoxin) pre-surgery by intradermal or intrathecal administration, more than 5 days prior to surgery treats post-operative surgical pain and reduces or suppresses post-operative anxiety. This was completely unexpected as prior art methods reported optimal analgesic activity when clostridial neurotoxin was administered closer to the time of surgery and by alternative administration routes.

Advantageously, the inventors have demonstrated that, by administering the clostridial neurotoxin more than 5 days prior to surgery by intradermal or intrathecal administration, the clostridial neurotoxin is effective at treating post-operative surgical pain at a time point as early as one hour post-surgery, and continues to manage/treat post-operative surgical pain for several days (even weeks) post-surgery without the need for continuous administration and without the side effects associated with the traditional analgesic/anaesthetic drugs. As such, the clostridial neurotoxin can provide post-operative surgical pain relief as the effects of any ‘general’ or ‘local’ anaesthetic (used during surgery) wears off. In other words, pre-surgery (>5 days pre-surgery) administration advantageously allows the analgesic effects of the clostridial neurotoxin to arise (e.g. reach maximal efficacy/effect) at a timepoint when the patient would otherwise begin to perceive post-operative surgical pain or post-operative anxiety as the effect of the principal anaesthetic/analgesic used during surgery tapers off. Therefore, post-operative surgical pain can be treated before it arises, preventing any associated (potentially significant) discomfort and distress in the patient. As described in more detail below, such early management of post-operative surgical pain (e.g. acute moderate to severe post-operative surgical pain) may advantageously mitigate the onset of chronic post-operative surgical pain.

This contrasts with treatment observed when the clostridial neurotoxin is administered closer to the time of surgery (or peri-operatively), wherein a distinct lag phase, or ‘activation period’, is observed post-surgery until any surgical pain-treatment effect of the clostridial neurotoxin is provided.

A yet further surprising observation by the inventors is that a clostridial neurotoxin is a particularly efficacious (e.g. rapid) post-operative surgical pain/post-operative anxiety treatment where the clostridial neurotoxin is administered intradermally. Indeed, the inventors observed that intradermal administration may provide for increased post-operative surgical pain and/or post-operative anxiety relief when compared with alternative administration routes such as subcutaneous administration and intramuscular administration. This was totally unexpected, as clostridial neurotoxins are regularly administered by such alternative administration routes (e.g. subcutaneous/intramuscular administration) in other indications without any apparent disadvantages in terms of efficacy.

Another advantageous finding by the inventors is that a clostridial neurotoxin is able to exert an effect at a site distal to the site of administration. For example, following administration of a clostridial neurotoxin at or proximal to a site of surgical intervention, the inventors observed SNARE protein cleavage (e.g. SNAP-25 protein cleavage) at the spinal cord (and minimal or no SNARE protein cleavage at or proximal to the site of surgical intervention), suggesting that the clostridial neurotoxin travels by retrograde transport from its site of administration to the spinal cord. This allows a clostridial neurotoxin to be administered at a site away from a(ny) site of injury (e.g. the site of surgical intervention) that may cause discomfort to a patient and thus minimise any further pain perceived by the patient.

### DETAILED DESCRIPTION

In one aspect the invention provides a clostridial neurotoxin for use in treating post-operative surgical pain in a patient, said method comprising administering to a patient a clostridial neurotoxin more than 5 days prior to surgery and wherein the clostridial neurotoxin is administered:
i) intradermally; or
ii) intrathecally.

In other words, an aspect of the invention provides a method for treating post-operative surgical pain in a patient, said method comprising administering to a patient a clostridial neurotoxin more than 5 days prior to surgery and wherein the clostridial neurotoxin is administered:
i) intradermally; or
ii) intrathecally.

In one aspect the invention provides a clostridial neurotoxin for use in treating post-operative pain, said method comprising administering (e.g. intradermally) to a patient a clostridial neurotoxin more than 5 days prior to surgery. In other words, an aspect of the invention provides a method of treating post-operative pain, said method comprising administering (e.g. intradermally) to a patient a clostridial neurotoxin more than 5 days prior to surgery.

In a related observation, it has been found that pre-administration (before surgery) of a clostridial neurotoxin reduces or suppresses/prevents (e.g. completely prevents) the onset of anxiety in a patient post-surgery (post-operative anxiety). Therefore, a yet further surprising technical effect provided by the invention is an anxiolytic effect achievable due to pre-operative administration with a clostridial neurotoxin.

Thus, another aspect of the invention provides a clostridial neurotoxin for use in reducing or suppressing post-operative anxiety, said method comprising administering to a patient a clostridial neurotoxin prior to surgery, wherein the clostridial neurotoxin is administered:
i) intradermally; or
ii) intrathecally.

In other words, an aspect of the invention provides a method for reducing or suppressing post-operative anxiety, said method comprising administering to a patient a clostridial neurotoxin prior to surgery, wherein the clostridial neurotoxin is administered:
i) intradermally; or
ii) intrathecally.

Preferably, a method for reducing or suppressing post-operative anxiety comprises administering the clostridial neurotoxin 5 or more days prior to surgery; for example, the clostridial neurotoxin may be administered more than 5 days prior to surgery.

Another aspect of the invention provides a clostridial neurotoxin for use in reducing or suppressing post-operative anxiety, said method comprising administering (e.g. intradermally) to a patient a clostridial neurotoxin prior to surgery (such as 5 or more days prior to surgery).

In other words, an aspect of the invention provides a method of reducing or suppressing post-operative anxiety, said method comprising administering (e.g. intradermally) to a patient a clostridial neurotoxin prior to surgery.

Preferably, a method for reducing or suppressing post-operative anxiety comprises administering the clostridial neurotoxin 5 or more days prior to surgery; for example, the clostridial neurotoxin may be administered more than 5 days prior to surgery.

Preferably, the clostridial neurotoxin may be administered intradermally.

Additionally or alternatively, the clostridial neurotoxin may be administered intrathecally (e.g. by intrathecal administration/injection).

Various additional (optional) embodiments of the invention will now be described. It should be noted that each of the following embodiments may apply to any method, or clostridial neurotoxin for use described herein.

In one embodiment, the administration of the clostridial neurotoxin does not include intramuscular administration.

In one embodiment, the clostridial neurotoxin may be administered 5-50 days prior to surgery, for example 6-50 days prior to surgery, optionally 5-40 days prior to surgery. For example, the clostridial neurotoxin may be administered 5-30 days prior to surgery; preferably 5-20 days prior to surgery; more preferably 5-15 days prior to surgery.

In one embodiment, the clostridial neurotoxin may be administered >5 days prior to surgery (preferably in a method for treating post-operative surgical pain described herein), optionally in a single administration step. For example, the clostridial neurotoxin may be administered 10-20 days prior to surgery; 14-16 days prior to surgery (preferably in a method for treating post-operative surgical pain described herein), optionally in a single administration step.

The clostridial neurotoxin may be administered 15 or more days prior to surgery, preferably about 15 days prior to surgery.

In a preferred embodiment, the clostridial neurotoxin may be administered >5 days to ≤15 days prior to surgery (preferably in a method for treating post-operative surgical pain described herein), optionally in a single administration step.

In one embodiment, the clostridial neurotoxin is administered 5 or more days prior to surgery (preferably in a method for treating post-operative surgical pain described herein).

In one embodiment, the clostridial neurotoxin is administered 12 or more days prior to surgery (preferably in a method for treating post-operative surgical pain described herein).

In one embodiment, the clostridial neurotoxin is administered intradermally 15 or more days prior to surgery. In a preferred embodiment, the clostridial neurotoxin is administered intradermally about 15 days prior to surgery.

In one embodiment, the clostridial neurotoxin is administered intrathecally 15 or more days prior to surgery. In a preferred embodiment, the clostridial neurotoxin is administered intrathecally about 15 days prior to surgery.

The clostridial neurotoxin treats post-operative surgical pain through the provision of an analgesic effect. Thus, the term “treat” or “treating” as used herein is intended to encompass analgesic treatment. The term “treat” or “treating” encompasses treating post-operative surgical pain such that the patient no longer perceives surgical pain (or perceives less surgical pain compared to a control patient not treated with the clostridial neurotoxin).

Similarly, the clostridial neurotoxin suppresses post-operative anxiety through the provision of an anxiolytic effect. Thus, the term “suppress” or “suppressing” encompasses suppression of post-operative anxiety (e.g. symptoms thereof) in a patient via an anxiolytic effect provided by administration of a clostridial neurotoxin. The suppression may be provided concomitantly with, and for example as a result of, post-operative surgical pain treatment. Thus, without being bound to any theories, the clostridial neurotoxin may provide an anxiolytic effect by means of the clostridial neurotoxin's analgesic effect. The clostridial neurotoxin may suppress symptoms of post-operative anxiety which are associated with (or arise from) effects of anaesthesia employed for surgery, surgery itself, post-operative surgical pain and/or stress (e.g. from the hospital environment).

Therefore, a clostridial neurotoxin may be administered to a subject in a therapeutically effective amount or a prophylactically effective amount (preferably prophylactically effective amount). A “therapeutically effective amount” is any amount of the clostridial neurotoxin that, when administered alone or in combination to a subject for treating post-operative surgical pain and/or post-operative anxiety is sufficient to effect such treatment of post-operative surgical pain and/or post-operative anxiety. A “prophylactically effective amount” is any amount of the clostridial neurotoxin that, when administered alone or in combination to a subject inhibits or delays the onset of post-operative surgical pain and/or post-operative anxiety. In some embodiments, the prophylactically effective amount prevents the onset of post-operative anxiety entirely. “Inhibiting” the onset means either lessening the likelihood of post-operative surgical pain and/or post-operative anxiety, or preventing the onset entirely.

Preferably, a therapeutically and/or prophylactically effective amount is an amount which does not lead to muscle paralysis. The term “muscle paralysis” preferably refers to long-term muscle paralysis, since transient muscle paralysis may occur for a short period following administration.

The terms “subject”, “individual” and “patient” may be used interchangeably herein to refer to a mammalian subject. In one embodiment the “subject” is a human, a companion animal (e.g. a pet such as a dog, cat, and/or rabbit), livestock (e.g. a pig, sheep, cattle, and/or a goat), and/or a horse. In a preferable embodiment, the subject (patient) is a human.

The present invention relates specifically to post-operative surgical pain, which is distinct from other types of pain such as inflammatory pain and neuropathic pain. In this regard, inflammatory pain typically arises from an infection, irritants or an overactivated immune response and neuropathic pain typically arises from a nervous system disorder/syndrome. In contrast, the present invention does not relate to pain arising from these stimuli.

In one embodiment administration of a clostridial neurotoxin treats post-operative surgical pain preferentially to inflammatory pain. In one embodiment administration of a clostridial neurotoxin treats minimal to no inflammatory pain. In one embodiment administration of a clostridial neurotoxin treats post-operative surgical pain and minimal to no inflammatory pain.

Reference to “surgical intervention” means a medical procedure involving the treatment of an injury or disease in a subject comprising subjecting a part of the body to an incision (optionally removing or repairing a damaged part of the body). Although the level of invasiveness (e.g. level of surgical incision required) may vary amongst surgery types, surgery having a level of invasiveness that causes post-operative surgical pain and/or post-operative anxiety in the subject once surgery is complete is intended to be encompassed. Post-operative surgical pain is typically caused by a surgical incision that cuts through the skin and/or fascia and/or muscle and/or bone and/or organ in a patient. Thus, surgical pain is typically experienced at/or proximal to the site of surgical intervention.

The surgical intervention may comprise an incision to skin and/or fascia and/or muscle. Preferably, the surgical intervention comprises of an incision to the skin.

The surgical intervention is not limited to that which may be carried out by a physician, but also includes for example dental surgical intervention. Non-limiting examples of surgical intervention include appendectomy, breast biopsy, breast augmentation or reduction, facelift, cholecystectomy, coronary artery bypass, debridement (e.g. of a wound, a burn, or infection), skin graft, organ transplant and tonsillectomy.

Preferably, “post-operative” may refer to a time period beginning at most one day subsequent to surgery (e.g. post-surgery). In other words, the term “post-operative” may refer to a time period beginning not greater than one day post-surgery. For example, the term “post-operative” may refer to a time point beginning 1-20 hours post-surgery; optionally 2-15 hours post-surgery; optionally 5-10 hours post-surgery. Such time may represent a time period beginning at the chronological interface at which the analgesic effects from a surgical anaesthetic administered to a patient diminish (e.g. taper) and thus the patient begins to perceive surgical pain.

Furthermore, the term “post-operative” may be used interchangeably with the term “post-surgical”, as ‘operative’ is used in the sense of ‘surgery’ herein.

Similarly, the term “post-operative surgical pain” may refer to surgical pain that is perceived (or more particularly, begins to be perceived) for a time period beginning at most one day subsequent to surgery (e.g. post-surgery). In other words, the term “post-operative surgical pain” may refer to surgical pain that is perceived by a patient for a time period beginning not greater than one day post-surgery. For example, the term “post-operative surgical pain” may refer to pain that is perceived for a time period beginning 1-20 hours post-surgery; optionally 2-15 hours post-surgery; optionally 5-10 hours post-surgery.

Said time period may be 1-50 weeks; for example 5-45 weeks, 10-40 weeks or 10-35 weeks post-surgery.

This contrasts with the term “peri-operative”, which may refer, for example, to a time period at or around the time that a patient is undergoing surgery (e.g. the time when the patient is in the operating theatre), suitably a period beginning at least 1 hour pre-surgery and/or ending less than 1 hour post-surgery.

The post-surgery treatment of the present invention may be combined with a peri-operative and/or post-operative treatment strategy to improve efficacy and preferably increase duration or post-operative surgical pain suppression (e.g. in patients at high risk of developing surgical pain).

In one embodiment, a method of the invention may comprise administering to a patient a further analgesic peri-operatively and/or post-operatively (preferably post-operatively). In other words, a further analgesic may be administered during surgery (e.g. during a period beginning at least 1 hour pre-surgery and/or ending less than 1 hour post-surgery). In one embodiment, a further analgesic may be administered subsequent to surgery (e.g. at 10, 20, 40, or 50 weeks post-surgery).

The post-operative surgical pain may be pain caused by the release of neurotransmitters from nociceptive afferent terminals and/or the release of neuropeptides such as Substance P and Calcitonin gene related peptide (CGRP) from sensory terminals, for example induced by a noxious stimulus of surgery (preferably a surgical incision). For example, noxious information (resulting from noxious stimuli) may then be transduced from the peripheral nervous system to the central nervous system, where surgical pain is perceived by the patient.

In one embodiment, the clostridial neurotoxin treats surgical pain through the inhibition of exocytosis of pain neuromodulators such as Substance P and CGRP.

Post-operative surgical pain may be caused by inflammation or neural tissue damage at a site of surgical intervention, or a combination thereof. Any inflammation and/or neural tissue damage is in addition to post-operative surgical pain. For example, degranulation of activated mast cells in response to tissue injury may result in the release of various substances including proteases, cytokines, and serotonin. These substances can sensitize (activate at a lower threshold) primary afferent neurons to produce pain hypersensitivity. As tissue is extensively innervated, any region of the body may be susceptible to nerve damage from surgery.

In other words, the pain may be nociceptive pain, for example where post-operative surgical pain arises from tissue damage and is perceived by the activation of nociceptors (pain receptors) in response to noxious stimuli.

In one embodiment, the post-operative surgical pain may be neuropathic pain (e.g. pain caused by damage or disease affecting the somatosensory nervous system). For example, the post-operative surgical pain may be peripheral neuropathy (also known as peripheral pain), for example where pain results from damage to nerves outside of the brain and spinal cord (peripheral nerves).

Post-operative surgical pain may manifest as other types of pain such as allodynia. Allodynia means “other pain.” It is a pain that results from a stimulus that is not normally painful. A sufferer of ‘tactile’ allodynia (aka static tactile allodynia or mechanical allodynia) may experience pain to touch, such as with resting the (body) site of surgical incision on a bed, or with wearing clothing which contacts said site. Thus, allodynia is considered “pain due to a stimulus that does not usually provoke pain”, as opposed to hyperalgesia (increased pain from a stimulus that does usually provoke pain).

The post-operative surgical pain may preferably be acute post-operative surgical pain; for example, a type of surgical pain that may last less than 3 months (post-surgery).

In one embodiment, the post-operative surgical pain is chronic post-operative surgical pain; for example, a type of surgical pain that may last longer than three months (post-surgery) and may continue to be perceived after tissue damage (e.g. due to surgical incision) has healed.

In more detail, the term “chronic post-operative surgical pain” as used herein preferably refers to pain persisting for longer than 3 months beyond resolution of the underlying insult (e.g. damage to muscles from surgical incision), for example pain persisting beyond 3 months post-surgery. “Chronic post-operative surgical pain” may, for example, develop from insufficient (or lack of) treatment of acute post-operative surgical pain (e.g. a type of surgical pain that typically lasts for less than 3 months). Poor management of post-operative “acute surgical pain” may increase the chance of such acute surgical pain becoming chronic post-operative surgical pain.

Thus, advantageously, by managing acute post-operative surgical pain at an early stage (advantageously due to the pre-surgery administration providing for analgesic efficacy shortly after surgery), the prevent invention mitigates the occurrence of chronic post-operative surgical pain.

Chronic post-operative surgical pain may be perceived at or around a scar (a scar formed at the site of surgical incision). In a preferable embodiment, the chronic post-operative surgical pain is chronic scar pain. The term “chronic scar pain” refers to pain that develops as a result from tissue scarring. “Chronic scar pain” may develop from damage to skin and/or muscle tissue and/or nerve tissue and the regeneration of nerves.

In a preferable embodiment, post-operative surgical pain is surgical pain that is perceived at the site of surgical intervention and/or surgical pain perceived at a site proximal to the site of surgical intervention, preferably wherein the surgical pain is perceived within tissue (e.g. skin, muscle) that has been damaged through surgical intervention. Post-operative surgical pain may also be surgical pain perceived at a site of tissue/organ internal to the body that has been subjected to biopsy.

Preferably, administration of a clostridial neurotoxin reduces a level of surgical pain perception by the patient post-surgery. For example, the level of surgical pain perception by the patient may be reduced post-surgery compared with a level of surgical pain perception in a (control) patient that was not administered a clostridial neurotoxin prior to surgery (e.g. 5 or more days prior to surgery).

In one embodiment, the post-operative surgical pain perception by the patient is reduced within 24 hours post-surgery. In other words, administration of a clostridial neurotoxin may reduce the patient's post-operative surgical pain perception within 24 hours post-surgery. For example, administration of a clostridial neurotoxin may reduce the patient's post-operative pain perception within 6 hours post-surgery, preferably within 1 hour post-surgery.

The patient's post-operative surgical pain perception may be reduced for at least 3 days, post-surgery, at least 6 days post-surgery or at least 9 days post-surgery; for example, for at least 15 days post-surgery; in another example, for at least 30 days post-surgery. In a preferable embodiment, the patient's post-operative surgical pain may be reduced for up to 3 months post-surgery (inclusive).

The reduced post-operative surgical pain perception by the patient may be maintained for at least 5 days post-surgery, at least 7 days post-surgery or at least 9 days post-surgery, preferably for at least 9 days post-surgery.

In one embodiment administration of the clostridial neurotoxin substantially reduces post-operative surgical pain perception by the patient and wherein the reduced post-operative surgical pain perception is maintained for 24 hours immediately following surgery. In another embodiment post-operative surgical pain perception by the patient is substantially reduced and maintained for 2 days immediately following surgery. In one embodiment substantially all reduced post-operative surgical pain perception is maintained for 3 days immediately following surgery. In one embodiment substantially all reduced post-operative surgical pain perception is maintained for 4 days immediately following surgery. In one embodiment substantially all reduced post-operative surgical pain perception is maintained for 5 days immediately following surgery. In one embodiment substantially all reduced post-operative surgical pain perception is maintained for 6 days immediately following surgery. In one embodiment substantially all reduced post-operative surgical pain perception is maintained for 7 days immediately following surgery. Preferably, substantially all reduced post-operative surgical pain perception is maintained for 8 days immediately following surgery.

In one embodiment the reduced level of pain perception observed at a defined time point (as aforementioned in the preceding paragraph) immediately following surgery is at least 50% of the maximum level of reduced pain perception observed at any time following administration of the clostridial neurotoxin. For example, the reduced level of pain perception observed at a defined time point (as aforementioned in the preceding paragraph) immediately following surgery is at least 55%, at least 60%, at least 65%, at least 70%, preferably at least 75% of the maximum level of reduced pain perception observed at any time following administration of the clostridial neurotoxin.

In more detail, reference to “reduced” (in terms of post-operative surgical pain) preferably means a lower level of surgical pain is perceived by the subject (e.g. patient) administered with clostridial neurotoxin when compared with a level of surgical pain received by a subject (that has likewise been subjected to surgery) administered no clostridial neurotoxin (or administered a placebo). For example, the level of surgical pain perception may be reduced by at least 15%, 25%, 35%, 45%, 55%, 65%, 75%, 85% or 95% post-administration of the clostridial neurotoxin, when compared with a subject (that has likewise been subjected to surgery) administered no clostridial neurotoxin (or administered a placebo). For example, the level of surgical pain perception may be reduced by at least 75%; preferably at least 85%; more preferably at least 95% post-administration of the clostridial neurotoxin, when compared with a subject (that has likewise been subjected to surgery) administered no clostridial neurotoxin (or administered a placebo).

A variety of means for assessing pain perception are known to those skilled in the art. For example, evaluation of mechanical allodynia (either static or dynamic) is routinely used in human pain studies as described in Pogatzki-Zahn et. al. (Pain Rep. 2017 March; 2(2): e588), incorporated herein by reference.

A suitable (albeit non-limiting example) method for assessing pain perception in a subject includes the following: Numerical Rating Scale (NRS) score; although the skilled person is aware of other methods which may be used additionally or alternatively such as sensory threshold, pain perception threshold, static mechanical allodynia, dynamic mechanical allodynia, temporal summation, pressure pain threshold, conditioned pain modulation, and temperature threshold.

Other non-limiting examples of pain perception measures include: change from baseline in SF-36 scores at each scheduled time point; amount of rescue medication taken during the study and time to first intake of rescue medication. These may be considered “exploratory” endpoints or pain perception assessment measures.

Thus, in a preferred embodiment, following the administration of clostridial neurotoxin, post-operative surgical pain perception may be assessed by one or more of: (a) a Numerical Rating Scale (NRS); (b) stimulus-evoked NRS; (c) temperature of the painful area; (d) size of the painful area; (e) time to onset of analgesic effect; (f) peak analgesic effect; (g) time to peak analgesic effect; (h) duration of analgesic effect; and (i) SF-36 quality of life.

The skilled person is aware of such methods for assessing pain perception. For convenience, further description of the Numerical Rating Score and Quality of Life questionnaire Short Form-36 are provided below.

Numerical Rating Scale (NRS): Typically surgical pain perception according to the present invention uses the Numerical Rating Scale (NRS). The NRS is an 11-point scale to assess subject surgical pain perception. Subjects are asked to give a number between 0 and 10 that fits best to their surgical pain intensity. Zero represents ‘no surgical pain at all’ whereas the upper limit, 10, represents ‘the worst surgical pain possible’.

The NRS can be used to assess numerous facets of surgical pain, including spontaneous average surgical pain, spontaneous worst surgical pain and spontaneous current surgical pain. Spontaneous average surgical pain is assessed by asking a subject to select a number that best describes the subject's average surgical pain (e.g. perceived surgical pain) over a period of time, for example at least 6 hours, 12 hours, 24 hours, or at least 48 hours. Spontaneous worst surgical pain is assessed by asking a subject to select a number that best describes the subject's surgical pain at its worst during a specified period, e.g. at least the previous 6 hours, 12 hours, 24 hours or previous 48 hours. Spontaneous current surgical pain is assessed by asking a subject to select a number that best describes how much surgical pain the subject is in at the time of assessment.

The NRS can also be used to assess a subject's surgical pain perception in response to a variety of different stimuli. To assess surgical pain perception in response to a stimulus, the subject will be submitted to stimuli of various nature applied to the painful area. Subjects will be asked what are their current NRS scores pre-dose and post-stimulus.

Examples of stimuli used include: (i) light touch (which can be assessed by measuring pain on the surface of the painful area on radial spokes following application of a von Frey filament as described herein); (ii) pressure (pressure pain threshold), which can be assessed by asking the subject to give a NRS score as increasing pressure is applied using a pressure algometer as described herein; and (iii) temperature (which can be assessed by asking the subject for an NRS score for warm, cold and hot stimulation using a thermode applied to the painful area, as described herein).

Preferably, administration of a clostridial neurotoxin described herein reduces the patient's NRS score post-surgery (e.g. from a rating of ≥7 to a rating of 56) when compared with an NRS score of a control patient that is not administered a clostridial neurotoxin.

Quality of Life questionnaire Short Form-36 (SF-36): The SF-36 quality of life questionnaire may be used to assess a subject's surgical pain perception. The SF-36 is a 36-item, subject-reported survey of subject health. The SF-36 consists of eight scaled scores (vitality, physical functioning, bodily pain, general health perceptions, physical role functioning, emotional role functioning, social role functioning and mental health). Each scale is directly transformed into a 0-100 scale on the assumption that each question carries equal weight. The higher the score recorded in the SF-36, the less disability.

Relevant parameters commonly tested in clinical trials for the treatment of surgical pain are known in the art and could be readily selected by one of ordinary skill in the art. Examples of such parameters include, but are not limited to NRS; stimulus-evoked NRS; temperature of the painful area; size of the painful area; time to onset of analgesic effect; peak analgesic effect; time to peak analgesic effect; duration of analgesic effect; and/or SF-36 quality of life as described herein. Methods for assessing these parameters are also known in the art and can be carried out by one of ordinary skill using routine methods and procedures.

Preferably, administration of a clostridial neurotoxin described herein increases the patient's SF-36 score post-surgery (e.g. from a score of ≤50 to a score of ≥50) when compared with an SF-36 score of a control patient that is not administered a clostridial neurotoxin. Turning now to “post-operative anxiety”, reference to the same (i.e, post-operative anxiety) may refer to a condition in which a patient experiences physical symptoms and behavioural changes including but not limited to fatigue, difficulties in concentrating and sleeping and muscle tension. Furthermore, the patient may experience emotional symptoms of anxiety, including restlessness, irritability, difficulties in controlling fear or worry, dread and panic. Post-operative anxiety may be caused by the effects of anesthesia, surgery itself, post-operative surgical pain and stress from the hospital environment.

Thus, in one embodiment, post-operative anxiety is caused by post-operative surgical pain.

The post-operative anxiety may be defined as a panic disorder, a phobia, a post-traumatic stress disorder, a social anxiety disorder (social phobia) or a generalised anxiety disorder (GAD) (e.g. a long-term condition that causes you to feel anxious about a wide range of situations and issues, rather than one specific event).

In one embodiment, the clostridial neurotoxin for use in reducing or suppressing post-operative anxiety or the method comprising administering to a patient a clostridial neurotoxin prior to surgery is administered 5 or more days prior to surgery, preferably wherein the clostridial neurotoxin is administered more than 5 days prior to surgery.

In one embodiment administration of the clostridial neurotoxin substantially reduces post-operative anxiety perception by the patient and wherein said reduced post-operative anxiety perception is maintained for 24 hours immediately following surgery. In one embodiment substantially all reduced post-operative anxiety perception is maintained for 2 days immediately following surgery. In one embodiment substantially all reduced post-operative anxiety perception is maintained for 3 days immediately following surgery. In one embodiment substantially all reduced post-operative anxiety perception is maintained for 4 days immediately following surgery. In one embodiment substantially all reduced post-operative anxiety perception is maintained for 5 days immediately following surgery. In one embodiment substantially all reduced post-operative anxiety perception is maintained for 6 days immediately following surgery. In one embodiment substantially all reduced post-operative anxiety perception is maintained for 7 days immediately following surgery. In one embodiment substantially all reduced post-operative anxiety perception is maintained for 8 days immediately following surgery. Preferably, substantially all reduced post-operative anxiety perception is maintained for 9 days immediately following surgery.

Preferably, administration of a clostridial neurotoxin described herein reduces a symptom of anxiety post-surgery, when compared with a symptom of a control patient that is not administered a clostridial neurotoxin (e.g. by 30%, 50%, 75% or 95%). Examples of symptoms of post-operative anxiety include restlessness, irritability, difficulties in controlling fear or worry, dread and panic.

In more detail, reference to “reduced” (in terms of post-operative anxiety) preferably means a lower level of anxiety is perceived by the subject (e.g. patient) administered with clostridial neurotoxin when compared with a level of anxiety perceived by a subject (that has likewise been subjected to surgery) administered no clostridial neurotoxin (or administered a placebo). For example, the level of anxiety perception may be reduced by at least 15%, 25%, 35%, 45%, 55%, 65%, 75%, 85% or 95% post-administration of the clostridial neurotoxin, when compared with a subject (that has likewise been subjected to surgery) administered no clostridial neurotoxin (or administered a placebo). For example, the level of anxiety perception may be reduced by at least 75%; preferably at least 85%; more preferably at least 95% post-administration of the clostridial neurotoxin, when compared with a subject (that has likewise been subjected to surgery) administered no clostridial neurotoxin (or administered a placebo).

In one embodiment, post-operative anxiety experienced by the patient is suppressed within 24 hours post-surgery. In other words, administration of a clostridial neurotoxin may reduce or suppress a patient's post-operative anxiety within 24 hours post-surgery. For example, the patient's post-operative anxiety may be reduced or suppressed within 2 hours post-surgery, within 4 hours post-surgery or within 24 hours post-surgery; preferably within 4 hours post-surgery.

The inventors have demonstrated that a clostridial neurotoxin may be administered to both treat post-surgical pain as well as suppress post-operative anxiety. Thus, in one embodiment the clostridial neurotoxin treats post-operative pain and reduces or suppresses post-operative anxiety.

Taken together, the invention advantageously increases a patient's overall “post-operative wellness”, through reducing the level of both surgical pain and anxiety otherwise perceived following the surgery/operation and thus improving the patient's quality of life.

Thus, in one embodiment the administration of a clostridial neurotoxin promotes post-operative wellness.

Further details of the clostridial neurotoxins embraced by the invention are provided below, together with technological background information.

Bacteria in the genus Clostridia produce highly potent and specific protein toxins, which can poison neurons and other cells to which they are delivered. Examples of such clostridial toxins include the neurotoxins produced by C. tetani (TeNT) and by C. botulinum (BoNT) serotypes A-G, as well as those produced by C. baratii and C. butyricum.

Clostridial neurotoxins (for example, in nature) cause muscle paralysis by inhibiting cholinergic transmission in the peripheral nervous system, in particular at the neuromuscular junction, and can thus be lethal. In nature, clostridial neurotoxins are synthesised as a single-chain polypeptide that is modified post-translationally by a proteolytic cleavage event to form two polypeptide chains joined together by a disulphide bond. Cleavage occurs at a specific cleavage site, often referred to as the activation site, which is located between the cysteine residues that provide the inter-chain disulphide bond. It is this di-chain form that is the active form of the toxin. The two chains are termed the heavy chain (H-chain), which has a molecular mass of approximately 100 kDa, and the light chain (L-chain), which has a molecular mass of approximately 50 kDa. The H-chain comprises an N-terminal translocation component (HN domain) and a C-terminal targeting component (HC domain). The cleavage site is located between the L-chain and the HN domain.

The mode of action of clostridial neurotoxins relies on five distinct steps: (1) binding of the HC domain to the cell membrane of its target neuron, followed by (2) internalisation of the bound toxin into the cell via an endosome, (3) translocation of the L-chain by the HN domain across the endosomal membrane and into the cytosol, (4) proteolytic cleavage of intracellular transport proteins known as SNARE proteins by the L-chain which provides a non-cytotoxic protease function, and (5) inhibition of cellular secretion from the target cell.

Non-cytotoxic proteases act by proteolytically cleaving intracellular transport proteins known as SNARE proteins (e.g. SNAP-25, VAMP, or Syntaxin)—see Gerald K (2002) “Cell and Molecular Biology” (4th edition) John Wiley & Sons, Inc. The acronym SNARE derives from the term Soluble NSF Attachment Receptor, where NSF means N-ethylmaleimide-Sensitive Factor. SNARE proteins are integral to intracellular vesicle fusion, and thus to secretion of molecules via vesicle transport from a cell. The protease function is a zinc-dependent endopeptidase activity and exhibits a high substrate specificity for SNARE proteins. Accordingly, once delivered to a desired target cell, the non-cytotoxic protease is capable of inhibiting cellular secretion from the target cell. The L-chain proteases of clostridial neurotoxins are non-cytotoxic proteases that cleave SNARE proteins.

Thanks to their unique properties, Clostridial neurotoxins, such as botulinum toxin, have been successfully employed in a wide range of therapeutic applications, in particular for motor and autonomic disorders, to restore for example the activity of hyperactive nerve endings to normal levels. At least seven antigenically distinct BoNTs serotypes have been described so far, namely BoNT/A, BoNT/B, BoNT/C, BoNT/D, BoNT/E, BoNT/F, BoNT/G (Rossetto, O. et al., “Botulinum neurotoxins: genetic, structural and mechanistic insights.” Nature Reviews Microbiology 12.8 (2014): 535-549).

Despite this diversity, BoNT/A remains the serotype of choice in therapy, with three commonly available commercial preparations (Botox®, Dysport® and Xeomin®), while only one BoNT/B product is available on the market (Neurobloc®/Myobloc®). To this day, these BoNT/A and BoNT/B products, which are toxins purified from clostridial strains, are the only two BoNT serotypes that are currently approved by regulatory agencies for use in humans for applications ranging, among others, from spasticity, bladder dysfunction, or hyperhidrosis (for BoNT/A) (see for example:

https://www.medicines.org.uk/emc/medicine/112, https://www.medicines.org.uk/emc/medicine/870, https://www.medicines.org.uk/emc/medicine/2162, herein incorporated by reference in their entirety) to cervical dystonia (for BoNT/B) (see for example, https://www.medicines.org.uk/emc/medicine/20568, herein incorporated by reference in its entirety).

In contrast to a cytotoxic protease (e.g. ricin, diphtheria toxin, pseudomonas exotoxin), which acts by killing its natural target cell, clostridial neurotoxins are non-cytotoxic proteases acting by transiently incapacitating the cellular function of its natural target cell. Importantly, a non-cytotoxic protease does not kill the natural target cell upon which it acts. In addition to clostridial neurotoxins (e.g. botulinum neurotoxin, marketed under names such as Dysport™, Neurobloc™, and Botox™), some of the best known examples of non-cytotoxic proteases include IgA proteases (see, for example, WO99/032272), and antarease proteases (see, for example, WO2011/022357).

The term “clostridial neurotoxin” as used herein means any polypeptide that enters a neuron and inhibits neurotransmitter release. This process encompasses the binding of the neurotoxin to a low or high affinity receptor, the internalisation of the neurotoxin, the translocation of the endopeptidase portion of the neurotoxin into the cytoplasm and the enzymatic modification of the neurotoxin substrate. More specifically, the term “neurotoxin” encompasses any polypeptide produced by Clostridium bacteria (clostridial neurotoxins) that enters a neuron and inhibits neurotransmitter release, and such polypeptides produced by recombinant technologies or chemical techniques. Preferably, the clostridial neurotoxin is a botulinum neurotoxin (BoNT).

BoNT serotypes A to G can be distinguished based on inactivation by specific neutralising anti-sera, with such classification by serotype correlating with percentage sequence identity at the amino acid level. BoNT proteins of a given serotype are further divided into different subtypes on the basis of amino acid percentage sequence identity.

An example of a BoNT/A neurotoxin amino acid sequence is provided as SEQ ID NO: 1 (UniProt accession number A5HZZ9) and SEQ ID NO: 13, which are encoded by the nucleotide sequence provided as SEQ ID NO: 12. An example of a BoNT/B neurotoxin amino acid sequence is provided as SEQ ID NO: 2 (UniProt accession number B11 INP5). An example of a BoNT/C neurotoxin amino acid sequence is provided as SEQ ID NO: 3 (UniProt accession number P18640). An example of a BoNT/D neurotoxin amino acid sequence is provided as SEQ ID NO: 4 (UniProt accession number P19321). An example of a BoNT/E neurotoxin amino acid sequence is provided as SEQ ID NO: 5 (accession number WP_003372387). An example of a BoNT/F neurotoxin amino acid sequence is provided as SEQ ID NO: 6 (UniProt accession number Q57236) or as SEQ ID NO: 9 (UniProt/UniParc accession number UP10001 DE3DAC). An example of a BoNT/G neurotoxin amino acid sequence is provided as SEQ ID NO: 7 (accession number WP_039635782). An example of a BoNT/D-C neurotoxin amino acid sequence is provided as SEQ ID NO: 8 (accession number BAM65681). An example of a BoNT/X neurotoxin amino acid sequence is provided as SEQ ID NO: 11 (accession number BAQ12790.1). Preferably BoNT is BoNT/A, more preferably wild-type BoNT/A.

The term “H_{C} domain” as used herein refers to a functionally distinct region of the neurotoxin heavy chain with a molecular weight of approximately 50 kDa that enables the binding of the neurotoxin to a receptor located on the surface of the target cell. The H_{C} domain consists of two structurally distinct subdomains, the “H_{CN} subdomain” (N-terminal part of the H_{C} domain) and the “H_{CC} subdomain” (C-terminal part of the Ho domain, also named H_{CC} domain), each of which having a molecular weight of approximately 25 kDa. A H_{CC} domain is capable of binding to a clostridial neurotoxin protein receptor.

The term “LH_{N} domain” as used herein refers to a neurotoxin region that is distinct from the H_{c} domain, and which consists of an endopeptidase domain (“L” or “light chain”) and of a domain responsible for translocation of the endopeptidase into the cytoplasm (H_{N} domain of the heavy chain). An endopeptidase domain (“L” or “light chain”) is capable of cleaving a SNARE protein.

Exemplary L, H_{N}, H_{CN} and H_{CC} domains are shown in Table 1.

TABLE 1
Exemplary L, H_{N}, H_{CN} and H_{CC} domains

| | Accession | SEQ ID | | | | |
|---|---|---|---|---|---|---|
| BoNT | Number | NO: | L | H_{N} | H_{CN} | H_{CC} |
| BoNT/A1 | A5HZZ9 | 1 | 1-448 | 449-872 | 873-1094 | 1095-1296 |
| BoNT/B1 | B1INP5 | 2 | 1-441 | 442-859 | 860-1081 | 1082-1291 |
| BoNT/C1 | P18640 | 3 | 1-449 | 450-867 | 868-1095 | 1096-1291 |
| BoNT/D | P19321 | 4 | 1-442 | 443-863 | 864-1082 | 1083-1276 |
| BoNT/E1 | WP_003372387 | 5 | 1-423 | 424-846 | 847-1069 | 1070-1252 |
| BoNT/F1 | Q57236 | 6 | 1-439 | 440-865 | 866-1087 | 1088-1278 |
| BoNT/F7 | UPI0001DE3DAC | 9 | 1-508 | 509-862 | 863-1076 | 1077-1268 |
| BoNT/G | WP_039635782 | 7 | 1-446 | 447-864 | 865-1089 | 1090-1297 |
| BoNT/DC | BAM65681 | 8 | 1-442 | 443-863 | 864-1091 | 1092-1285 |

| | | | | | |
|---|---|---|---|---|---|
| BoNT/X | BAQ12790.1 | 11 | 1-439 | 440-892 | 893-1306 |

The above-identified reference sequences should be considered a guide, as slight variations may occur according to sub-serotypes. By way of example, US 2007/0166332 (hereby incorporated by reference in its entirety) cites slightly different clostridial sequences.

The term “activation loop” refers to a polypeptide domain comprising a proteolytic cleavage site. Activation loops of neurotoxins have been described in the art, such as in WO2016156113 (hereby incorporated by reference in its entirety).

In one embodiment, the clostridial neurotoxin consists of or comprises an amino acid sequence having at least 70%, preferably at least 75%, 80%, 85%, 90%, 95%, 99% or 100% sequence identity to any of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, or SEQ ID NO: 11.

In one embodiment, the clostridial neurotoxin consists of or comprises an amino acid sequence having at least 70%, preferably at least 75%, 80%, 85%, 90%, 95%, 99% or 100% sequence identity to SEQ ID NO: 1.

In one embodiment, the clostridial neurotoxin consists of or comprises an amino acid sequence of SEQ ID NO: 1 (e.g. BoNT/A).

In one embodiment, the clostridial neurotoxin is a chimeric neurotoxin.

The term “chimeric neurotoxin” as used herein means a neurotoxin comprising one or more domains originating from a first neurotoxin and one or more domains originating from a second neurotoxin. For example, a chimeric neurotoxin may comprise an LH_{N} domain originating from a first neurotoxin serotype or subtype and a H_{C} domain originating from a second neurotoxin serotype or subtype. Another example of a chimeric neurotoxin is a neurotoxin comprising an LH_{N} H_{CN} domain originating from a first neurotoxin serotype or subtype and a H_{CC} domain originating from a second neurotoxin serotype or subtype. A further example of a chimeric neurotoxin is a neurotoxin comprising an LH_{N} domain from a first neurotoxin serotype or subtype and an activation loop from a second neurotoxin serotype or subtype. Examples of chimeric neurotoxins are provided in WO2017191315 and WO2016156113, both herein incorporated by reference in their entirety.

For example, the chimeric neurotoxin may comprise a LH_{N} domain from a first neurotoxin covalently linked to a Ho domain from a second neurotoxin, preferably wherein said first and second neurotoxins are different, wherein the C-terminal amino acid residue of said LH_{N}domain corresponds to the first amino acid residue of the 3₁₀ helix separating the LH_{N} and Ho domains in said first neurotoxin, and wherein the N-terminal amino acid residue of said H_{c} domain corresponds to the second amino acid residue of the 3₁₀ helix separating the LH_{N} and H_{C} domains in said second neurotoxin.

In one embodiment, the clostridial neurotoxin is a chimeric neurotoxin which comprises an Ho domain from a BoNT/B and an LH_{N} domain from a BoNT/A, BoNT/C, BoNT/D, BoNT/E, BoNT/F, or BoNT/G.

For example, in one embodiment, the Ho domain consists of or comprises an amino acid sequence corresponding to amino acid residues 860 to 1291 of SEQ ID NO: 2 (e.g. BoNT/B), or an amino acid sequence having at least 70%, preferably at least 75%, 80%, 85%, 90%, 95% or 99% sequence identity thereto, and the LH_{N} domain consists of or comprises an amino acid sequence selected from the group consisting of:
amino acid residues 1 to 872 of SEQ ID NO: 1 (e.g. BoNT/A), or a sequence having at least 70%, preferably at least 75%, 80%, 85%, 90%, 95% or 99% sequence identity thereto,
amino acid residues 1 to 867 of SEQ ID NO: 3, or a sequence having at least 70%, preferably at least 75%, 80%, 85%, 90%, 95% or 99% sequence identity thereto,
amino acid residues 1 to 863 of SEQ ID NO: 4, or a sequence having at least 70%, preferably at least 75%, 80%, 85%, 90%, 95% or 99% sequence identity thereto,
amino acid residues 1 to 846 of SEQ ID NO: 5, or a sequence having at least 70%, preferably at least 75%, 80%, 85%, 90%, 95% or 99% sequence identity thereto,
amino acid residues 1 to 865 of SEQ ID NO: 6, or a sequence having at least 70%, preferably at least 75%, 80%, 85%, 90%, 95% or 99% sequence identity thereto,
amino acid residues 1 to 864 of SEQ ID NO: 7, or a sequence having at least 70%, preferably at least 75%, 80%, 85%, 90%, 95% or 99% sequence identity thereto,
amino acid residues 1 to 863 of SEQ ID NO: 8, or a sequence having at least 70%, preferably at least 75%, 80%, 85%, 90%, 95% or 99% sequence identity thereto, and
amino acid residues 1 to 862 of SEQ ID NO: 9, or a sequence having at least 70%, preferably at least 75%, 80%, 85%, 90%, 95% or 99% sequence identity thereto.

In a preferred embodiment, the clostridial neurotoxin is a chimeric neurotoxin which comprises an H_{C} domain from a BoNT/B and an LH_{N} domain from a BoNT/A.

In a more preferred embodiment, the H_{C} domain consists of or comprises an amino acid sequence corresponding to amino acid residues 860 to 1291 of SEQ ID NO: 2, or an amino acid sequence having at least 70%, preferably at least 75%, 80%, 85%, 90%, 95% or 99% sequence identity thereto, and the LH_{N} domain comprises an amino acid sequence corresponding to amino acid residues 1 to 872 of SEQ ID NO: 1, or an amino acid sequence having at least 70%, preferably at least 75%, 80%, 85%, 90%, 95% or 99% sequence identity thereto.

In embodiments where the clostridial neurotoxin comprises an H_{C} domain from a BoNT/B (for example, where the clostridial neurotoxin is BoNT/B, or a chimeric neurotoxin which comprises an H_{C} domain from a BoNT/B), the clostridial neurotoxin may have one or more modifications in the amino acid sequence of the heavy chain (such as in the H_{C} domain) providing a “modified heavy chain”, preferably wherein said modified heavy chain binds to target nerve cells with a higher (or lower) affinity than the native neurotoxin. Such modifications in the H_{C} domain can include modifications of amino acid residues in the ganglioside binding site of the H_{CC} domain that can alter binding to the ganglioside of the target nerve cell, and/or modifications of amino acid residues in the protein receptor binding site of the H_{CC} domain that can alter binding to the protein receptor of the target nerve cell. Examples of such modified neurotoxins are described in WO2006027207 and WO2006114308, both of which are hereby incorporated by reference in their entirety.

A clostridial neurotoxin having one or more modifications in the amino acid sequence of the heavy chain is referred to as a “modified clostridial neurotoxin” herein.

In one embodiment of a modified clostridial neurotoxin according to the invention, the H_{cc} domain from a BoNT/B is modified as compared to the natural H_{CC} domain of said BoNT serotypes.

In a preferred embodiment, the H_{CC} domain from a BoNT/B neurotoxin comprises at least one amino acid residue mutation which increases the binding affinity of said H_{CC} domain for human Syt II as compared to the natural BoNT/B H_{CC} domain. Still, preferably, said at least one amino acid residue mutation increases the binding affinity of said H_{CC} domain for human Syt II by at least 50% as compared to the natural BoNT/B H_{CC} domain.

Such suitable amino acid residue mutations in the BoNT/B H_{CC} domain have been described in the art in WO2013180799 and WO2016154534, both herein incorporated by reference in their entirety.

In particular, said at least one amino acid residue mutation suitable for increasing the binding affinity of the BoNT/B H_{CC} domain for human Syt II by at least 50% as compared to the natural BoNT/B H_{CC} domain is an amino acid residue substitution, addition or deletion selected from the group consisting of: 1118M, 1183M, 1191M, 11911, 1191Q, 1191T, 1199Y, 1199F, 1199L, 1201V, 1191C, 1191V, 1191L, 1191Y, 1199W, 1199E, 1199H, 1178Y, 1178Q, 1178A, 1178S, 1183C, 1183P and any combinations thereof. Preferably, said at least one amino acid residue mutation in the BoNT/B H_{CC} domain consists of two amino acid residue substitutions, additions or deletions selected from the group consisting of: 1191M and 1199L, 1191M and 1199Y, 1191M and 1199F, 1191Q and 1199L, 1191Q and 1199Y, 1191Q and 1199F, 1191M and 1199W, 1191M and 1178Q, 1191C and 1199W, 1191C and 1199Y, 1191C and 1178Q, 1191Q and 1199W, 1191V and 1199W, 1191V and 1199Y, or 1191 V and 1178Q. Still preferably, said at least one amino acid residue mutation in the BoNT/B H_{CC} domain consists of the three amino acid residue substitutions, additions or deletions: 1191M, 1199W and 1178Q. More preferably, said at least one amino acid residue mutation in BoNT/B H_{CC} domain consists of the two amino acid residue substitutions, additions or deletions: 1191M and 1199Y.

In a more preferred embodiment, said at least one amino acid residue mutation suitable for increasing the binding affinity of the BoNT/B H_{CC} domain for human Syt II by at least 50% as compared to the natural BoNT/B H_{CC} domain is an amino acid residue substitution selected from the group consisting of: V1118M, Y1183M, E1191M, E11911, E1191Q, E1191T, S1199Y, S1199F, S1199L, S1201V, E1191C, E1191V, E1191L, E1191Y, S1199W, S1199E, S1199H, W1178Y, W1178Q, W1178A, W1178S, Y1183C, Y1183P and any combinations thereof. Preferably, said at least one amino acid residue mutation in the BoNT/B H_{CC} domain consists of two amino acid residue substitutions selected from the group consisting of: E1191M and S1199L, E1191M and S1199Y, E1191M and S1199F, E1191Q and S1199L, E1191Q and S1199Y, E1191Q and S1199F, E1191M and S1199W, E1191M and W1178Q, E1191C and S1199W, E1191C and S1199Y, E1191C and W1178Q, E1191Q and S1199W, E1191V and S1199W, E1191V and S1199Y, or E1191V and W1178Q. Still preferably, said at least one amino acid residue mutation in the BoNT/B H_{CC} domain consists of the three amino acid residue substitutions:E1191M, S1199W and W1178Q. More preferably, said at least one amino acid residue mutation in BoNT/B H_{CC} domain consists of the two amino acid residue substitutions:E1191M and S1199Y.

In a preferred embodiment, the BoNT/B H_{CC} domain to be modified corresponds to amino acid residues 1082 to 1291 of SEQ ID NO: 2 (natural BoNT/B H_{CC} domain), or to an amino acid sequence having at least 70%, preferably at least 80%, 85%, 90%, 95% or 99% sequence identity thereto.

In one embodiment, the clostridial neurotoxin of the present invention can be both chimeric and modified, as described above. For example, in a preferred embodiment, the clostridial neurotoxin comprises (or consists of) the amino acid sequence SEQ ID NO: 10, or an amino acid sequence having at least 70%, preferably at least 75%, 80%, 85%, 90%, 95% or 99% sequence identity thereto.

In one embodiment, the clostridial neurotoxin of the present invention can be both chimeric and modified, as described above. For example, in a preferred embodiment, the clostridial neurotoxin comprises (or consists of) the amino acid sequence SEQ ID NO: 10 (e.g. BoNT/AB_{MY}), or an amino acid sequence having at least 70%, preferably at least 75%, 80%, 85%, 90%, 95% or 99% sequence identity thereto.

The clostridial neurotoxin of the present invention can be produced using recombinant technologies. Thus, in one embodiment, the clostridial neurotoxin of the invention is a recombinant clostridial neurotoxin.

Employing such recombinant neurotoxins may advantageously widen the choice of clostridial neurotoxin to employ in the methods described herein, for example chosen based properties such potency and duration of action as deemed appropriate for any given surgery. Suitable (known) recombinant clostridial neurotoxins include modified botulinum neurotoxin A (BoNT/A) which preferably has a longer duration of action when compared to unmodified BoNT/A (e.g. Dysport®). Said duration of action may be at least 1.25×, 1.5×, 1.75×, 2.0×, or 2.25× greater. The duration of action of modified BoNT/A may be between 6 and 9 months. For example, a duration of action may be at least: 4.5 months (from onset), 5.0 months, 5.5 months, 6 months, 6.5 months, 7.0 months, 7.5 months, 8.0 months, 8.5 months or 9.0 months.

Suitable modified BoNT/A polypeptides (and nucleotide sequences encoding the same, where present) are described in WO 2015/004461 A1 and WO 2017/191315, both of which are incorporated herein by reference in their entirety.

In more detail, in one embodiment, the clostridial neurotoxin is a modified recombinant BoNT/A neurotoxin. In one embodiment, the modified BoNT/A comprises a modification at one or more amino acid residue(s) selected from: ASN 886, ASN 905, GLN 915, ASN 918, GLU 920, ASN 930, ASN 954, SER 955, GLN 991, GLU 992, GLN 995, ASN 1006, ASN 1025, ASN 1026, ASN 1032, ASN 1043, ASN 1046, ASN 1052, ASP 1058, HIS 1064, ASN 1080, GLU 1081, GLU 1083, ASP 1086, ASN 1188, ASP 1213, GLY 1215, ASN 1216, GLN 1229, ASN 1242, ASN 1243, SER 1274, and THR 1277, preferably wherein the modification is selected from: (i) substitution of an acidic surface exposed amino acid residue with a basic amino acid residue; (ii) substitution of an acidic surface exposed amino acid residue with an uncharged amino acid residue; (iii) substitution of an uncharged surface exposed amino acid residue with a basic amino acid residue; (iv) insertion of a basic amino acid residue; and (v) deletion of an acidic surface exposed amino acid residue.

The modification may be a modification when compared to unmodified BoNT/A shown as SEQ ID NO: 1, wherein the amino acid residue numbering is determined by alignment with SEQ ID NO: 1. As the presence of a methionine residue at position 1 of SEQ ID NO: 1 (as well as the SEQ ID NOs corresponding to modified BoNT/A polypeptides described herein) is optional, the skilled person will take the presence/absence of the methionine residue into account when determining amino acid residue numbering. For example, where SEQ ID NO: 1 includes a methionine, the position numbering will be as defined above (e.g. ASN 886 will be ASN 886 of SEQ ID NO: 1). Alternatively, where the methionine is absent from SEQ ID NO: 1 the amino acid residue numbering should be modified by −1 (e.g. ASN 886 will be ASN 885 of SEQ ID NO: 1). Similar considerations apply when the methionine at position 1 of the other polypeptide sequences described herein is present/absent, and the skilled person will readily determine the correct amino acid residue numbering using techniques routine in the art. The same applies to any other BoNT described herein (e.g. the chimeric BoNTs described above).

The amino acid residue(s) indicated for modification are surface exposed amino acid residue(s).

The modified BoNT/A may be encoded by a nucleic acid sequence having at least 70% sequence identity to a nucleic acid sequence selected from SEQ ID NOs: 14, 16, 18, and 20. For example, a nucleic acid sequence having at least 80%, 90%, 95% or 99.9% sequence identity to a nucleic acid sequence selected from SEQ ID NOs: 14, 16, 18, and 20. Preferably, a modified BoNT/A for use in the invention may be encoded by a nucleic acid sequence comprising (or consisting of) SEQ ID NOs: 14, 16, 18 or 20. The modified BoNT/A may comprise a polypeptide sequence having at least 70% sequence identity to a polypeptide sequence selected from SEQ ID NOs: 15, 17, 19, and 21. For example, a polypeptide sequence having at least 80%, 90%, 95% or 99.9% sequence identity to a polypeptide sequence selected from SEQ ID NOs: 15, 17, 19, and 21. Preferably, a modified BoNT/A for use in the invention may comprise (more preferably consist of) a polypeptide sequence selected from SEQ ID NOs: 15, 17, 19, and 21.

The term “one or more amino acid residue(s)” when used in the context of modified BoNT/A preferably means at least 2, 3, 4, 5, 6 or 7 of the indicated amino acid residue(s). Thus, a modified BoNT/A may comprise at least 2, 3, 4, 5, 6 or 7 (preferably 7) modifications at the indicated amino acid residue(s). A modified BoNT/A may comprise 1-30, 3-20, or 5-10 amino acid modifications. More preferably, the term “one or more amino acid residue(s)” when used in the context of modified BoNT/A means all of the indicated amino acid residue(s).

Preferably, beyond the one or more amino acid modification(s) at the indicated amino acid residue(s), the modified BoNT/A does not contain any further amino acid modifications when compared to SEQ ID NO: 1.

Most preferably, a modified BoNT/A comprises (more preferably consists of) a modification at one or more amino acid residue(s) selected from: ASN 886, ASN 930, SER 955, GLN 991, ASN 1026, ASN 1052, and GLN 1229. The modified BoNT/A may be encoded by a nucleic acid sequence having at least 70% sequence identity to SEQ ID NO: 14. For example, a nucleic acid sequence having at least 80%, 90%, 95% or 99.9% sequence identity to SEQ ID NO: 14. Preferably, a modified BoNT/A for use in the invention may be encoded by a nucleic acid comprising (or consisting of) SEQ ID NO: 14. The modified BoNT/A may comprise a polypeptide sequence having at least 70% sequence identity to SEQ ID NO: 15. For example, a polypeptide sequence having at least 80%, 90%, 95% or 99.9% sequence identity to SEQ ID NO: 15. Preferably, a modified BoNT/A for use in the invention may comprise (more preferably consist of) SEQ ID NO: 15.

The modification may be selected from: (i) substitution of an acidic surface exposed amino acid residue with a basic amino acid residue; (ii) substitution of an acidic surface exposed amino acid residue with an uncharged amino acid residue; (iii) substitution of an uncharged surface exposed amino acid residue with a basic amino acid residue; (iv) insertion of a basic amino acid residue; and (v) deletion of an acidic surface exposed amino acid residue.

A modification as indicated above results in a modified BoNT/A that has an increased positive surface charge and increased isoelectric point when compared to the corresponding unmodified BoNT/A.

The isoelectric point (pl) is a specific property of a given protein. In more detail, the isoelectric point (pl) is defined as the pH value at which a protein displays a net charge of zero. An increase in pl means that a higher pH value is required for the protein to display a net charge of zero. Thus, an increase in pl represents an increase in the net positive charge of a protein at a given pH. Conversely, a decrease in pl means that a lower pH value is required for the protein to display a net charge of zero. Thus, a decrease in pl represents a decrease in the net positive charge of a protein at a given pH.

Methods of determining the pl of a protein are known in the art and would be familiar to a skilled person. By way of example, the pl of a protein can be calculated from the average pKa values of each amino acid present in the protein (“calculated pl”). Such calculations can be performed using computer programs known in the art, such as the Compute pl/MW Tool from ExPASy (https://web.expasy.org/compute_pi/), which is the preferred method for calculating pl in accordance with the present invention. Comparisons of pl values between different molecules should be made using the same calculation technique/program. Where appropriate, the calculated pl of a protein can be confirmed experimentally using the technique of isoelectric focusing (“observed pl”). This technique uses electrophoresis to separate proteins according to their pl. Isoelectric focusing is typically performed using a gel that has an immobilised pH gradient. When an electric field is applied, the protein migrates through the pH gradient until it reaches the pH at which it has zero net charge, this point being the pl of the protein. Results provided by isoelectric focusing are typically relatively low-resolution in nature, and thus the present inventors believe that results provided by calculated pl (as described above) are more appropriate to use.

Throughout the present specification, “pl” means “calculated pl” unless otherwise stated. The pl of a protein may be increased or decreased by altering the number of basic and/or acidic groups displayed on its surface. This can be achieved by modifying one or more amino acids of the protein. For example, an increase in pl may be provided by reducing the number of acidic residues, or by increasing the number of basic residues.

A modified BoNT/A of the invention may have a pl value that is at least 0.2, 0.4, 0.5 or 1 pl units higher than that of an unmodified BoNT/A (e.g. SEQ ID NO: 1). Preferably, a modified BoNT/A may have a pl of at least 6.6, e.g. at least 6.8.

The properties of the 20 standard amino acids are indicated in the table below:

| Amino Acid | | | Side Chain | Amino Acid | | | Side Chain |
|---|---|---|---|---|---|---|---|
| Aspartic acid | Asp | D | Charged | Methionine | Met | M | Uncharged |
| | | | (acidic) | | | | (polar) |
| Glutamic | Glu | E | Charged | Tryptophan | Trp | W | Uncharged |
| acid | | | (acidic) | | | | (polar) |
| Arginine | Arg | R | Charged | Cysteine | Cys | C | Uncharged |
| | | | (basic) | | | | (polar) |
| Lysine | Lys | K | Charged | Alanine | Ala | A | Uncharged |
| | | | (basic) | | | | (hydrophobic) |
| Histidine | His | H | Uncharged | Glycine | Gly | G | Uncharged |
| | | | (polar) | | | | (hydrophobic) |
| Asparagine | Asn | N | Uncharged | Valine | Val | V | Uncharged |
| | | | (polar) | | | | (hydrophobic) |
| Glutamine | Gln | Q | Uncharged | Leucine | Leu | L | Uncharged |
| | | | (polar) | | | | (hydrophobic) |
| Serine | Ser | S | Uncharged | Isoleucine | Ile | I | Uncharged |
| | | | (polar) | | | | (hydrophobic) |
| Threonine | Thr | T | Uncharged | Proline | Pro | P | Uncharged |
| | | | (polar) | | | | (hydrophobic) |
| Tyrosine | Tyr | Y | Uncharged | Phenylalanine | Phe | F | Uncharged |
| | | | (polar) | | | | (hydrophobic) |

The following amino acids are considered charged amino acids: aspartic acid (negative), glutamic acid (negative), arginine (positive), and lysine (positive).

At a pH of 7.4, the side chains of aspartic acid (pKa 3.1) and glutamic acid (pKa 4.1) have a negative charge, while the side chains of arginine (pKa 12.5) and lysine (pKa 10.8) have a positive charge. Aspartic acid and glutamic acid are referred to as acidic amino acid residues. Arginine and lysine are referred to as basic amino acid residues.

The following amino acids are considered uncharged, polar (meaning they can participate in hydrogen bonding) amino acids: asparagine, glutamine, histidine, serine, threonine, tyrosine, cysteine, methionine, and tryptophan. The following amino acids are considered uncharged, hydrophobic amino acids: alanine, valine, leucine, isoleucine, phenylalanine, proline, and glycine.

In an amino acid insertion, an additional amino acid residue (one that is not normally present) is incorporated into the BoNT/A polypeptide sequence, thus increasing the total number of amino acid residues in said sequence. In an amino acid deletion, an amino acid residue is removed from the clostridial toxin amino acid sequence, thus reducing the total number of amino acid residues in said sequence.

Preferably, the modification is a substitution, which advantageously maintains the same number of amino acid residues in the modified BoNT/A. In an amino acid substitution, an amino acid residue that forms part of the BoNT/A polypeptide sequence is replaced with a different amino acid residue. The replacement amino acid residue may be one of the 20 standard amino acids, as described above. Alternatively, the replacement amino acid in an amino acid substitution may be a non-standard amino acid (an amino acid that is not part of the standard set of 20 described above). By way of example, the replacement amino acid may be a basic non-standard amino acid, e.g. L-Ornithine, L-2-amino-3-guanidinopropionic acid, or D-isomers of Lysine, Arginine and Ornithine). Methods for introducing non-standard amino acids into proteins are known in the art and include recombinant protein synthesis using E. coli auxotrophic expression hosts.

In one embodiment, the substitution is selected from: substitution of an acidic amino acid residue with a basic amino acid residue, substitution of an acidic amino acid residue with an uncharged amino acid residue, and substitution of an uncharged amino acid residue with a basic amino acid residue. In one embodiment, wherein the substitution is a substitution of an acidic amino acid residue with an uncharged amino acid residue, the acidic amino acid residue is replaced with its corresponding uncharged amide amino acid residue (i.e. aspartic acid is replaced with asparagine, and glutamic acid is replaced with glutamine).

Preferably, the basic amino acid residue is a lysine residue or an arginine residue. In other words, the substitution is substitution with lysine or arginine. Most preferably, the modification is substitution with lysine.

Following modification in accordance with the invention, the modified BoNT/A is capable of binding to the target cell receptors that unmodified BoNT/A (e.g. SEQ ID NO: 1) binds.

Having just described suitable modified (recombinant) BoNT/A neurotoxins e.g. have long duration of action, described below are suitable modified (recombinant) BoNT/E neurotoxins, which may be comparatively faster acting and/or having a shorter term of action. This again demonstrates the advantageous flexibility provided by the clostridial neurotoxin based therapy of the invention. For example, for less invasive surgeries (e.g. where post-operative pain would not be expected to present for long), such BoNT/E may be employed to provide a shorter duration of action.

The BoNT/E (e.g. rBoNT/E) may comprise a polypeptide sequence having at least 70% (preferably at least 80%; more preferably at least 90%) sequence identity to SEQ ID NO: 5, with the proviso that the polypeptide sequence includes one or more (for example, one or more, two or more, three or more, four or more, five or more, six or more, seven or more, or eight; preferably all eight) of the following amino acids (wherein the amino acid position numbering starts with the N-terminal methionine amino acid residue and ends with the C-terminal amino acid residue of the BoNT/E protein): glycine at position 177; serine at position 198; alanine at position 340; leucine at position 773; leucine at position 963; glutamine at position 964; alanine at position 967; asparagine at position 1195.

Said amino acids may be substitutions (e.g. mutations) relative to a wild-type BoNT/E polypeptide sequence (such as the sequence of UniProt 000496). For example: the glycine at position 177 may be an arginine to glycine substitution (R177G); the serine at position 198 may be a C198S substitution; the alanine at position 340 may be a R340A substitution; the leucine at position 773 may be a 1173L substitution; the leucine at position 963 may be a F963L substitution; the glutamine at position 964 may be a E964Q substitution; the alanine at position 967 may be a R967A substitution; and/or the asparagine at position 1195 may be an insertion (e.g. an insertion between G1194 and N1195 of a wild-type BoNT/E sequence, such as the polypeptide sequence of UniProt 000496).

In one embodiment, the presence of said one or more amino acids, as described above, provides a BoNT/E protein having improved solubility as compared to a BoNT/E protein lacking said amino acid(s). Said improved solubility increases the yield of the protein in a heterologous expression system, such as an E. coliexpression system.

In one embodiment, the presence of said one or more amino acids, as described above, provides a BoNT/E protein having improved potency as compared to a BoNT/E protein lacking said amino acid(s). Said improved potency may preferably be improved in vivo, potency (more preferably improved in vivo potency in a human subject).

In one embodiment BoNT/E is one described in (or encoded by a nucleotide sequence described in) WO 2014/068317 A1, which is incorporated herein by reference.

Preferably, the clostridial neurotoxin (e.g. for use as described herein) is part of a pharmaceutical composition together with at least one pharmaceutically acceptable carrier. By “pharmaceutically acceptable carrier”, it is meant herein any component that is compatible with the other ingredients of the pharmaceutical composition, in particular with the clostridial neurotoxin, and which is not deleterious to the human patient. The pharmaceutically acceptable carrier can be selected on the basis of the desired route of administration, in accordance with standard pharmaceutical practices, and include, without limitation, excipients, diluents, adjuvants, propellants and salts.

Accordingly, the present invention further relates to a pharmaceutical composition for use in the treatment of post-operative surgical pain and/or anxiety in a human patient, wherein said composition comprises the clostridial neurotoxin of the invention and at least one pharmaceutically acceptable carrier, and the dose of the clostridial neurotoxin to be administered to the patient is as described above. Also encompassed are corresponding uses and methods of treating post-operative surgical pain and/or anxiety comprising administering a pharmaceutical composition of the invention to a human patient. In another embodiment, the present invention relates to a pharmaceutical composition for use in promoting post-operative wellness, wherein the post-operative wellness is reduced post-operative surgical pain and anxiety.

The clostridial neurotoxin of the present invention may preferably be formulated for intradermal administration.

A preferred route of administration is via intradermal administration. Preferably, intradermal administration means intradermal injection.

Preferably, said BoNT treating the post-operative surgical pain and/or post-operative anxiety is a purified BoNT. As used herein, the term “purified BoNT” means a botulinum neurotoxin purified from a clostridial strain which naturally produces it (naturally-occurring clostridial strain) or purified using recombinant technology. The purified BoNT/A may be associated with complexing proteins or free of complexing proteins, but is preferably free of complexing proteins. Thus, in one embodiment, the clostridial neurotoxin is associated with BoNT complexing proteins, also known as non-toxic neurotoxin-associated proteins (NAP). In other words, the clostridial neurotoxin is administered to the human patient in association with, or combined with, BoNT complexing proteins. Hence, in one embodiment the clostridial neurotoxin is complexed with one or more BoNT complexing proteins. Examples of commercially available purified and complexing protein-associated BoNT/A include Botox®, Dysport® (associated with BoNT complexing proteins) and Xeomin® (purified).

In another embodiment, the clostridial neurotoxin is free of (or not associated with, or combined with) BoNT complexing proteins. In other words, the clostridial neurotoxin is administered to the human patient without being associated with, or combined with, BoNT complexing proteins.

The doses of clostridial neurotoxin may be measured in nanograms.

Doses of clostridial neurotoxin according to the invention are to be understood as doses of active di-chain clostridial neurotoxin, i.e. without including the quantity of complexing proteins to which the neurotoxin may be associated with. In other words, it refers to the doses of active di-chain clostridial neurotoxin, whether said neurotoxin is administered to the patient in association to, or without, complexing proteins. As well-known to the skilled practitioner, an active di-chain clostridial neurotoxin is capable of binding to a membrane (e.g. cell membrane) receptor, translocating the light chain into the cytoplasm and of cleaving a SNARE protein, while complexing proteins do not display such biological activity (i.e. are not “active”).

Additionally or alternatively, the doses of clostridial neurotoxin may be measured in “Units” (U) of clostridial neurotoxin. For example, the measurement of doses in Units may be particularly suitable when administering BoNT/A (or more particularly, for example, Dysport®).

Indeed, as well known to the skilled practitioner, the potency of a clostridial neurotoxin is related to the quantity (e.g. nanograms) of neurotoxin required to achieve an LD50 (lethal dose 50) unit; one LD50 unit being defined as the median lethal intraperitoneal dose (as measured in mice). However, BoNT pharmaceutical preparations currently on the market contain different amount of 150 kD neurotoxin, but also of LD50 Units. Besides, in these preparations, the neurotoxin may, or may not, be associated with (i.e. combined with) non-toxic neurotoxin-associated proteins (NAP), also known as complexing proteins. For ease of conversion (as reported in Field et. al, “AbobotulinumtoxinA (Dysport®), OnabotulinumtoxinA (Botox®), and IncobotulinumtoxinA (Xeomin®) Neurotoxin Content and Potential Implications for Duration of Response in Patients”. Toxins 2018, 10(12), 535):
100 Units of Botox® (also known as OnabotulinumtoxinA) contains about 0.9 ng of 150 kD BoNT/A, as well as complexing proteins;
500 Units of Dysport® (also known as AbobotulinumtoxinA) contains about 2.69 ng of 150 kD BoNT/A, as well as complexing proteins; 1 Unit of Dysport® contains about 5.38 pg BoNT/A;
100 Units of Xeomin® (also known as IncobotulinumtoxinA) contains about 0.40 ng of 150 kD BoNT/A, with no complexing proteins.

It should be noted that conversion values may vary slightly. For example, conversion values reported in Frevert, 2012 (“Content of botulinum neurotoxin in Botox®/Vistabel®, Dysport®/Azzalure®, and Xeomin®/Bocouture®”; Drugs R D. 2010; 10(2):67-73) are as follows:
100 Units of Botox® (also known as OnabotulinumtoxinA) contains about 0.73 ng of 150 kD BoNT/A, as well as complexing proteins;
100 Units of Dysport® (also known as AbobotulinumtoxinA) contains about 0.65 ng of 150 kD BoNT/A, as well as complexing proteins;
100 Units of Xeomin® (also known as IncobotulinumtoxinA) contains about 0.44 ng of 150 kD BoNT/A, with no complexing proteins;
100 Units of Neurobloc/Myobloc® (also known as RimabotulinumtoxinB) contains about 0.2 ng to about 1 ng of 150 kD BoNT/B, as well as complexing proteins.

The quantity of clostridial neurotoxin can be measured by the skilled practitioner according to methods conventionally used in the art to quantify proteins preferably at nanograms levels, including, among others, mass spectroscopy such as isotopic dilution mass spectroscopy (Muñoz et al., Quantification of protein calibrants by amino acid analysis using isotope dilution mass spectrometry, Anal. Biochem. 2011, 408, 124-131), or fluorimetric assay (Poras et al., Detection and Quantification of Botulinum Neurotoxin Type A by a Novel Rapid In Vitro Fluorimetric Assay, Appl Environ Microbiol. 2009 July; 75(13): 4382-4390).

Intradermal administration may comprise intradermal injection with a needle, such as a 30 gauge needle, preferably wherein the needle (such as a 30 gauge needle) is inserted into dermis of the skin at an angle of about 5°-15° relative to the surface of the skin at the site of surgical intervention (which may be on the flank). The depth of the injection (depth relative to the surface of the skin) may be around 0.2-0.3 (preferably around 0.25) inches.

The clostridial neurotoxin may be administered at the site on the body that is to be subjected to surgical intervention (e.g. a surgical incision, or proximal to the site of surgical incision).

In one embodiment, the clostridial neurotoxin may be administered (such as via intradermal injection or intrathecal injection) at the site of surgical intervention on the patient (e.g. at one or more administration sites at the site of surgical intervention on the patient).

The clostridial neurotoxin may be administered at one or more sites, for example at one or more sites proximal to the site of surgical intervention. A site that is “proximal to the site of surgical intervention” may be situated up to 15 cm from the site of surgical intervention; for example, up to 10 cm from the site of surgical intervention; preferably up to 5 cm from the site of surgical intervention; more preferably up to 1 cm from the site of surgical intervention.

In one embodiment following administration, the clostridial neurotoxin travels by retrograde transport to the spinal cord and effects SNARE protein cleavage (SNAP-25 protein cleavage) in said spinal cord.

In one embodiment when a clostridial neurotoxin is administered at an intradermal site, minimal or no SNARE protein cleavage (SNAP-25 protein cleavage) by said clostridial neurotoxin is observed at or proximal to said intradermal site following administration of the clostridial neurotoxin. In one embodiment observations were made 5-7 days following administration of the clostridial neurotoxin and minimal or no SNARE protein cleavage (SNAP-25 protein cleavage) by said clostridial neurotoxin is observed at or proximal to said intradermal site following administration of the clostridial neurotoxin.

In one embodiment when a clostridial neurotoxin is administered at an intrathecal site, minimal or no SNARE protein cleavage (SNAP-25 protein cleavage) by said clostridial neurotoxin is observed at or proximal to said intrathecal site following administration of the clostridial neurotoxin. In one embodiment observations were made 5-7 days following administration of the clostridial neurotoxin and minimal or no SNARE protein cleavage (SNAP-25 protein cleavage) by said clostridial neurotoxin is observed at or proximal to said intrathecal site following administration of the clostridial neurotoxin.

Thus, a clostridial neurotoxin may be administered distal to a site of surgical intervention to treat post-operative surgical pain and post-operative anxiety.

Thus, in a preferred embodiment, when the post-operative surgical pain is caused by surgical intervention, the clostridial neurotoxin may be administered (such as via intradermal injection or intrathecal injection) at a site distal to the site of surgical intervention (e.g. at one or more administration sites distal to the site of incision on the patient).

The clostridial neurotoxin may be administered at one or more sites distal to the site of surgical intervention, for example at a site at least 15 cm from the site of surgical intervention; at least 50 cm from the site of surgical intervention or at least 100 cm from the site of surgical intervention.

The skilled person would understand that the invention is directed to pre-surgery administration, such that the reference to administration “at or proximal to the site of surgical intervention” refers to administration at or proximal to a site that will be (e.g. subsequently) subjected to surgical intervention once surgery commences. The reference to administration “at a site distal to the site of surgical intervention” refers to administration distal to a site that will be (e.g. subsequently) subjected to surgical intervention once surgery commences.

The clostridial neurotoxin may be administered at up to 15 (preferably up to 10) sites (e.g. sites proximal to the site of surgical intervention). Such sites may traverse the periphery of the site of surgical intervention.

In a preferred embodiment, the dose of the clostridial neurotoxin of the invention to be administered for treating surgical pain in a human patient (i.e. therapeutic dose) is ranging from about 0.00025 ng to about 3 ng.

In a preferred embodiment, the therapeutic dose of the clostridial neurotoxin is ranging from about 0.0003 ng to about 2 ng, preferably from about 0.0004 ng to about 1.5 ng, from about 0.0005 ng to about 1 ng, still preferably from about 0.0006 ng to about 0.5 ng of said clostridial neurotoxin.

For example, the dose (e.g. total dose) of the clostridial neurotoxin comprising a BoNT/A is preferably ranging from about 1 ng to about 2 ng.

The patient may be administered 100-500 U of the clostridial neurotoxin. For example, the patient may be administered 150-300 U of the clostridial neurotoxin; preferably 175-250 U; more preferably about 200 U.

The patient may be administered 80-250 picograms (pg) of the clostridial neurotoxin per kilogram (kg) of the patient's bodyweight (e.g. 850-250 pg/kg). For example, the patient may be administered 100-200 pg/kg, 115-175 pg/kg, or 130-150 pg/kg.

As described above, the clostridial neurotoxin may be administered at one or more administration sites, for example at more than one administration site. In one embodiment, the patient is administered 2.5-30 U of the clostridial neurotoxin per administration site; preferably wherein the patient is administered 20 U of the clostridial neurotoxin per administration site. For example, 10 administration sites may receive an administration of 20 U per administration site, providing a total administration of 200 U.

The patient may be administered the clostridial neurotoxin at a total dose of 10-170 pg per administration site. In a preferable embodiment, the patient may be administered the clostridial neurotoxin at a dose of 1-14 pg/kg (bodyweight) per administration site.

In another embodiment the therapeutic dose of the clostridial neurotoxin is preferably ranging from about 0.001 ng to about 2 ng. Yet, for example, the therapeutic dose of the clostridial neurotoxin is preferably ranging from about 0.0003 ng to about 0.05 ng.

It will nevertheless be appreciated that the dose range required depends on the precise nature of the clostridial neurotoxin, the maximum tolerated dose in the particular subject (e.g. human subject), the skin condition, the route of administration, the nature of the formulation, the age of the patient, the weight of the patient, the nature, extent or severity of the patient's condition, contraindications, if any, and the judgement of the attending physician. Variations in these dosage levels can be adjusted using standard empirical routines for optimisation.

In one embodiment, a patient is administered a monotherapy based on a single botulinum neurotoxin serotype (e.g. BoNT/A). Thus in one embodiment, the present invention employs the use of a single botulinum neurotoxin serotype (e.g. BoNT/A).

Embodiments related to the various methods of the invention are intended to be applied equally to other methods, the clostridial neurotoxins, e.g. engineered clostridial neurotoxins (whether in single-chain or di-chain forms), uses or pharmaceutical compositions, and vice versa.

Sequence Homology

Any of a variety of sequence alignment methods can be used to determine percent identity, including, without limitation, global methods, local methods and hybrid methods, such as, e.g., segment approach methods. Protocols to determine percent identity are routine procedures within the scope of one skilled in the art. Global methods align sequences from the beginning to the end of the molecule and determine the best alignment by adding up scores of individual residue pairs and by imposing gap penalties. Non-limiting methods include, e.g., CLUSTAL W, see, e.g., Julie D. Thompson et al., CLUSTAL W: Improving the Sensitivity of Progressive Multiple Sequence Alignment Through Sequence Weighting, Position-Specific Gap Penalties and Weight Matrix Choice, 22(22) Nucleic Acids Research 4673-4680 (1994); and iterative refinement, see, e.g., Osamu Gotoh, Significant Improvement in Accuracy of Multiple Protein. Sequence Alignments by Iterative Refinement as Assessed by Reference to Structural Alignments, 264(4) J. Mol. Biol. 823-838 (1996). Local methods align sequences by identifying one or more conserved motifs shared by all of the input sequences. Non-limiting methods include, e.g., Match-box, see, e.g., Eric Depiereux and Ernest Feytmans, Match-Box: A Fundamentally New Algorithm for the Simultaneous Alignment of Several Protein Sequences, 8(5) CABIOS 501-509 (1992); Gibbs sampling, see, e.g., C. E. Lawrence et al., Detecting Subtle Sequence Signals: A Gibbs Sampling Strategy for Multiple Alignment, 262(5131) Science 208-214 (1993); Align-M, see, e.g., Ivo Van Walle et al., Align-M—A New Algorithm for Multiple Alignment of Highly Divergent Sequences, 20(9) Bioinformatics: 1428-1435 (2004).

Thus, percent sequence identity is determined by conventional methods. See, for example, Altschul et al., Bull. Math. Bio. 48: 603-16, 1986 and Henikoff and Henikoff, Proc. Natl. Acad. Sci. USA 89:10915-19, 1992. Briefly, two amino acid sequences are aligned to optimize the alignment scores using a gap opening penalty of 10, a gap extension penalty of 1, and the “blosum 62” scoring matrix of Henikoff and Henikoff (ibid.) as shown below (amino acids are indicated by the standard one-letter codes).

The “percent sequence identity” between two or more nucleic acid or amino acid sequences is a function of the number of identical positions shared by the sequences. Thus, % identity may be calculated as the number of identical nucleotides/amino acids divided by the total number of nucleotides/amino acids, multiplied by 100. Calculations of % sequence identity may also take into account the number of gaps, and the length of each gap that needs to be introduced to optimize alignment of two or more sequences. Sequence comparisons and the determination of percent identity between two or more sequences can be carried out using specific mathematical algorithms, such as BLAST, which will be familiar to a skilled person.

ALIGNMENT SCORES FOR DETERMINING SEQUENCE IDENTITY

A R N D C Q E G H I L K M F P S T W Y V
A 4
R -1 5
N -2 0 6
D -2 -2 1 6
C 0 -3 -3 -3 9
Q -1 1 0 0 -3 5
E -1 0 0 2 -4 2 5
G 0 -2 0 -1 -3 -2 -2 6
H -2 0 1 -1 -3 0 0 -2 8
I -1 -3 -3 -3 -1 -3 -3 -4 -3 4
L -1 -2 -3 -4 -1 -2 -3 -4 -3 2 4
K -1 2 0 -1 -3 1 1 -2 -1 -3 -2 5
M -1 -1 -2 -3 -1 0 -2 -3 -2 1 2-1 5
F -2 -3 -3 -3 -2 -3 -3 -3 -1 0 0 -3 0 6
P -1 -2 -2 -1 -3 -1 -1 -2 -2 -3 -3 -1 -2 -4 7
S 1 -1 1 0 -1 0 0 0 -1 -2 -2 0 -1 -2 -1 4
T 0 -1 0 -1 -1 -1 -1 -2 -2 -1 -1 -1 -1 -2 -1 1 5
W -3 -3 -4 -4 -2 -2 -3 -2 -2 -3 -2 -3 -1 1 -4 -3 -2 1 1
Y -2 -2 -2 -3 -2 -1 -2 -3 2 -1 -1 -2 -1 3 -3 -2 -2 2 7
V 0 -3 -3 -3 -1 -2 -2 -3 -3 3 1 -2 1 -1 -2 -2 0 -3 -1 4

The percent identity is then calculated as:

$$\frac{Total number of identical matches}{\begin{matrix}[ length of the longer sequence plus the \\ number of gaps introduced into the longer \\ sequence in order to align the two sequences ]\end{matrix}} \times 100$$

Substantially homologous polypeptides are characterized as having one or more amino acid substitutions, deletions or additions. These changes are preferably of a minor nature, that is conservative amino acid substitutions (see below) and other substitutions that do not significantly affect the folding or activity of the polypeptide; small deletions, typically of one to about 30 amino acids; and small amino- or carboxyl-terminal extensions, such as an amino-terminal methionine residue, a small linker peptide of up to about 20-25 residues, or an affinity tag.

Conservative Amino Acid Substitutions

Basic: arginine; lysine; histidine

Acidic: glutamic acid; aspartic acid

Polar: glutamine; asparagine

Hydrophobic: leucine; isoleucine; valine

Aromatic: phenylalanine; tryptophan; tyrosine

Small: glycine; alanine; serine; threonine; methionine

In addition to the 20 standard amino acids, non-standard amino acids (such as 4-hydroxyproline, 6-N-methyl lysine, 2-aminoisobutyric acid, isovaline and a-methyl serine) may be substituted for amino acid residues of the polypeptides of the present invention. A limited number of non-conservative amino acids, amino acids that are not encoded by the genetic code, and unnatural amino acids may be substituted for polypeptide amino acid residues. The polypeptides of the present invention can also comprise non-naturally occurring amino acid residues.

Non-naturally occurring amino acids include, without limitation, trans-3-methylproline, 2,4-methano-proline, cis-4-hydroxyproline, trans-4-hydroxy-proline, N-methylglycine, allo-threonine, methyl-threonine, hydroxy-ethylcysteine, hydroxyethylhomo-cysteine, nitro-glutamine, homoglutamine, pipecolic acid, tert-leucine, norvaline, 2-azaphenylalanine, 3-azaphenyl-alanine, 4-azaphenyl-alanine, and 4-fluorophenylalanine. Several methods are known in the art for incorporating non-naturally occurring amino acid residues into proteins. For example, an in vitro system can be employed wherein nonsense mutations are suppressed using chemically aminoacylated suppressor tRNAs. Methods for synthesizing amino acids and aminoacylating tRNA are known in the art. Transcription and translation of plasmids containing nonsense mutations is carried out in a cell free system comprising an E. coli S30 extract and commercially available enzymes and other reagents. Proteins are purified by chromatography. See, for example, Robertson et al., J. Am. Chem. Soc. 113:2722, 1991; Ellman et al., Methods Enzymol. 202:301, 1991; Chung et al., Science 259:806-9, 1993; and Chung et al., Proc. Natl. Acad. Sci. USA 90:10145-9, 1993). In a second method, translation is carried out in Xenopus oocytes by microinjection of mutated mRNA and chemically aminoacylated suppressor tRNAs (Turcatti et al., J. Biol. Chem. 271:19991-8, 1996). Within a third method, E. coli cells are cultured in the absence of a natural amino acid that is to be replaced (e.g., phenylalanine) and in the presence of the desired non-naturally occurring amino acid(s) (e.g., 2-azaphenylalanine, 3-azaphenylalanine, 4-azaphenylalanine, or 4-fluorophenylalanine). The non-naturally occurring amino acid is incorporated into the polypeptide in place of its natural counterpart. See, Koide et al., Biochem. 33:7470-6, 1994. Naturally occurring amino acid residues can be converted to non-naturally occurring species by in vitro chemical modification. Chemical modification can be combined with site-directed mutagenesis to further expand the range of substitutions (Wynn and Richards, Protein Sci. 2:395-403, 1993).

A limited number of non-conservative amino acids, amino acids that are not encoded by the genetic code, non-naturally occurring amino acids, and unnatural amino acids may be substituted for amino acid residues of polypeptides of the present invention.

Essential amino acids in the polypeptides of the present invention can be identified according to procedures known in the art, such as site-directed mutagenesis or alanine-scanning mutagenesis (Cunningham and Wells, Science 244: 1081-5, 1989). Sites of biological interaction can also be determined by physical analysis of structure, as determined by such techniques as nuclear magnetic resonance, crystallography, electron diffraction or photoaffinity labeling, in conjunction with mutation of putative contact site amino acids. See, for example, de Vos et al., Science 255:306-12, 1992; Smith et al., J. Mol. Biol. 224:899-904, 1992; Wlodaver et al., FEBS Lett. 309:59-64, 1992. The identities of essential amino acids can also be inferred from analysis of homologies with related components (e.g. the translocation or protease components) of the polypeptides of the present invention.

Multiple amino acid substitutions can be made and tested using known methods of mutagenesis and screening, such as those disclosed by Reidhaar-Olson and Sauer (Science 241:53-7, 1988) or Bowie and Sauer (Proc. Natl. Acad. Sci. USA 86:2152-6, 1989). Briefly, these authors disclose methods for simultaneously randomizing two or more positions in a polypeptide, selecting for functional polypeptide, and then sequencing the mutagenized polypeptides to determine the spectrum of allowable substitutions at each position. Other methods that can be used include phage display (e.g., Lowman et al., Biochem. 30:10832-7, 1991; Ladner et al., U.S. Pat. No. 5,223,409; Huse, WIPO Publication WO 92/06204) and region-directed mutagenesis (Derbyshire et al., Gene 46:145, 1986; Ner et al., DNA 7:127, 1988).

Unless defined otherwise, all technical and scientific terms used herein have the same meaning as commonly understood by one of ordinary skill in the art to which this disclosure belongs. Singleton, et al., DICTIONARY OF MICROBIOLOGY AND MOLECULAR BIOLOGY, 20 ED., John Wiley and Sons, New York (1994), and Hale & Marham, THE HARPER COLLINS DICTIONARY OF BIOLOGY, Harper Perennial, NY (1991) provide the skilled person with a general dictionary of many of the terms used in this disclosure.

This disclosure is not limited by the exemplary methods and materials disclosed herein, and any methods and materials similar or equivalent to those described herein can be used in the practice or testing of embodiments of this disclosure. Numeric ranges are inclusive of the numbers defining the range. Unless otherwise indicated, any nucleic acid sequences are written left to right in 5′ to 3′ orientation; amino acid sequences are written left to right in amino to carboxy orientation, respectively.

The headings provided herein are not limitations of the various aspects or embodiments of this disclosure.

Amino acids are referred to herein using the name of the amino acid, the three letter abbreviation or the single letter abbreviation. The term “protein”, as used herein, includes proteins, polypeptides, and peptides. As used herein, the term “amino acid sequence” is synonymous with the term “polypeptide” and/or the term “protein”. In some instances, the term “amino acid sequence” is synonymous with the term “peptide”. In some instances, the term “amino acid sequence” is synonymous with the term “enzyme”. The terms “protein” and “polypeptide” are used interchangeably herein. In the present disclosure and claims, the conventional one-letter and three-letter codes for amino acid residues may be used. The 3-letter code for amino acids as defined in conformity with the IUPACIUB Joint Commission on Biochemical Nomenclature (JCBN). It is also understood that a polypeptide may be coded for by more than one nucleotide sequence due to the degeneracy of the genetic code.

Other definitions of terms may appear throughout the specification. Before the exemplary embodiments are described in more detail, it is to be understood that this disclosure is not limited to particular embodiments described, and as such may vary. It is also to be understood that the terminology used herein is for the purpose of describing particular embodiments only, and is not intended to be limiting, since the scope of the present disclosure will be defined only by the appended claims.

Where a range of values is herein provided, it shall be understood that, unless the context clearly dictates otherwise, each intervening value to the tenth of the unit between the upper and lower limits of that range is also specifically disclosed. Each smaller range between any stated value or intervening value in a stated range and any other stated or intervening value in that stated range is encompassed within this disclosure. It shall be further understood that any range of numerical values denoted herein by the expression “from a to b” means the range of numerical values extending from a to b (i.e. including the strict end points a and b).

Besides, the term “about” shall be understood herein as plus or minus (±) 5%, preferably ±4%, ±3%, 2%, 1%, 0.5%, 0.1%, of the numerical value of the number with which it is being used.

It must be noted that as used herein and in the appended claims, the singular forms “a”, “an”, and “the” include plural referents unless the context clearly dictates otherwise. Thus, for example, reference to “a botulinum neurotoxin” includes a plurality of such candidate agents and reference to “the botulinum neurotoxin” includes reference to one or more clostridial neurotoxins and equivalents thereof known to those skilled in the art, and so forth.

The publications discussed herein are provided solely for their disclosure prior to the filing date of the present application. Nothing herein is to be construed as an admission that such publications constitute prior art to the claims appended hereto.

### BRIEF DESCRIPTION OF THE DRAWINGS

Embodiments of the invention will now be described, by way of example only, with reference to the following Figures and Examples.

FIG. 1 shows a schematic of injection sites along the surgical incision, in which Dysport, saline or the reference compound Exparel were injected.

FIG. 2 shows a schematic of the arena used for the open field test for locomotion activity.

FIG. 3 shows the effects from peri-operative administration of intradermal injections of either saline, Exparel (control agent) or different concentrations of Dysport (100, 200 and 400U) on mitigating post-operative surgical pain and anxiety. A Von Frey assay as a measure of surgical pain perception was conducted and results are shown in (A). The horizontal line cutting across the graph, set at 26 g, indicates the baseline for not sensing surgical pain (e.g. a threshold, above which the subject's post-operative surgical pain may be considered treated). Pain can be defined as moderate/severe when the mechanical sensitivity is 0-15 g, mild/moderate when between 15-26 g and little/no pain is sensed when above 26 g. The time taken (in seconds) for pigs to approach their handlers following peri-operative, intradermal administration of Exparel or Dysport is shown in (B). The distress behaviour was scored following peri-operative administration of Exparel or Dysport and is shown in (C).

FIG. 4 shows the mean group total walking distance (in metres) that the animals walked in a period of 5 minutes (A) and the mean percentage of time spent in the central zone of the open field apparatus (B) following the peri-operative intradermal injection of either saline, Exparel (control agent) or different concentrations of Dysport (100, 200 and 400U).

FIG. 5 shows a Von Frey assay of saline (control) versus BoNT/A (Dysport) intradermal administration at 15 days (A), 5 days (B) or 1 day (C) prior to surgery.

FIG. 6 shows the latency (in seconds) of pigs to approach their handler following the intradermal administration of either saline or Dysport (200U/pig) at 15 days (A), 5 days (B) or 1 day (C) prior to surgery. For each timepoint (day) in the bar charts (A-C), the bar on the left (lighter) shows results for Dysport treatment, and the bar on the right (darker) shows results for saline treatment.

FIG. 7 shows the distress behaviour score of pigs following the intradermal administration of either saline or Dysport at 15 days (A), 5 days (B) or 1 day (C) prior to surgery. For each timepoint (day) in the bar charts (A-C), the bar on the left (lighter) shows results for Dysport treatment, and the bar on the right (darker) shows results for saline treatment.

FIG. 8 shows the total walking distance (in meters) of pigs following the intradermal administration of either saline or Dysport at 15 days (A), 5 days (B) or 1 day (C) prior to surgery.

FIG. 9 shows the time course of time spent in the central zone of an open-field apparatus in a period of 5 minutes (Individual values and Median) following the intradermal administration of either saline or Dysport at 15 days (A), 5 days (B) or 1 day (C) prior to surgery.

FIG. 10 shows a Von Frey assay of saline versus BoNT/A (Dysport) when administered via intradermal (A), subcutaneous (B) or intramuscular (C) injections. For each injection route tested, a total of 200U of Dysport per pig was administered.

FIG. 11 shows the latency (in seconds) of pigs to approach their handler when administered saline or BoNT/A (Dysport) via intradermal (A), subcutaneous (B) or intramuscular (C) injections. For each injection route tested, a total of 200U of Dysport per pig was administered.

FIG. 12 shows the distress behaviour score of pigs when administered saline or BoNT/A (Dysport) via intradermal (a), subcutaneous (b) or intramuscular (c) injections. For each injection route tested, a total of 200U of Dysport per pig was administered.

FIG. 13 shows the time course of total distance (in metres) that the animals walked in a period of 5 minutes in open-field following intradermal, subcutaneous or intramuscular administration of saline or Dysport (Individual values and Mean±SEM).

FIG. 14 shows the time course of time (percentage) spent in the central zone of an open-field apparatus following the intradermal, subcutaneous or intramuscular administration of saline or Dysport (Individual values and Mean±SEM).

FIG. 15 shows immunohistochemistry staining of SNAP-25 in skin samples with small nerves around arterioles (A), nerve endings in the hair erector muscles (B) and small-middle sized nerves in the dermis (C).

FIG. 16 shows immunohistochemistry staining of cleaved SNAP-25 in the spinal cord of a pig when untreated (A) and cleaved SNAP-25 staining in the ipsilateral horn (B) or contralateral horn (C) of a pig treated with Dysport.

FIG. 17 shows the expression levels of calcitonin gene related peptide (CGRP) and Substance P in the spinal cord of a pig when either untreated (A, C) or administered an intradermal injection of Dysport (B, D).

FIG. 18 shows the expression levels of Iba1 (A, B) and glial fibrillary acidic protein (GFAP) (C, D) in the spinal cord of a pig when untreated or when administered with intradermal injections of Dysport.

FIG. 19 shows a Von Frey assay of either saline (control) or BoNT/A (Dysport) intradermal administration at 15 days prior to surgery or Exparel intradermal administration on the day of surgery (D1), and when pigs are subjected to a surgical incision in the left leg (A) * p<0.05; ** p<0.01; *** p<0.001; ****p<0.0001 vs. saline group using one way ANOVA followed by Tukey test. #p<0.05; ##p<0.01 ####p<0.0001 Dysport vs. Exparel group using one way ANOVA followed by Tukey test. $$ p<0.01: post-surgery timepoint vs. Day −4 using paired T-test). (B) shows the latency (in seconds) of pigs to approach their handler following the intradermal administration of either saline (control) or BoNT/A (Dysport) intradermal administration at 15 days prior to surgery or Exparel intradermal administration on the day of surgery (D1), and when pigs are subjected to a surgical incision in the left leg ($$ p<0.01: post-surgery timepoint vs. Day −4 using paired T-test. £££p<0.001: Day −16 vs. Day −4 using paired T-test. *p<0.05; ** p<0.01; ***p<0.001 treatment vs. saline group using one-way ANOVA followed by Tukey test). (C) shows the distress behaviour score of pigs following the intradermal administration of either saline (control) or BoNT/A (Dysport) intradermal administration at 15 days prior to surgery or Exparel intradermal administration on the day of surgery (D1), and when pigs are subjected to a surgical incision in the left leg (*p<0.05, ***p<0.001 and ****p<0.0001 vs. saline group using one way ANOVA followed by Tukey test. $$ p<0.01, $p<0.05: post-surgery timepoint vs. Day −4 using paired T-test).

FIG. 20 shows a summary of the tissue samples (formalin-fixed-paraffin-embedded tissues) that were collected for immunohistochemistry staining and the specific regions where tissue samples were collected from the spinal cord.

FIG. 21 shows the immunohistochemistry staining for cleaved SNAP-25 in the skin (A), muscle (B), and dorsal root ganglia (C) in pigs with a surgical incision in the left leg.

FIG. 22 shows cleaved SNAP-25 staining in the ipsilateral dorsal horn of lumbar L5-L6 in the spinal cord of pigs with a surgical incision to the left leg (A) and a magnified view (B).

FIG. 23 shows a grading scale used to determine the intensity of cleaved SNAP-25 staining. Cleaved SNAP-25 staining was graded on a scale of 1-3, with grade 0=no cleaved SNAP-25 staining, grade 1=low intensity cleaved SNAP-25 staining, grade 2=average cleaved SNAP-25 intensity staining and grade 3=high intensity cleaved SNAP-25 staining (A). FIG. 23 also shows the quantification of staining intensity for different regions of the spinal cord: lumbar L5-L6, L3-L4, L1-L2 and the thoracic and cervical regions. Staining intensity was measured by way of a “H-Score” and calculated by % of positive spinal cord sections x staining intensity in the dorsal horns (B).

FIG. 24 provides a summary of the presence, “positive” or absence, “negative” of cleaved SNAP-25 staining in collected tissue samples.

SEQUENCE LISTING

Where an initial Met amino acid residue or a corresponding initial codon is indicated in
any of the following SEQ ID NOs, said residue/codon is optional.
SEQ ID NO: 1-BoNT/A1, accession number A5HZZ9, amino acid seauence
MPFVNKQFNYKDPVNGVDIAYIKIPNAGQMQPVKAFKIHNKIWVIPERDTFTNPEEGDLNPPPEAKQVPVSYYDSTYLST
DNEKDNYLKGVTKLFERIYSTDLGRMLLTSIVRGIPFWGGSTIDTELKVIDTNCINVIQPDGSYRSEELNLVIIGPSADI
IQFECKSFGHEVLNLTRNGYGSTQYIRFSPDFTFGFEESLEVDTNPLLGAGKFATDPAVTLAHELIHAGHRLYGIAINPN
RVFKVNTNAYYEMSGLEVSFEELRTFGGHDAKFIDSLQENEFRLYYYNKFKDIASTLNKAKSIVGTTASLQYMKNVFKEK
YLLSEDTSGKFSVDKLKFDKLYKMLTEPYTEDNFVKFFKVLNRKTYLNFDKAVFKPNPVPKVNYTPYDGFNLRNTNPAAN
FNGQNTEINNMNFTKLKNFTGLFEFYKLLCVRGIITSKTKSLDKGYNKALNDLCIKVNNWDLFFSPSEDNFTNDLNKGEE
ITSDTNIEAAEENISLDLIQQYYLTFNFDNEPENISIENLSSDIIGQLELMPNIERFPNGKKYELDKYTMFHYLRAQEFE
HGKSRIALTNSVNEALLNPSRVYTFFSSDYVKKVNKATEAAMFLGWVEQLVYDFTDETSEVSTTDKIADITIIIPYIGPA
LNIGNMLYKDDFVGALIFSGAVILLEFIPEIAIPVLGTFALVSYIANKVLTVQTIDNALSKRNEKWDEVYKYIVTNWLAK
VNTQIDLIRKKMKEALENQAEATKAIINYQYNQYTEEEKNNINFNIDDLSSKLNESINKAMININKFLNQCSVSYLMNSM
PPYGVKRLEDFDASLKDALLKYPYDNRGTLPGQVDRLKDKVNNTLSTDPPFQLSKYVDNQRLLSTFTEYPKNPPNTSPLN
LRYESNHLIDLSRYASKINIGSKVNFDPIDKNQIQLFNLESSKIEVILKNAIVYNSMYENFSTSFWIRIPKYFNSISLNN
EYTIINCMENNSGWKVSLNYGEIIWTLQDTQEIKQRVVFKYSQMINISDYINRWIFVTITNNRLNNSKIYINGRLIDQKP
PSNLGNPHASNNPMFKLDGCRDTHRYPWPKYFNLFDKELNEKEPKDLYDNQSNSGPLKDFWGDYLQYDKPYYMLNLYDPN
KYVDVNNVGIRGYMYLKGPRGSVMTTNIYLNSSLYRGTKFIIKKYASGNKDNIVRNNDRVYINVVVKNKEYRLATNASQA
GVEKILSALEIPDVGNLSQVVVMKSKNDQGITNKCKMNLQDNNGNDIGFIGFHQFNNIAKLVASNWYNRQIERSSRTLGC
SWEEPPVDDGWGERPL
SEQ ID NO: 2-BoNT/B1, accession number B1INP5, amino acid seauence
MPVTPNNFNYNDPPDNNNPPMMEPPFARGTGRYYKAFKPTDRPWPPPERYTFGYKPEDFNKSSGPFNRDVCEYYDPDYLN
TNDKKNIFLQTMIKLFNRIKSKPLGEKLLEMIINGIPYLGDRRVPLEEFNTNIASVTVNKLISNPGEVERKKGIFANLII
FGPGPVLNENETPDPGPQNHFASREGFGGPMQMKFCPEYVSVFNNVQENKGASPFNRRGYFSDPALPLMHELPHVLHGLY
GIKVDDLPIVPNEKKFFMQSTDAIQAEELYTFGGQDPSIITPSTDKSIYDKVLQNFRGIVDRLNKVLVCISDPNININIY
KNKFKDKYKFVEDSEGKYSPDVESFDKLYKSLMFGFTETNPAENYKPKTRASYFSDSLPPVKPKNLLDNEPYTPEEGFNP
SDKDMEKEYRGQNKAINKQAYEEISKEHLAVYKIQMCKSVKAPGICIDVDNEDLFFIADKNSFSDDLSKNERIEYNTQSN
YPENDFPPNELPLDTDLPSKPELPSENTESLTDFNVDVPVYEKQPAPKKPFTDENTPFQYLYSQTFPLDPRDPSLTSSFD
DALLFSNKVYSFFSMDYPKTANKVVEAGLFAGWVKQPVNDFVPEANKSNTMDKPADPSLPVPYPGLALNVGNETAKGNFE
NAFEIAGASILLEFIPELLIPVVGAFLLESYIDNKNKIIKTIDNALTKRNEKWSDMYGLIVAQWLSTVNTQFYTIKEGMY
KALNYQAQALEEPPKYRYNPYSEKEKSNPNPDFNDPNSKLNEGPNQAPDNPNNFPNGCSVSYLMKKMPPLAVEKLLDFDN
TLKKNLLNYPDENKLYLPGSAEYEKSKVNKYLKTPMPFDLSPYTNDTPLPEMFNKYNSEPLNNPPLNLRYKDNNLPDLSG
YGAKVEVYDGVELNDKNQFKLTSSANSKPRVTQNQNPPFNSVFLDFSVSFWPRPPKYKNDGPQNYPHNEYTPPNCMKNNS
GWKISIRGNRIIWTLIDINGKTKSVFFEYNIREDISEYINRWFFVTITNNLNNAKIYINGKLESNTDIKDIREVIANGEI
IFKLDGDIDRTQFIWMKYFSIFNTELSQSNIEERYKIQSYSEYLKDFWGNPLMYNKEYYMFNAGNKNSYIKLKKDSPVGE
ILTRSKYNQNSKYINYRDLYIGEKFIIRRKSNSQSINDDIVRKEDYIYLDFFNLNQEWRVYTYKYFKKEEEKLFLAPISD
SDEFYNTPQPKEYDEQPTYSCQLLFKKDEESTDEPGLPGPHRFYESGPVFEEYKDYFCPSKWYLKEVKRKPYNLKLGCNW
QFPPKDEGWTE
SEQ ID NO: 3-BoNT/C1, accession number P18640, amino acid seauence
MPITINNFNYSDPVDNKNILYLDTHLNTLANEPEKAFRITGNIWVIPDRFSRNSNPNLNKPPRVTSPKSGYYDPNYLSTD
SDKDPFLKEIIKLFKRINSREIGEELIYRLSTDIPFPGNNNTPINTFDFDVDFNSVDVKTRQGNNWVKTGSINPSVIITG
PRENIIDPETSTFKLTNNTFAAQEGFGALSIISISPRFMLTYSNATNDVGEGRFSKSEFCMDPILILMHELNHAMHNLYG
PAIPNDQTPSSVTSNPFYSQYNVKLEYAEPYAFGGPTPDLPPKSARKYFEEKALDYYRSPAKRLNSPTTANPSSFNKYPG
EYKQKLIRKYRFVVESSGEVTVNRNKFVELYNELTQIFTEFNYAKIYNVQNRKIYLSNVYTPVTANILDDNVYDIQNGFN
PPKSNLNVLFMGQNLSRNPALRKVNPENMLYLFTKFCHKAPDGRSLYNKTLDCRELLVKNTDLPFPGDPSDVKTDPFLRK
DPNEETEVPYYPDNVSVDQVPLSKNTSEHGQLDLLYPSPDSESEPLPGENQVFYDNRTQNVDYLNSYYYLESQKLSDNVE
DFTFTRSIEEALDNSAKVYTYFPTLANKVNAGVQGGLFLMWANDVVEDFTTNILRKDTLDKISDVSAIIPYIGPALNISN
SVRRGNFTEAFAVTGVTPLLEAFPEFTPPALGAFVPYSKVQERNEPPKTPDNCLEQRPKRWKDSYEWMMGTWLSRPPTQF
NNPSYQMYDSLNYQAGAPKAKPDLEYKKYSGSDKENPKSQVENLKNSLDVKPSEAMNNPNKFPRECSVTYLFKNMLPKVP
DELNEFDRNTKAKLPNLPDSHNPPLVGEVDKLKAKVNNSFQNTPPFNPFSYTNNSLLKDPPNEYFNNPNDSKPLSLQNRK
NTLVDTSGYNAEVSEEGDVQLNPPFPFDFKLGSSGEDRGKVPVTQNENPVYNSMYESFSPSFWPRPNKWVSNLPGYTPPD
SVKNNSGWSIGIISNFLVFTLKQNEDSEQSINFSYDISNNAPGYNKWFFVTVTNNMMGNMKIYINGKLIDTIKVKELTGI
NFSKTITFEINKIPDTGLITSDSDNINMWIRDFYIFAKELDGKDINILFNSLQYTNVVKDYWGNDLRYNKEYYMVNIDYL
NRYMYANSRQPVFNTRRNNNDFNEGYKPPPKRPRGNTNDTRVRGGDPLYFDMTPNNKAYNLFMKNETMYADNHSTEDPYA
PGLREQTKDPNDNPPFQPQPMNNTYYYASQPFKSNFNGENPSGPCSPGTYRFRLGGDWYRHNYLVPTVKQGNYASLLEST
STHWGFVPVSE
SEQ ID NO: 4-BoNT/D, accession number P19321, amino acid seauence
MTWPVKDFNYSDPVNDNDILYLRIPQNKLITTPVKAFMITQNIWVIPERFSSDTNPSLSKPPRPTSKYQSYYDPSYLSTD
EQKDTFLKGIIKLFKRINERDIGKKLINYLVVGSPFMGDSSTPEDTFDFTRHTTNIAVEKFENGSWKVTNIITPSVLIFG
PLPNILDYTASLTLQGQQSNPSFEGFGTLSILKVAPEFLLTFSDVTSNQSSAVLGKSIFCMDPVIALMHELTHSLHQLYG
INIPSDKRIRPQVSEGFFSQDGPNVQFEELYTFGGLDVEIIPQIERSQLREKALGHYKDIAKRLNNINKTIPSSWISNID
KYKKIFSEKYNFDKDNTGNFVVNIDKFNSLYSDLTNVMSEVVYSSQYNVKNRTHYFSRHYLPVFANILDDNIYTIRDGFN
LTNKGFNIENSGQNIERNPALQKLSSESVVDLETKVCLRLTKNSRDDSTCIKVKNNRLPYVADKDSISQEIFENKIITDE
TNVQNYSDKFSLDESILDGQVPINPEIVDPLLPNVNMEPLNLPGEEIVFYDDITKYVDYLNSYYYLESQKLSNNVENITL
TTSVEEALGYSNKIYTFLPSLAEKVNKGVQAGLFLNWANEVVEDFTTNIMKKDTLDKISDVSVIIPYIGPALNIGNSALR
GNFNQAFATAGVAFLLEGFPEFTIPALGVFTFYSSIQEREKIIKTIENCLEQRVKRWKDSYQWMVSNWLSRITTQFNHIN
YQMYDSLSYQADAIKAKIDLEYKKYSGSDKENIKSQVENLKNSLDVKISEAMNNINKFIRECSVTYLFKNMLPKVIDELN
KFDLRTKTELINLIDSHNIILVGEVDRLKAKVNESFENTMPFNIFSYTNNSLLKDIINEYFNSINDSKILSLQNKKNALV
DTSGYNAEVRVGDNVQLNTIYTNDFKLSSSGDKIIVNLNNNILYSAIYENSSVSFWIKISKDLTNSHNEYTIINSIEQNS
GWKLCIRNGNIEWILQDVNRKYKSLIFDYSESLSHTGYTNKWFFVTITNNIMGYMKLYINGELKQSQKIEDLDEVKLDKT
IVFGIDENIDENQMLWIRDFNIFSKELSNEDINIVYEGQILRNVIKDYWGNPLKFDTEYYIINDNYIDRYIAPESNVLVL
VQYPDRSKLYTGNPITIKSVSDKNPYSRILNGDNIILHMLYNSRKYMIIRDTDTIYATQGGECSQNCVYALKLQSNLGNY
GIGIFSIKNIVSKNKYCSQIFSSFRENTMLLADIYKPWRFSFKNAYTPVAVTNYETKLLSTSSFWKFISRDPGWVE
SEQ ID NO: 5-BoNT/E1, accession number WP_003372387, amino acid sequence
MPKINSFNYNDPVNDRTILYIKPGGCQEFYKSFNIMKNIWIIPERNVIGTTPQDFHPPTSLKNGDSSYYDPNYLQSDEEK
DRFLKIVTKIFNRINNNLSGGILLEELSKANPYLGNDNTPDNQFHIGDASAVEIKFSNGSQDILLPNVIIMGAEPDLFET
NSSNISLRNNYMPSNHGFGSIAIVTFSPEYSFRFNDNSMNEFIQDPALTLMHELIHSLHGLYGAKGITTKYTITQKQNPL
ITNIRGTNIEEFLTFGGTDLNIITSAQSNDIYTNLLADYKKIASKLSKVQVSNPLLNPYKDVFEAKYGLDKDASGIYSVN
INKFNDIFKKLYSFTEFDLATKFQVKCRQTYIGQYKYFKLSNLLNDSIYNISEGYNINNLKVNFRGQNANLNPRIITPIT
GRGLVKKIIRFCKNIVSVKGIRKSICIEINNGELFFVASENSYNDDNINTPKEIDDTVTSNNNYENDLDQVILNFNSESA
PGLSDEKLNLTIQNDAYIPKYDSNGTSDIEQHDVNELNVFFYLDAQKVPEGENNVNLTSSIDTALLEQPKIYTFFSSEFI
NNVNKPVQAALFVSWIQQVLVDFTTEANQKSTVDKIADISIVVPYIGLALNIGNEAQKGNFKDALELLGAGILLEFEPEL
LIPTILVFTIKSFLGSSDNKNKVIKAINNALKERDEKWKEVYSFIVSNWMTKINTQFNKRKEQMYQALQNQVNAIKTIIE
SKYNSYTLEEKNELTNKYDIKQIENELNQKVSIAMNNIDRFLTESSISYLMKLINEVKINKLREYDENVKTYLLNYIIQH
GSILGESQQELNSMVTDTLNNSIPFKLSSYTDDKILISYFNKFFKRIKSSSVLNMRYKNDKYVDTSGYDSNININGDVYK
YPTNKNQFGIYNDKLSEVNISQNDYIIYDNKYKNFSISFWVRIPNYDNKIVNVNNEYTIINCMRDNNSGWKVSLNHNEII
WTLQDNAGINQKLAFNYGNANGISDYINKWIFVTITNDRLGDSKLYINGNLIDQKSILNLGNIHVSDNILFKIVNCSYTR
YIGIRYFNIFDKELDETEIQTLYSNEPNTNILKDFWGNYLLYDKEYYLLNVLKPNNFIDRRKDSTLSINNIRSTILLANR
LYSGIKVKIQRVNNSSTNDNLVRKNDQVYINFVASKTHLFPLYADTATTNKEKTIKISSSGNRFNQVVVMNSVGNNCTMN
FKNNNGNNIGLLGFKADTVVASTWYYTHMRDHTNSNGCFWNFISEEHGWQEK
SEQ ID NO: 6-BoNT/F1, accession number Q57236, amino acid sequence
MPVVINSFNYNDPVNDDTILYMQIPYEEKSKKYYKAFEIMRNVWIIPERNTIGTDPSDFDPPASLENGSSAYYDPNYLTT
DAEKDRYLKTTIKLFKRINSNPAGEVLLQEISYAKPYLGNEHTPINEFHPVTRTTSVNIKSSTNVKSSIILNLLVLGAGP
DIFENSSYPVRKLMDSGGVYDPSNDGFGSINIVTFSPEYEYTFNDISGGYNSSTESFIADPAISLAHELIHALHGLYGAR
GVTYKETIKVKQAPLMIAEKPIRLEEFLTFGGQDLNIITSAMKEKIYNNLLANYEKIATRLSRVNSAPPEYDINEYKDYF
QWKYGLDKNADGSYTVNENKFNEIYKKLYSFTEIDLANKFKVKCRNTYFIKYGFLKVPNLLDDDIYTVSEGFNIGNLAVN
NRGQNIKLNPKIIDSIPDKGLVEKIVKFCKSVIPRKGTKAPPRLCIRVNNRELFFVASESSYNENDINTPKEIDDTTNLN
NNYRNNLDEVILDYNSETIPQISNQTLNTLVQDDSYVPRYDSNGTSEIEEHNVVDLNVFFYLHAQKVPEGETNISLTSSI
DTALSEESQVYTFFSSEFINTINKPVHAALFISWINQVIRDFTTEATQKSTFDKIADISLVVPYVGLALNIGNEVQKENF
KEAFELLGAGILLEFVPELLIPTILVFTIKSFIGSSENKNKIIKAINNSLMERETKWKEIYSWIVSNWLTRINTQFNKRK
EQMYQALQNQVDAIKTVIEYKYNNYTSDERNRLESEYNINNIREELNKKVSLAMENIERFITESSIFYLMKLINEAKVSK
LREYDEGVKEYLLDYISEHRSILGNSVQELNDLVTSTLNNSIPFELSSYTNDKILILYFNKLYKKIKDNSILDMRYENNK
FIDISGYGSNISINGDVYIYSTNRNQFGIYSSKPSEVNIAQNNDIIYNGRYQNFSISFWVRIPKYFNKVNLNNEYTIIDC
IRNNNSGWKISLNYNKIIWTLQDTAGNNQKLVFNYTQMISISDYINKWIFVTITNNRLGNSRIYINGNLIDEKSISNLGD
IHVSDNILFKIVGCNDTRYVGIRYFKVFDTELGKTEIETLYSDEPDPSILKDFWGNYLLYNKRYYLLNLLRTDKSITQNS
NFLNINQQRGVYQKPNIFSNTRLYTGVEVIIRKNGSTDISNTDNFVRKNDLAYINVVDRDVEYRLYADISIAKPEKIIKL
IRTSNSNNSLGQIIVMDSIGNNCTMNFQNNNGGNIGLLGFHSNNLVASSWYYNNIRKNTSSNGCFWSFISKEHGWQEN
SEQ ID NO: 7-BoNT/G, accession number WP_039635782, amino acid sequence
MPVNIKNFNYNDPINNDDIIMMEPFNDPGPGTYYKAFRIIDRIWIVPERFTYGFQPDQFNASTGVFSKDVYEYYDPTYLK
TDAEKDKFLKTMIKLFNRINSKPSGQRLLDMIVDAIPYLGNASTPPDKFAANVANVSINKKIIQPGAEDQIKGLMTNLII
FGPGPVLSDNFTDSMIMNGHSPISEGFGARMMIRFCPSCLNVFNNVQENKDTSIFSRRAYFADPALTLMHELIHVLHGLY
GIKISNLPITPNTKEFFMQHSDPVQAEELYTFGGHDPSVISPSTDMNIYNKALQNFQDIANRLNIVSSAQGSGIDISLYK
QIYKNKYDFVEDPNGKYSVDKDKFDKLYKALMFGFTETNLAGEYGIKTRYSYFSEYLPPIKTEKLLDNTIYTQNEGFNIA
SKNLKTEFNGQNKAVNKEAYEEISLEHLVIYRIAMCKPVMYKNTGKSEQCIIVNNEDLFFIANKDSFSKDLAKAETIAYN
TQNNTIENNFSIDQLILDNDLSSGIDLPNENTEPFTNFDDIDIPVYIKQSALKKIFVDGDSLFEYLHAQTFPSNIENLQL
TNSLNDALRNNNKVYTFFSTNLVEKANTVVGASLFVNWVKGVIDDFTSESTQKSTIDKVSDVSIIIPYIGPALNVGNETA
KENFKNAFEIGGAAILMEFIPELIVPIVGFFTLESYVGNKGHIIMTISNALKKRDQKWTDMYGLIVSQWLSTVNTQFYTI
KERMYNALNNQSQAIEKIIEDQYNRYSEEDKMNINIDFNDIDFKLNQSINLAINNIDDFINQCSISYLMNRMIPLAVKKL
KDFDDNLKRDLLEYIDTNELYLLDEVNILKSKVNRHLKDSIPFDLSLYTKDTILIQVFNNYISNISSNAILSLSYRGGRL
IDSSGYGATMNVGSDVIFNDIGNGQFKLNNSENSNITAHQSKFVVYDSMFDNFSINFWVRTPKYNNNDIQTYLQNEYTII
SCIKNDSGWKVSIKGNRIIWTLIDVNAKSKSIFFEYSIKDNISDYINKWFSITITNDRLGNANIYINGSLKKSEKILNLD
RINSSNDIDFKLINCTDTTKFVWIKDFNIFGRELNATEVSSLYWIQSSTNTLKDFWGNPLRYDTQYYLFNQGMQNIYIKY
FSKASMGETAPRTNFNNAAINYQNLYLGLRFIIKKASNSRNINNDNIVREGDYIYLNIDNISDESYRVYVLVNSKEIQTQ
LFLAPINDDPTFYDVLQIKKYYEKTTYNCQILCEKDTKTFGLFGIGKFVKDYGYVWDTYDNYFCISQWYLRRISENINKL
RLGCNWQFI PVDEGWTE
SEQ ID NO: 8-BoNT/DC, accession number BAM65681, amino acid sequence
MTWPVKDFNYSDPVNDNDILYLRIPQNKLITTPVKAFMITQNIWVIPERFSSDTNPSLSKPPRPTSKYQSYYDPSYLSTD
EQKDTFLKGIIKLFKRINERDIGKKLINYLVVGSPFMGDSSTPEDTFDFTRHTTNIAVEKFENGSWKVTNIITPSVLIFG
PLPNILDYTASLTLQGQQSNPSFEGFGTLSILKVAPEFLLTFSDVTSNQSSAVLGKSIFCMDPVIALMHELTHSLHQLYG
INIPSDKRIRPQVSEGFFSQDGPNVQFEELYTFGGSDVEIIPQIERLQLREKALGHYKDIAKRLNNINKTIPSSWSSNID
KYKKIFSEKYNFDKDNTGNFVVNIDKFNSLYSDLTNVMSEVVYSSQYNVKNRTHYFSKHYLPVFANILDDNIYTIINGFN
LTTKGFNIENSGQNIERNPALQKLSSESVVDLFTKVCLRLTRNSRDDSTCIQVKNNTLPYVADKDSISQEIFESQIITDE
TNVENYSDNFSLDESILDAKVPTNPEAVDPLLPNVNMEPLNVPGEEEVFYDDITKDVDYLNSYYYLEAQKLSNNVENITL
TTSVEEALGYSNKIYTFLPSLAEKVNKGVQAGLFLNWANEVVEDFTTNIMKKDTLDKISDVSAIIPYIGPALNIGNSALR
GNFKQAFATAGVAFLLEGFPEFTIPALGVFTFYSSIQEREKIIKTIENCLEQRVKRWKDSYQWMVSNWLSRITTQFNHIS
YQMYDSLSYQADAIKAKIDLEYKKYSGSDKENIKSQVENLKNSLDVKISEAMNNINKFIRECSVTYLFKNMLPKVIDELN
KFDLKTKTELINLIDSHNIILVGEVDRLKAKVNESFENTIPFNIFSYTNNSLLKDMINEYFNSINDSKILSLQNKKNTLM
DTSGYNAEVRVEGNVQLNPIFPFDFKLGSSGDDRGKVIVTQNENIVYNAMYESFSISFWIRINKWVSNLPGYTIIDSVKN
NSGWSIGIISNFLVFTLKQNENSEQDINFSYDISKNAAGYNKWFFVTITTNMMGNMMIYINGKLIDTIKVKELTGINFSK
TITFQMNKIPNTGLITSDSDNINMWIRDFYIFAKELDDKDINILFNSLQYTNVVKDYWGNDLRYDKEYYMINVNYMNRYM
SKKGNGIVFNTRKNNNDFNEGYKIIIKRIIGNTNDTRVRGENVLYFNTTIDNKQYSLGMYKPSRNLGTDLVPLGALDQPM
DEIRKYGSFIIQPCNTFDYYASQLFLSSNATTNRIGILSIGSYSFKLGDDYWFNHEYLIPVIKIEHYASLLESTSTHWVF
VPASE
SEQ ID NO: 9-BoNT/F7, amino acid sequence
MPVNINNFNYNDPINNTTILYMKMPYYEDSNKYYKAFEIMDNVWIIPERNIIGKKPSDFYPPISLDSGSSAYYDPNYLTT
DAEKDRFLKTVIKLFNRINSNPAGQVLLEEIKNGKPYLGNDHTAVNEFCANNRSTSVEIKESKGTTDSMLLNLVILGPGP
NILECSTFPVRIFPNNIAYDPSEKGFGSIQLMSFSTEYEYAFNDNTDLFIADPAISLAHELIHVLHGLYGAKGVTNKKVI
EVDQGALMAAEKDIKIEEFITFGGQDLNIITNSTNQKIYDNLLSNYTAIASRLSQVNINNSALNTTYYKNFFQWKYGLDQ
DSNGNYTVNISKFNAIYKKLFSFTECDLAQKFQVKNRSNYLFHFKPFRLLDLLDDNIYSISEGFNIGSLRVNNNGQNINL
NSRIVGPIPDNGLVERFVGLCKSIVSKKGTKNSLCIKVNNRDLFFVASESSYNENGINSPKEIDDTTITNNNYKKNLDEV
ILDYNSDAIPNLSSRLLNTTAQNDSYVPKYDSNGTSEIKEYTVDKLNVFFYLYAQKAPEGESAISLTSSVNTALLDASKV
YTFFSSDFINTVNKPVQAALFISWIQQVINDFTTEATQKSTIDKIADISLVVPYVGLALNIGNEVQKGNFKEAIELLGAG
ILLEFVPELLIPTILVFTIKSFINSDDSKNKIIKAINNALRERELKWKEVYSWIVSNWLTRINTQFNKRKEQMYQALQNQ
VDGIKKIIEYKYNNYTLDEKNRLKAEYNIYSIKEELNKKVSLAMQNIDRFLTESSISYLMKLINEAKINKLSEYDKRVNQ
YLLNYILENSSTLGTSSVQELNNLVSNTLNNSIPFELSEYTNDKILISYFNRFYKRIIDSSILNMKYENNRFIDSSGYGS
NISINGDIYIYSTNRNQFGIYSSRLSEVNITQNNTIIYNSRYQNFSVSFWVRIPKYNNLKNLNNEYTIINCMRNNNSGWK
ISLNYNNIIWTLQDTTGNNQKLVFNYTQMIDISDYINKWTFVTITNNRLGHSKLYINGNLTDQKSILNLGNIHVDDNILF
KIVGCNDTRYVGIRYFKIFNMELDKTEIETLYHSEPDSTILKDFWGNYLLYNKKYYLLNLLKPNMSVTKNSDILNINRQR
GIYSKTNIFSNARLYTGVEVIIRKVGSTDTSNTDNFVRKNDTVYINVVDGNSEYQLYADVSTSAVEKTIKLRRISNSNYN
SNQMIIMDSIGDNCTMNFKTNNGNDIGLLGFHLNNLVASSWYYKNIRNNTRNNGCFWSFISKEHGWQE
SEQ ID NO: 10-BoNT/ABmy, amino acid sequence

| | |
|---|---|
| MPFVNKQFNYKDPVNGVDIAYIKIPNAGQMQPVKAFKIHNKIWVIPERD | 49 |
| TFTNPEEGDLNPPPEAKQVPVSYYDSTYLSTDNEKDNYLKGVTKLFERIY | 99 |
| STDLGRMLLTSIVRGIPFWGGSTIDTELKVIDTNCINVIQPDGSYRSEEL | 149 |
| NLVIIGPSADIIQFECKSFGHEVLNLTRNGYGSTQYIRFSPDFTFGFEES | 199 |
| LEVDTNPLLGAGKFATDPAVTLAHELIHAGHRLYGIAINPNRVFKVNTNA | 249 |
| YYEMSGLEVSFEELRTFGGHDAKFIDSLQENEFRLYYYNKFKDIASTLNK | 299 |
| AKSIVGTTASLQYMKNVFKEKYLLSEDTSGKFSVDKLKFDKLYKMLTEIY | 349 |
| TEDNFVKFFKVLNRKTYLNFDKAVFKINIVPKVNYTIYDGFNLRNTNLAA | 399 |
| NFNGQNTEINNMNFTKLKNFTGLFEFYKLLCVRGIITSKTKSLDKGYNKA | 449 |
| LNDLCIKVNNWDLFFSPSEDNFTNDLNKGEEITSDTNIEAAEENISLDLI | 499 |
| QQYYLTFNFDNEPENISIENLSSDIIGQLELMPNIERFPNGKKYELDKYT | 549 |
| MFHYLRAQEFEHGKSRIALTNSVNEALLNPSRVYTFFSSDYVKKVNKATE | 599 |
| AAMFLGWVEQLVYDFTDETSEVSTTDKIADITIIIPYIGPALNIGNMLYK | 649 |
| DDFVGALIFSGAVILLEFIPEIAIPVLGTFALVSYIANKVLTVQTIDNAL | 699 |
| SKRNEKWDEVYKYIVTNWLAKVNTQIDLIRKKMKEALENQAEATKAIINY | 749 |
| QYNQYTEEEKNNINFNIDDLSSKLNESINKAMININKFLNQCSVSYLMNS | 799 |
| MIPYGVKRLEDFDASLKDALLKYIYDNRGTLIGQVDRLKDKVNNTLSTDI | 849 |
| PFQLSKYVDNQRLLSTFTEYIKNILNNIILNLRYKDNNLIDLSGYGAKVE | 899 |
| VYDGVELNDKNQFKLTSSANSKIRVTQNQNIIFNSVFLDFSVSFWIRIPK | 949 |
| YKNDGIQNYIHNEYTIINCMKNNSGWKISIRGNRIIWTLIDINGKTKSVF | 999 |
| FEYNIREDISEYINRWFFVTITNNLNNAKIYINGKLESNTDIKDIREVIA | 1049 |
| NGEIIFKLDGDIDRTQFIWMKYFSIFNTELSQSNIEERYKIQSYSEYLKD | 1099 |
| FWGNPLMYNKEYYMFNAGNKNSYIKLKKDSPVGEILTRSKYNQNSKYINY | 1149 |
| RDLYIGEKFIIRRKSNSQSINDDIVRKEDYIYLDFFNLNQEWRVYTYKYF | 1199 |
| KKEEMKLFLAPIYDSDEFYNTIQIKEYDEQPTYSCQLLFKKDEESTDEIG | 1249 |
| LIGIHRFYESGIVFEEYKDYFCISKWYLKEVKRKPYNLKLGCNWQFIPKD | 1299 |
| EGWTE | 1304 |

SEQ ID NO: 11-BoNT/X, amino acid sequence (GenBank: BAQ12790.1)
MKLEINKFNYNDPIDGINVITMRPPRHSDKINKGKGPFKAFQVIKNIWIVPERYNFTNNTNDLNIPSEPIMEADAIYNPNYLN
TPSEKDEFLQGVIKVLERIKSKPEGEKLLELISSSIPLPLVSNGALTLSDNETIAYQENNNIVSNLQANLVIYGPGPDIANNA
TYGLYSTPISNGEGTLSEVSFSPFYLKPFDESYGNYRSLVNIVNKFVKREFAPDPASTLMHELVHVTHNLYGISNRNFYYNFD
TGKIETSRQQNSLIFEELLTFGGIDSKAISSLIIKKIIETAKNNYTTLISERLNTVTVENDLLKYIKNKIPVQGRLGNFKLDT
AEFEKKLNTILFVLNESNLAQRFSILVRKHYLKERPIDPIYVNILDDNSYSTLEGFNISSQGSNDFQGQLLESSYFEKIESNA
LRAFIKICPRNGLLYNAIYRNSKNYLNNIDLEDKKTTSKTNVSYPCSLLNGCIEVENKDLFLISNKDSLNDINLSEEKIKPET
TVFFKDKLPPQDITLSNYDFTEANSIPSISQQNILERNEELYEPIRNSLFEIKTIYVDKLTTFHFLEAQNIDESIDSSKIRVE
LTDSVDEALSNPNKVYSPFKNMSNTINSIETGITSTYIFYQWLRSIVKDFSDETGKIDVIDKSSDTLAIVPYIGPLLNIGNDI
RHGDFVGAIELAGITALLEYVPEFTIPILVGLEVIGGELAREQVEAIVNNALDKRDQKWAEVYNITKAQWWGTIHLQINTRLA
HTYKALSRQANAIKMNMEFQLANYKGNIDDKAKIKNAISETEILLNKSVEQAMKNTEKFMIKLSNSYLTKEMIPKVQDNLKNF
DLETKKTLDKFIKEKEDILGTNLSSSLRRKVSIRLNKNIAFDINDIPFSEFDDLINQYKNEIEDYEVLNLGAEDGKIKDLSGT
TSDINIGSDIELADGRENKAIKIKGSENSTIKIAMNKYLRFSATDNFSISFWIKHPKPTNLLNNGIEYTLVENFNQRGWKISI
QDSKLIWYLRDHNNSIKIVTPDYIAFNGWNLITITNNRSKGSIVYVNGSKIEEKDISSIWNTEVDDPIIFRLKNNRDTQAFTL
LDQFSIYRKELNQNEVVKLYNYYFNSNYIRDIWGNPLQYNKKYYLQTQDKPGKGLIREYWSSFGYDYVILSDSKTITFPNNIR
YGALYNGSKVLIKNSKKLDGLVRNKDFIQLEIDGYNMGISADRFNEDTNYIGTTYGTTHDLTTDFEIIQRQEKYRNYCQLKTP
YNIFHKSGLMSTETSKPTFHDYRDWVYSSAWYFQNYENLNLRKHTKTNWYFIPKDEGWDED
SEQ ID NO: 12 (Nucleotide Sequence of Unmodified BoNT/A)
ATGCCATTCGTCAACAAGCAATTCAACTACAAAGACCCAGTCAACGGCGTCGACATCGCATACATCAAGATTCCGAACGCCGG
TCAAATGCAGCCGGTTAAGGCTTTTAAGATCCACAACAAGATTTGGGTTATCCCGGAGCGTGACACCTTCACGAACCCGGAAG
AAGGCGATCTGAACCCGCCACCGGAAGCGAAGCAAGTCCCTGTCAGCTACTACGATTCGACGTACCTGAGCACGGATAACGAA
AAAGATAACTACCTGAAAGGTGTGACCAAGCTGTTCGAACGTATCTACAGCACGGATCTGGGTCGCATGCTGCTGACTAGCAT
TGTTCGCGGTATCCCGTTCTGGGGTGGTAGCACGATTGACACCGAACTGAAGGTTATCGACACTAACTGCATTAACGTTATTC
AACCGGATGGTAGCTATCGTAGCGAAGAGCTGAATCTGGTCATCATTGGCCCGAGCGCAGACATTATCCAATTCGAGTGCAAG
AGCTTTGGTCACGAGGTTCTGAATCTGACCCGCAATGGCTATGGTAGCACCCAGTACATTCGTTTTTCGCCGGATTTTACCTT
CGGCTTTGAAGAGAGCCTGGAGGTTGATACCAATCCGTTGCTGGGTGCGGGCAAATTCGCTACCGATCCGGCTGTCACGCTGG
CCCATGAACTGATCCACGCAGGCCACCGCCTGTACGGCATTGCCATCAACCCAAACCGTGTGTTCAAGGTTAATACGAATGCA
TACTACGAGATGAGCGGCCTGGAAGTCAGCTTCGAAGAACTGCGCACCTTCGGTGGCCATGACGCTAAATTCATTGACAGCTT
GCAAGAGAATGAGTTCCGTCTGTACTACTATAACAAATTCAAAGACATTGCAAGCACGTTGAACAAGGCCAAAAGCATCGTTG
GTACTACCGCGTCGTTGCAGTATATGAAGAATGTGTTTAAAGAGAAGTACCTGCTGTCCGAGGATACCTCCGGCAAGTTTAGC
GTTGATAAGCTGAAGTTTGACAAACTGTACAAGATGCTGACCGAGATTTACACCGAGGACAACTTTGTGAAATTCTTCAAAGT
GTTGAATCGTAAAACCTATCTGAATTTTGACAAAGCGGTTTTCAAGATTAACATCGTGCCGAAGGTGAACTACACCATCTATG
ACGGTTTTAACCTGCGTAACACCAACCTGGCGGCGAACTTTAACGGTCAGAATACGGAAATCAACAACATGAATTTCACGAAG
TTGAAGAACTTCACGGGTCTGTTCGAGTTCTATAAGCTGCTGTGCGTGCGCGGTATCATCACCAGCAAAACCAAAAGCCTGGA
CAAAGGCTACAACAAGGCGCTGAATGACCTGTGCATTAAGGTAAACAATTGGGATCTGTTCTTTTCGCCATCCGAAGATAATT
TTACCAACGACCTGAACAAGGGTGAAGAAATCACCAGCGATACGAATATTGAAGCAGCGGAAGAGAATATCAGCCTGGATCTG
ATCCAGCAGTACTATCTGACCTTTAACTTCGACAATGAACCGGAGAACATTAGCATTGAGAATCTGAGCAGCGACATTATCGG
TCAGCTGGAACTGATGCCGAATATCGAACGTTTCCCGAACGGCAAAAAGTACGAGCTGGACAAGTACACTATGTTCCATTACC
TGCGTGCACAGGAGTTTGAACACGGTAAAAGCCGTATCGCGCTGACCAACAGCGTTAACGAGGCCCTGCTGAACCCGAGCCGT
GTCTATACCTTCTTCAGCAGCGACTATGTTAAGAAAGTGAACAAAGCCACTGAGGCCGCGATGTTCCTGGGCTGGGTGGAACA
GCTGGTATATGACTTCACGGACGAGACGAGCGAAGTGAGCACTACCGACAAAATTGCTGATATTACCATCATTATCCCGTATA
TTGGTCCGGCACTGAACATTGGCAACATGCTGTACAAAGACGATTTTGTGGGTGCCCTGATCTTCTCCGGTGCCGTGATTCTG
CTGGAGTTCATTCCGGAGATTGCGATCCCGGTGTTGGGTACCTTCGCGCTGGTGTCCTACATCGCGAATAAGGTTCTGACGGT
TCAGACCATCGATAACGCGCTGTCGAAACGTAATGAAAAATGGGACGAGGTTTACAAATACATTGTTACGAATTGGCTGGCGA
AAGTCAATACCCAGATCGACCTGATCCGTAAGAAAATGAAAGAGGCGCTGGAGAATCAGGCGGAGGCCACCAAAGCAATTATC
AACTACCAATACAACCAGTACACGGAAGAAGAGAAGAATAACATTAACTTCAATATCGATGATTTGAGCAGCAAGCTGAATGA
ATCTATCAACAAAGCGATGATCAATATCAACAAGTTTTTGAATCAGTGTAGCGTTTCGTACCTGATGAATAGCATGATTCCGT
ATGGCGTCAAACGTCTGGAGGACTTCGACGCCAGCCTGAAAGATGCGTTGCTGAAATACATTTACGACAATCGTGGTACGCTG
ATTGGCCAAGTTGACCGCTTGAAAGACAAAGTTAACAATACCCTGAGCACCGACATCCCATTTCAACTGAGCAAGTATGTTGA
TAATCAACGTCTGTTGAGCACTTTCACCGAGTATATCAAAAACATCATCAATACTAGCATTCTGAACCTGCGTTACGAGAGCA
ATCATCTGATTGATCTGAGCCGTTATGCAAGCAAGATCAACATCGGTAGCAAGGTCAATTTTGACCCGATCGATAAGAACCAG
ATCCAGCTGTTTAATCTGGAATCGAGCAAAATTGAGGTTATCCTGAAAAACGCCATTGTCTACAACTCCATGTACGAGAATTT
CTCCACCAGCTTCTGGATTCGCATCCCGAAATACTTCAACAGCATTAGCCTGAACAACGAGTATACTATCATCAACTGTATGG
AGAACAACAGCGGTTGGAAGGTGTCTCTGAACTATGGTGAGATCATTTGGACCTTGCAGGACACCCAAGAGATCAAGCAGCGC
GTCGTGTTCAAGTACTCTCAAATGATCAACATTTCCGATTACATTAATCGTTGGATCTTCGTGACCATTACGAATAACCGTCT
GAATAACAGCAAGATTTACATCAATGGTCGCTTGATCGATCAGAAACCGATTAGCAACCTGGGTAATATCCACGCAAGCAACA
ACATTATGTTCAAATTGGACGGTTGCCGCGATACCCATCGTTATATCTGGATCAAGTATTTCAACCTGTTTGATAAAGAACTG
AATGAGAAGGAGATCAAAGATTTGTATGACAACCAATCTAACAGCGGCATTTTGAAGGACTTCTGGGGCGATTATCTGCAATA
CGATAAGCCGTACTATATGCTGAACCTGTATGATCCGAACAAATATGTGGATGTCAATAATGTGGGTATTCGTGGTTACATGT
ATTTGAAGGGTCCGCGTGGCAGCGTTATGACGACCAACATTTACCTGAACTCTAGCCTGTACCGTGGTACGAAATTCATCATT
AAGAAATATGCCAGCGGCAACAAAGATAACATTGTGCGTAATAACGATCGTGTCTACATCAACGTGGTCGTGAAGAATAAAGA
GTACCGTCTGGCGACCAACGCTTCGCAGGCGGGTGTTGAGAAAATTCTGAGCGCGTTGGAGATCCCTGATGTCGGTAATCTGA
GCCAAGTCGTGGTTATGAAGAGCAAGAACGACCAGGGTATCACTAACAAGTGCAAGATGAACCTGCAAGACAACAATGGTAAC
GACATCGGCTTTATTGGTTTCCACCAGTTCAACAATATTGCTAAACTGGTAGCGAGCAATTGGTACAATCGTCAGATTGAGCG
CAGCAGCCGTACTTTGGGCTGTAGCTGGGAGTTTATCCCGGTCGATGATGGTTGGGGCGAACGTCCGCTG
SEQ ID NO: 13 (Polypeptide Sequence of Unmodified BoNT/A)
MPFVNKQFNYKDPVNGVDIAYIKIPNAGQMQPVKAFKIHNKIWVIPERDTFTNPEEGDLNPPPEAKQVPVSYYDSTYLSTDNE
KDNYLKGVTKLFERIYSTDLGRMLLTSIVRGIPFWGGSTIDTELKVIDTNCINVIQPDGSYRSEELNLVIIGPSADIIQFECK
SFGHEVLNLTRNGYGSTQYIRFSPDFTFGFEESLEVDTNPLLGAGKFATDPAVTLAHELIHAGHRLYGIAINPNRVFKVNTNA
YYEMSGLEVSFEELRTFGGHDAKFIDSLQENEFRLYYYNKFKDIASTLNKAKSIVGTTASLQYMKNVFKEKYLLSEDTSGKFS
VDKLKFDKLYKMLTEIYTEDNFVKFFKVLNRKTYLNFDKAVFKINIVPKVNYTIYDGFNLRNTNLAANFNGQNTEINNMNFTK
LKNFTGLFEFYKLLCVRGIITSKTKSLDKGYNKALNDLCIKVNNWDLFFSPSEDNFTNDLNKGEEITSDTNIEAAEENISLDL
IQQYYLTFNFDNEPENISIENLSSDIIGQLELMPNIERFPNGKKYELDKYTMFHYLRAQEFEHGKSRIALTNSVNEALLNPSR
VYTFFSSDYVKKVNKATEAAMFLGWVEQLVYDFTDETSEVSTTDKIADITIIIPYIGPALNIGNMLYKDDFVGALIFSGAVIL
LEFIPEIAIPVLGTFALVSYIANKVLTVQTIDNALSKRNEKWDEVYKYIVTNWLAKVNTQIDLIRKKMKEALENQAEATKAII
NYQYNQYTEEEKNNINFNIDDLSSKLNESINKAMININKFLNQCSVSYLMNSMIPYGVKRLEDFDASLKDALLKYIYDNRGTL
IGQVDRLKDKVNNTLSTDIPFQLSKYVDNQRLLSTFTEYIKNIINTSILNLRYESNHLIDLSRYASKINIGSKVNFDPIDKNQ
IQLFNLESSKIEVILKNAIVYNSMYENFSTSFWIRIPKYFNSISLNNEYTIINCMENNSGWKVSLNYGEIIWTLQDTQEIKQR
VVFKYSQMINISDYINRWIFVTITNNRLNNSKIYINGRLIDQKPISNLGNIHASNNIMFKLDGCRDTHRYIWIKYFNLFDKEL
NEKEIKDLYDNQSNSGILKDFWGDYLQYDKPYYMLNLYDPNKYVDVNNVGIRGYMYLKGPRGSVMTTNIYLNSSLYRGTKFII
KKYASGNKDNIVRNNDRVYINVVVKNKEYRLATNASQAGVEKILSALEIPDVGNLSQVVVMKSKNDQGITNKCKMNLQDNNGN
DIGFIGFHQFNNIAKLVASNWYNRQIERSSRTLGCSWEFIPVDDGWGERPL
SEQ ID NO: 14 (Nucleotide Sequence of Modified BoNT/A “Cat-A”)
ATGCCATTCGTCAACAAGCAATTCAACTACAAAGACCCAGTCAACGGCGTCGACATCGCATACATCAAGATTCCGAACGCCGG
TCAAATGCAGCCGGTTAAGGCTTTTAAGATCCACAACAAGATTTGGGTTATCCCGGAGCGTGACACCTTCACGAACCCGGAAG
AAGGCGATCTGAACCCGCCACCGGAAGCGAAGCAAGTCCCTGTCAGCTACTACGATTCGACGTACCTGAGCACGGATAACGAA
AAAGATAACTACCTGAAAGGTGTGACCAAGCTGTTCGAACGTATCTACAGCACGGATCTGGGTCGCATGCTGCTGACTAGCAT
TGTTCGCGGTATCCCGTTCTGGGGTGGTAGCACGATTGACACCGAACTGAAGGTTATCGACACTAACTGCATTAACGTTATTC
AACCGGATGGTAGCTATCGTAGCGAAGAGCTGAATCTGGTCATCATTGGCCCGAGCGCAGACATTATCCAATTCGAGTGCAAG
AGCTTTGGTCACGAGGTTCTGAATCTGACCCGCAATGGCTATGGTAGCACCCAGTACATTCGTTTTTCGCCGGATTTTACCTT
CGGCTTTGAAGAGAGCCTGGAGGTTGATACCAATCCGTTGCTGGGTGCGGGCAAATTCGCTACCGATCCGGCTGTCACGCTGG
CCCATGAACTGATCCACGCAGGCCACCGCCTGTACGGCATTGCCATCAACCCAAACCGTGTGTTCAAGGTTAATACGAATGCA
TACTACGAGATGAGCGGCCTGGAAGTCAGCTTCGAAGAACTGCGCACCTTCGGTGGCCATGACGCTAAATTCATTGACAGCTT
GCAAGAGAATGAGTTCCGTCTGTACTACTATAACAAATTCAAAGACATTGCAAGCACGTTGAACAAGGCCAAAAGCATCGTTG
GTACTACCGCGTCGTTGCAGTATATGAAGAATGTGTTTAAAGAGAAGTACCTGCTGTCCGAGGATACCTCCGGCAAGTTTAGC
GTTGATAAGCTGAAGTTTGACAAACTGTACAAGATGCTGACCGAGATTTACACCGAGGACAACTTTGTGAAATTCTTCAAAGT
GTTGAATCGTAAAACCTATCTGAATTTTGACAAAGCGGTTTTCAAGATTAACATCGTGCCGAAGGTGAACTACACCATCTATG
ACGGTTTTAACCTGCGTAACACCAACCTGGCGGCGAACTTTAACGGTCAGAATACGGAAATCAACAACATGAATTTCACGAAG
TTGAAGAACTTCACGGGTCTGTTCGAGTTCTATAAGCTGCTGTGCGTGCGCGGTATCATCACCAGCAAAACCAAAAGCCTGGA
CAAAGGCTACAACAAGGCGCTGAATGACCTGTGCATTAAGGTAAACAATTGGGATCTGTTCTTTTCGCCATCCGAAGATAATT
TTACCAACGACCTGAACAAGGGTGAAGAAATCACCAGCGATACGAATATTGAAGCAGCGGAAGAGAATATCAGCCTGGATCTG
ATCCAGCAGTACTATCTGACCTTTAACTTCGACAATGAACCGGAGAACATTAGCATTGAGAATCTGAGCAGCGACATTATCGG
TCAGCTGGAACTGATGCCGAATATCGAACGTTTCCCGAACGGCAAAAAGTACGAGCTGGACAAGTACACTATGTTCCATTACC
TGCGTGCACAGGAGTTTGAACACGGTAAAAGCCGTATCGCGCTGACCAACAGCGTTAACGAGGCCCTGCTGAACCCGAGCCGT
GTCTATACCTTCTTCAGCAGCGACTATGTTAAGAAAGTGAACAAAGCCACTGAGGCCGCGATGTTCCTGGGCTGGGTGGAACA
GCTGGTATATGACTTCACGGACGAGACGAGCGAAGTGAGCACTACCGACAAAATTGCTGATATTACCATCATTATCCCGTATA
TTGGTCCGGCACTGAACATTGGCAACATGCTGTACAAAGACGATTTTGTGGGTGCCCTGATCTTCTCCGGTGCCGTGATTCTG
CTGGAGTTCATTCCGGAGATTGCGATCCCGGTGTTGGGTACCTTCGCGCTGGTGTCCTACATCGCGAATAAGGTTCTGACGGT
TCAGACCATCGATAACGCGCTGTCGAAACGTAATGAAAAATGGGACGAGGTTTACAAATACATTGTTACGAATTGGCTGGCGA
AAGTCAATACCCAGATCGACCTGATCCGTAAGAAAATGAAAGAGGCGCTGGAGAATCAGGCGGAGGCCACCAAAGCAATTATC
AACTACCAATACAACCAGTACACGGAAGAAGAGAAGAATAACATTAACTTCAATATCGATGATTTGAGCAGCAAGCTGAATGA
ATCTATCAACAAAGCGATGATCAATATCAACAAGTTTTTGAATCAGTGTAGCGTTTCGTACCTGATGAATAGCATGATTCCGT
ATGGCGTCAAACGTCTGGAGGACTTCGACGCCAGCCTGAAAGATGCGTTGCTGAAATACATTTACGACAATCGTGGTACGCTG
ATTGGCCAAGTTGACCGCTTGAAAGACAAAGTTAACAATACCCTGAGCACCGACATCCCATTTCAACTGAGCAAGTATGTTGA
TAATCAACGTCTGTTGAGCACTTTCACCGAGTATATCAAAAACATCATCAATACTAGCATTCTGAACCTGCGTTACGAGAGCA
AGCATCTGATTGATCTGAGCCGTTATGCTAGCAAGATCAACATCGGTAGCAAGGTCAATTTTGACCCGATCGATAAGAACCAG
ATCCAGCTGTTTAATCTGGAATCGAGCAAAATTGAGGTTATCCTGAAAAAGGCCATTGTCTACAACTCCATGTACGAGAATTT
CTCCACCAGCTTCTGGATTCGCATCCCGAAATACTTCAACAAGATTAGCCTGAACAACGAGTATACTATCATCAACTGTATGG
AGAACAACAGCGGTTGGAAGGTGTCTCTGAACTATGGTGAGATCATTTGGACCTTGCAGGACACCAAAGAGATCAAGCAGCGC
GTCGTGTTCAAGTACTCTCAAATGATCAACATTTCCGATTACATTAATCGTTGGATCTTCGTGACCATTACGAATAACCGTCT
GAATAAGAGCAAGATTTACATCAATGGTCGCTTGATCGATCAGAAACCGATTAGCAACCTGGGTAATATCCACGCAAGCAACA
AGATTATGTTCAAATTGGACGGTTGCCGCGATACCCATCGTTATATCTGGATCAAGTATTTCAACCTGTTTGATAAAGAACTG
AATGAGAAGGAGATCAAAGATTTGTATGACAACCAATCTAACAGCGGCATTTTGAAGGACTTCTGGGGCGATTATCTGCAATA
CGATAAGCCGTACTATATGCTGAACCTGTATGATCCGAACAAATATGTGGATGTCAATAATGTGGGTATTCGTGGTTACATGT
ATTTGAAGGGTCCGCGTGGCAGCGTTATGACGACCAACATTTACCTGAACTCTAGCCTGTACCGTGGTACGAAATTCATCATT
AAGAAATATGCCAGCGGCAACAAAGATAACATTGTGCGTAATAACGATCGTGTCTACATCAACGTGGTCGTGAAGAATAAAGA
GTACCGTCTGGCGACCAACGCTTCGCAGGCGGGTGTTGAGAAAATTCTGAGCGCGTTGGAGATCCCTGATGTCGGTAATCTGA
GCCAAGTCGTGGTTATGAAGAGCAAGAACGACAAGGGTATCACTAACAAGTGCAAGATGAACCTGCAAGACAACAATGGTAAC
GACATCGGCTTTATTGGTTTCCACCAGTTCAACAATATTGCTAAACTGGTAGCGAGCAATTGGTACAATCGTCAGATTGAGCG
CAGCAGCcGTACTTTGGGCTGTAGCTGGGAGTTTATCCCGGTCGATGATGGTTGGGGCGAACGTCCGCTG
SEQ ID NO: 15 (Polypeptide Sequence of Modified BoNT/A ″Cat-A″)
MPFVNKQFNYKDPVNGVDIAYIKIPNAGQMQPVKAFKIHNKIWVIPERDTFTNPEEGDLNPPPEAKQVPVSYYDSTYLSTDNE
KDNYLKGVTKLFERIYSTDLGRMLLTSIVRGIPFWGGSTIDTELKVIDTNCINVIQPDGSYRSEELNLVIIGPSADIIQFECK
SFGHEVLNLTRNGYGSTQYIRFSPDFTFGFEESLEVDTNPLLGAGKFATDPAVTLAHELIHAGHRLYGIAINPNRVFKVNTNA
YYEMSGLEVSFEELRTFGGHDAKFIDSLQENEFRLYYYNKFKDIASTLNKAKSIVGTTASLQYMKNVFKEKYLLSEDTSGKFS
VDKLKFDKLYKMLTEIYTEDNFVKFFKVLNRKTYLNFDKAVFKINIVPKVNYTIYDGFNLRNTNLAANFNGQNTEINNMNFTK
LKNFTGLFEFYKLLCVRGIITSKTKSLDKGYNKALNDLCIKVNNWDLFFSPSEDNFTNDLNKGEEITSDTNIEAAEENISLDL
IQQYYLTFNFDNEPENISIENLSSDIIGQLELMPNIERFPNGKKYELDKYTMFHYLRAQEFEHGKSRIALTNSVNEALLNPSR
VYTFFSSDYVKKVNKATEAAMFLGWVEQLVYDFTDETSEVSTTDKIADITIIIPYIGPALNIGNMLYKDDFVGALIFSGAVIL
LEFIPEIAIPVLGTFALVSYIANKVLTVQTIDNALSKRNEKWDEVYKYIVTNWLAKVNTQIDLIRKKMKEALENQAEATKAII
NYQYNQYTEEEKNNINFNIDDLSSKLNESINKAMININKFLNQCSVSYLMNSMIPYGVKRLEDFDASLKDALLKYIYDNRGTL
IGQVDRLKDKVNNTLSTDIPFQLSKYVDNQRLLSTFTEYIKNIINTSILNLRYESKHLIDLSRYASKINIGSKVNFDPIDKNQ
IQLFNLESSKIEVILKKAIVYNSMYENFSTSFWIRIPKYFNKISLNNEYTIINCMENNSGWKVSLNYGEIIWTLQDTKEIKQR
VVFKYSQMINISDYINRWIFVTITNNRLNKSKIYINGRLIDQKPISNLGNIHASNKIMFKLDGCRDTHRYIWIKYFNLFDKEL
NEKEIKDLYDNQSNSGILKDFWGDYLQYDKPYYMLNLYDPNKYVDVNNVGIRGYMYLKGPRGSVMTTNIYLNSSLYRGTKFII
KKYASGNKDNIVRNNDRVYINVVVKNKEYRLATNASQAGVEKILSALEIPDVGNLSQVVVMKSKNDKGITNKCKMNLQDNNGN
DIGFIGFHQFNNIAKLVASNWYNRQIERSSRTLGCSWEFIPVDDGWGERPL
SEQ ID NO: 16 (Nucleotide Sequence of Modified BoNT/A “Cat-B”)
ATGCCATTCGTCAACAAGCAATTCAACTACAAAGACCCAGTCAACGGCGTCGACATCGCATACATCAAGATTCCGAACGCCGG
TCAAATGCAGCCGGTTAAGGCTTTTAAGATCCACAACAAGATTTGGGTTATCCCGGAGCGTGACACCTTCACGAACCCGGAAG
AAGGCGATCTGAACCCGCCACCGGAAGCGAAGCAAGTCCCTGTCAGCTACTACGATTCGACGTACCTGAGCACGGATAACGAA
AAAGATAACTACCTGAAAGGTGTGACCAAGCTGTTCGAACGTATCTACAGCACGGATCTGGGTCGCATGCTGCTGACTAGCAT
TGTTCGCGGTATCCCGTTCTGGGGTGGTAGCACGATTGACACCGAACTGAAGGTTATCGACACTAACTGCATTAACGTTATTC
AACCGGATGGTAGCTATCGTAGCGAAGAGCTGAATCTGGTCATCATTGGCCCGAGCGCAGACATTATCCAATTCGAGTGCAAG
AGCTTTGGTCACGAGGTTCTGAATCTGACCCGCAATGGCTATGGTAGCACCCAGTACATTCGTTTTTCGCCGGATTTTACCTT
CGGCTTTGAAGAGAGCCTGGAGGTTGATACCAATCCGTTGCTGGGTGCGGGCAAATTCGCTACCGATCCGGCTGTCACGCTGG
CCCATGAACTGATCCACGCAGGCCACCGCCTGTACGGCATTGCCATCAACCCAAACCGTGTGTTCAAGGTTAATACGAATGCA
TACTACGAGATGAGCGGCCTGGAAGTCAGCTTCGAAGAACTGCGCACCTTCGGTGGCCATGACGCTAAATTCATTGACAGCTT
GCAAGAGAATGAGTTCCGTCTGTACTACTATAACAAATTCAAAGACATTGCAAGCACGTTGAACAAGGCCAAAAGCATCGTTG
GTACTACCGCGTCGTTGCAGTATATGAAGAATGTGTTTAAAGAGAAGTACCTGCTGTCCGAGGATACCTCCGGCAAGTTTAGC
GTTGATAAGCTGAAGTTTGACAAACTGTACaAGATGCTGACCGAGATTTACACCGAGGACAACTTTGTGAAATTCTTCAAAGT
GTTGAATCGTAAAACCTATCTGAATTTTGACAAAGCGGTTTTCAAGATTAACATCGTGCCGAAGGTGAACTACACCATCTATG
ACGGTTTTAACCTGCGTAACACCAACCTGGCGGCGAACTTTAACGGTCAGAATACGGAAATCAACAACATGAATTTCACGAAG
TTGAAGAACTTCACGGGTCTGTTCGAGTTCTATAAGCTGCTGTGCGTGCGCGGTATCATCACCAGCAAAACCAAAAGCCTGGA
CAAAGGCTACAACAAGGCGCTGAATGACCTGTGCATTAAGGTAAACAATTGGGATCTGTTCTTTTCGCCATCCGAAGATAATT
TTACCAACGACCTGAACAAGGGTGAAGAAATCACCAGCGATACGAATATTGAAGCAGCGGAAGAGAATATCAGCCTGGATCTG
ATCCAGCAGTACTATCTGACCTTTAACTTCGACAATGAACCGGAGAACATTAGCATTGAGAATCTGAGCAGCGACATTATCGG
TCAGCTGGAACTGATGCCGAATATCGAACGTTTCCCGAACGGCAAAAAGTACGAGCTGGACAAGTACACTATGTTCCATTACC
TGCGTGCACAGGAGTTTGAACACGGTAAAAGCCGTATCGCGCTGACCAACAGCGTTAACGAGGCCCTGCTGAACCCGAGCCGT
GTCTATACCTTCTTCAGCAGCGACTATGTTAAGAAAGTGAACAAAGCCACTGAGGCCGCGATGTTCCTGGGCTGGGTGGAACA
GCTGGTATATGACTTCACGGACGAGACGAGCGAAGTGAGCACTACCGACAAAATTGCTGATATTACCATCATTATCCCGTATA
TTGGTCCGGCACTGAACATTGGCAACATGCTGTACAAAGACGATTTTGTGGGTGCCCTGATCTTCTCCGGTGCCGTGATTCTG
CTGGAGTTCATTCCGGAGATTGCGATCCCGGTGTTGGGTACCTTCGCGCTGGTGTCCTACATCGCGAATAAGGTTCTGACGGT
TCAGACCATCGATAACGCGCTGTCGAAACGTAATGAAAAATGGGACGAGGTTTACAAATACATTGTTACGAATTGGCTGGCGA
AAGTCAATACCCAGATCGACCTGATCCGTAAGAAAATGAAAGAGGCGCTGGAGAATCAGGCGGAGGCCACCAAAGCAATTATC
AACTACCAATACAACCAGTACACGGAAGAAGAGAAGAATAACATTAACTTCAATATCGATGATTTGAGCAGCAAGCTGAATGA
ATCTATCAACAAAGCGATGATCAATATCAACAAGTTTTTGAATCAGTGTAGCGTTTCGTACCTGATGAATAGCATGATTCCGT
ATGGCGTCAAACGTCTGGAGGACTTCGACGCCAGCCTGAAAGATGCGTTGCTGAAATACATTTACGACAaTCGTGGTACGCTG
ATTGGCCAAGTTGACCGCTTGAAAGACAAAGTTAACAATACCCTGAGCACCGACATCCCATTTCAACTGAGCAAGTATGTTGA
TAATCAACGTCTGTTGAGCACTTTCACCGAGTATATCAAAAACATCATCAATACTAGCATTCTGAACCTGCGTTACGAGAGCA
ATCATCTGATTGATCTGAGCCGTTATGCTAGCAAGATCAACATCGGTAGCAAGGTCAATTTTGACCCGATCGATAAGAACCAG
ATCCAGCTGTTTAATCTGGAATCGAGCAAAATTGAGGTTATCCTGAAAAAGGCCATTGTCTACAACTCCATGTACGAGAATTT
CTCCACCAGCTTCTGGATTCGCATCCCGAAATACTTCAAGAAGATTAGCCTGAACAACGAGTATACTATCATCAACTGTATGG
AGAACAACAGCGGTTGGAAGGTGTCTCTGAACTATGGTGAGATCATTTGGACCTTGCAGGACACCAAAGAGATCAAGCAGCGC
GTCGTGTTCAAGTACTCTCAAATGATCAACATTTCCGATTACATTAATCGTTGGATCTTCGTGACCATTACGAATAACCGTCT
GAATAAGAGCAAGATTTACATCAATGGTCGCTTGATCGATCAGAAACCGATTAGCAACCTGGGTAATATCCACGCAAGCAACA
AGATTATGTTCAAATTGGACGGTTGCCGCGATACCCATCGTTATATCTGGATCAAGTATTTCAACCTGTTTGATAAAGAACTG
AATGAGAAGGAGATCAAAGATTTGTATGACAACCAATCTAACAGCGGCATTTTGAAGGACTTCTGGGGCGATTATCTGCAATA
CGATAAGCCGTACTATATGCTGAACCTGTATGATCCGAACAAATATGTGGATGTCAATAATGTGGGTATTCGTGGTTACATGT
ATTTGAAGGGTCCGCGTGGCAGCGTTATGACGACCAACATTTACCTGAACTCTAGCCTGTACCGTGGTACGAAATTCATCATT
AAGAAATATGCCAGCGGCAACAAAGATAACATTGTGCGTAATAACGATCGTGTCTACATCAACGTGGTCGTGAAGAATAAAGA
GTACCGTCTGGCGACCAACGCTTCGCAGGCGGGTGTTGAGAAAATTCTGAGCGCGTTGGAGATCCCTGATGTCGGTAATCTGA
GCCAAGTCGTGGTTATGAAGAGCAAGAACGACAAGGGTATCACTAACAAGTGCAAGATGAACCTGCAAGACAACAATGGTAAC
GACATCGGCTTTATTGGTTTCCACCAGTTCAACAATATTGCTAAACTGGTAGCGAGCAATTGGTACAATCGTCAGATTGAGCG
CAGCAGCCGTACTTTGGGCTGTAGCTGGGAGTTTATCCCGGTCGATGATGGTTGGGGCGAACGTCCGCTG
SEQ ID NO: 17 (Polypeptide Sequence of Modified BoNT/A ″Cat-B″)
MPFVNKQFNYKDPVNGVDIAYIKIPNAGQMQPVKAFKIHNKIWVIPERDTFTNPEEGDLNPPPEAKQVPVSYYDSTYLSTDNE
KDNYLKGVTKLFERIYSTDLGRMLLTSIVRGIPFWGGSTIDTELKVIDTNCINVIQPDGSYRSEELNLVIIGPSADIIQFECK
SFGHEVLNLTRNGYGSTQYIRFSPDFTFGFEESLEVDTNPLLGAGKFATDPAVTLAHELIHAGHRLYGIAINPNRVFKVNTNA
YYEMSGLEVSFEELRTFGGHDAKFIDSLQENEFRLYYYNKFKDIASTLNKAKSIVGTTASLQYMKNVFKEKYLLSEDTSGKFS
VDKLKFDKLYKMLTEIYTEDNFVKFFKVLNRKTYLNFDKAVFKINIVPKVNYTIYDGFNLRNTNLAANFNGQNTEINNMNFTK
LKNFTGLFEFYKLLCVRGIITSKTKSLDKGYNKALNDLCIKVNNWDLFFSPSEDNFTNDLNKGEEITSDTNIEAAEENISLDL
IQQYYLTFNFDNEPENISIENLSSDIIGQLELMPNIERFPNGKKYELDKYTMFHYLRAQEFEHGKSRIALTNSVNEALLNPSR
VYTFFSSDYVKKVNKATEAAMFLGWVEQLVYDFTDETSEVSTTDKIADITIIIPYIGPALNIGNMLYKDDFVGALIFSGAVIL
LEFIPEIAIPVLGTFALVSYIANKVLTVQTIDNALSKRNEKWDEVYKYIVTNWLAKVNTQIDLIRKKMKEALENQAEATKAII
NYQYNQYTEEEKNNINFNIDDLSSKLNESINKAMININKFLNQCSVSYLMNSMIPYGVKRLEDFDASLKDALLKYIYDNRGTL
IGQVDRLKDKVNNTLSTDIPFQLSKYVDNQRLLSTFTEYIKNIINTSILNLRYESNHLIDLSRYASKINIGSKVNFDPIDKNQ
IQLFNLESSKIEVILKKAIVYNSMYENFSTSFWIRIPKYFKKISLNNEYTIINCMENNSGWKVSLNYGEIIWTLQDTKEIKQR
VVFKYSQMINISDYINRWIFVTITNNRLNKSKIYINGRLIDQKPISNLGNIHASNKIMFKLDGCRDTHRYIWIKYFNLFDKEL
NEKEIKDLYDNQSNSGILKDFWGDYLQYDKPYYMLNLYDPNKYVDVNNVGIRGYMYLKGPRGSVMTTNIYLNSSLYRGTKFII
KKYASGNKDNIVRNNDRVYINVVVKNKEYRLATNASQAGVEKILSALEIPDVGNLSQVVVMKSKNDKGITNKCKMNLQDNNGN
DIGFIGFHQFNNIAKLVASNWYNRQIERSSRTLGCSWEFIPVDDGWGERPL
SEQ ID NO: 18 (Nucleotide Sequence of Modified BoNT/A “Cat-C”)
ATGCCATTCGTCAACAAGCAATTCAACTACAAAGACCCAGTCAACGGCGTCGACATCGCATACATCAAGATTCCGAACGCCGG
TCAAATGCAGCCGGTTAAGGCTTTTAAGATCCACAACAAGATTTGGGTTATCCCGGAGCGTGACACCTTCACGAACCCGGAAG
AAGGCGATCTGAACCCGCCACCGGAAGCGAAGCAAGTCCCTGTCAGCTACTACGATTCGACGTACCTGAGCACGGATAACGAA
AAAGATAACTACCTGAAAGGTGTGACCAAGCTGTTCGAACGTATCTACAGCACGGATCTGGGTCGCATGCTGCTGACTAGCAT
TGTTCGCGGTATCCCGTTCTGGGGTGGTAGCACGATTGACACCGAACTGAAGGTTATCGACACTAACTGCATTAACGTTATTC
AACCGGATGGTAGCTATCGTAGCGAAGAGCTGAATCTGGTCATCATTGGCCCGAGCGCAGACATTATCCAATTCGAGTGCAAG
AGCTTTGGTCACGAGGTTCTGAATCTGACCCGCAATGGCTATGGTAGCACCCAGTACATTCGTTTTTCGCCGGATTTTACCTT
CGGCTTTGAAGAGAGCCTGGAGGTTGATACCAATCCGTTGCTGGGTGCGGGCAAATTCGCTACCGATCCGGCTGTCACGCTGG
CCCATGAACTGATCCACGCAGGCCACCGCCTGTACGGCATTGCCATCAACCCAAACCGTGTGTTCAAGGTTAATACGAATGCA
TACTACGAGATGAGCGGCCTGGAAGTCAGCTTCGAAGAACTGCGCACCTTCGGTGGCCATGACGCTAAATTCATTGACAGCTT
GCAAGAGAATGAGTTCCGTCTGTACTACTATAACAAATTCAAAGACATTGCAAGCACGTTGAACAAGGCCAAAAGCATCGTTG
GTACTACCGCGTCGTTGCAGTATATGAAGAATGTGTTTAAAGAGAAGTACCTGCTGTCCGAGGATACCTCCGGCAAGTTTAGC
GTTGATAAGCTGAAGTTTGACAAACTGTACAAGATGCTGACCGAGATTTACACCGAGGACAACTTTGTGAAATTCTTCAAAGT
GTTGAATCGTAAAACCTATCTGAATTTTGACAAAGCGGTTTTCAAGATTAACATCGTGCCGAAGGTGAACTACACCATCTATG
ACGGTTTTAACCTGCGTAACACCAACCTGGCGGCGAACTTTAACGGTCAGAATACGGAAATCAACAACATGAATTTCACGAAG
TTGAAGAACTTCACGGGTCTGTTCGAGTTCTATAAGCTGCTGTGCGTGCGCGGTATCATCACCAGCAAAACCAAAAGCCTGGA
CAAAGGCTACAACAAGGCGCTGAATGACCTGTGCATTAAGGTAAACAATTGGGATCTGTTCTTTTCGCCATCCGAAGATAATT
TTACCAACGACCTGAACAAGGGTGAAGAAATCACCAGCGATACGAATATTGAAGCAGCGGAAGAGAATATCAGCCTGGATCTG
ATCCAGCAGTACTATCTGACCTTTAACTTCGACAATGAACCGGAGAACATTAGCATTGAGAATCTGAGCAGCGACATTATCGG
TCAGCTGGAACTGATGCCGAATATCGAACGTTTCCCGAACGGCAAAAAGTACGAGCTGGACAAGTACACTATGTTCCATTACC
TGCGTGCACAGGAGTTTGAACACGGTAAAAGCCGTATCGCGCTGACCAACAGCGTTAACGAGGCCCTGCTGAACCCGAGCCGT
GTCTATACCTTCTTCAGCAGCGACTATGTTAAGAAAGTGAACAAAGCCACTGAGGCCGCGATGTTCCTGGGCTGGGTGGAACA
GCTGGTATATGACTTCACGGACGAGACGAGCGAAGTGAGCACTACCGACAAAATTGCTGATATTACCATCATTATCCCGTATA
TTGGTCCGGCACTGAACATTGGCAACATGCTGTACAAAGACGATTTTGTGGGTGCCCTGATCTTCTCCGGTGCCGTGATTCTG
CTGGAGTTCATTCCGGAGATTGCGATCCCGGTGTTGGGTACCTTCGCGCTGGTGTCCTACATCGCGAATAAGGTTCTGACGGT
TCAGACCATCGATAACGCGCTGTCGAAACGTAATGAAAAATGGGACGAGGTTTACAAATACATTGTTACGAATTGGCTGGCGA
AAGTCAATACCCAGATCGACCTGATCCGTAAGAAAATGAAAGAGGCGCTGGAGAATCAGGCGGAGGCCACCAAAGCAATTATC
AACTACCAATACAACCAGTACACGGAAGAAGAGAAGAATAACATTAACTTCAATATCGATGATTTGAGCAGCAAGCTGAATGA
ATCTATCAACAAAGCGATGATCAATATCAACAAGTTTTTGAATCAGTGTAGCGTTTCGTACCTGATGAATAGCATGATTCCGT
ATGGCGTCAAACGTCTGGAGGACTTCGACGCCAGCCTGAAAGATGCGTTGCTGAAATACATTTACGACAATCGTGGTACGCTG
ATTGGCCAAGTTGACCGCTTGAAAGACAAAGTTAACAATACCCTGAGCACCGACATCCCATTTCAACTGAGCAAGTATGTTGA
TAATCAACGTCTGTTGAGCACTTTCACCGAGTATATCAAAAACATCATCAATACTAGCATTCTGAACCTGCGTTACGAGAGCA
ATCATCTGATTGATCTGAGCCGTTATGCTAGCAAGATCAACATCGGTAGCAAGGTCAATTTTGACCCGATCGATAAGAACCAG
ATCCAGCTGTTTAATCTGGAATCGAGCAAAATTGAGGTTATCCTGAAAAAGGCCATTGTCTACAACTCCATGTACGAGAATTT
CTCCACCAGCTTCTGGATTCGCATCCCGAAATACTTCAACAAGATTAGCCTGAACAACGAGTATACTATCATCAACTGTATGG
AGAACAACAGCGGTTGGAAGGTGTCTCTGAACTATGGTGAGATCATTTGGACCTTGCAGGACACCAAAGAGATCAAGCAGCGC
GTCGTGTTCAAGTACTCTCAAATGATCAACATTTCCGATTACATTAATCGTTGGATCTTCGTGACCATTACGAATAACCGTCT
GAAGAAGAGCAAGATTTACATCAATGGTCGCTTGATCGATCAGAAACCGATTAGCAACCTGGGTAATATCCACGCAAGCAACA
AGATTATGTTCAAATTGGACGGTTGCCGCGATACCCATCGTTATATCTGGATCAAGTATTTCAACCTGTTTGATAAAGAACTG
AATGAGAAGGAGATCAAAGATTTGTATGACAACCAATCTAACAGCGGCATTTTGAAGGACTTCTGGGGCGATTATCTGCAATA
CGATAAGCCGTACTATATGCTGAACCTGTATGATCCGAACAAATATGTGGATGTCAATAATGTGGGTATTCGTGGTTACATGT
ATTTGAAGGGTCCGCGTGGCAGCGTTATGACGACCAACATTTACCTGAACTCTAGCCTGTACCGTGGTACGAAATTCATCATT
AAGAAATATGCCAGCGGCAACAAAGATAACATTGTGCGTAATAACGATCGTGTCTACATCAACGTGGTCGTGAAGAATAAAGA
GTACCGTCTGGCGACCAACGCTTCGCAGGCGGGTGTTGAGAAAATTCTGAGCGCGTTGGAGATCCCTGATGTCGGTAATCTGA
GCCAAGTCGTGGTTATGAAGAGCAAGAACGACAAGGGTATCACTAACAAGTGCAAGATGAACCTGCAAGACAACAATGGTAAC
GACATCGGCTTTATTGGTTTCCACCAGTTCAACAATATTGCTAAACTGGTAGCGAGCAATTGGTACAATCGTCAGATTGAGCG
CAGCAGCCGTACTTTGGGCTGTAGCTGGGAGTTTATCCCGGTCGATGATGGTTGGGGCGAACGTCCGCTG
SEQ ID NO: 19 (Polypeptide Sequence of Modified BoNT/A ″Cat-C″)
MPFVNKQFNYKDPVNGVDIAYIKIPNAGQMQPVKAFKIHNKIWVIPERDTFTNPEEGDLNPPPEAKQVPVSYYDSTYLSTDNE
KDNYLKGVTKLFERIYSTDLGRMLLTSIVRGIPFWGGSTIDTELKVIDTNCINVIQPDGSYRSEELNLVIIGPSADIIQFECK
SFGHEVLNLTRNGYGSTQYIRFSPDFTFGFEESLEVDTNPLLGAGKFATDPAVTLAHELIHAGHRLYGIAINPNRVFKVNTNA
YYEMSGLEVSFEELRTFGGHDAKFIDSLQENEFRLYYYNKFKDIASTLNKAKSIVGTTASLQYMKNVFKEKYLLSEDTSGKFS
VDKLKFDKLYKMLTEIYTEDNFVKFFKVLNRKTYLNFDKAVFKINIVPKVNYTIYDGFNLRNTNLAANFNGQNTEINNMNFTK
LKNFTGLFEFYKLLCVRGIITSKTKSLDKGYNKALNDLCIKVNNWDLFFSPSEDNFTNDLNKGEEITSDTNIEAAEENISLDL
IQQYYLTFNFDNEPENISIENLSSDIIGQLELMPNIERFPNGKKYELDKYTMFHYLRAQEFEHGKSRIALTNSVNEALLNPSR
VYTFFSSDYVKKVNKATEAAMFLGWVEQLVYDFTDETSEVSTTDKIADITIIIPYIGPALNIGNMLYKDDFVGALIFSGAVIL
LEFIPEIAIPVLGTFALVSYIANKVLTVQTIDNALSKRNEKWDEVYKYIVTNWLAKVNTQIDLIRKKMKEALENQAEATKAII
NYQYNQYTEEEKNNINFNIDDLSSKLNESINKAMININKFLNQCSVSYLMNSMIPYGVKRLEDFDASLKDALLKYIYDNRGTL
IGQVDRLKDKVNNTLSTDIPFQLSKYVDNQRLLSTFTEYIKNIINTSILNLRYESNHLIDLSRYASKINIGSKVNFDPIDKNQ
IQLFNLESSKIEVILKKAIVYNSMYENFSTSFWIRIPKYFNKISLNNEYTIINCMENNSGWKVSLNYGEIIWTLQDTKEIKQR
VVFKYSQMINISDYINRWIFVTITNNRLKKSKIYINGRLIDQKPISNLGNIHASNKIMFKLDGCRDTHRYIWIKYFNLFDKEL
NEKEIKDLYDNQSNSGILKDFWGDYLQYDKPYYMLNLYDPNKYVDVNNVGIRGYMYLKGPRGSVMTTNIYLNSSLYRGTKFII
KKYASGNKDNIVRNNDRVYINVVVKNKEYRLATNASQAGVEKILSALEIPDVGNLSQVVVMKSKNDKGITNKCKMNLQDNNGN
DIGFIGFHQFNNIAKLVASNWYNRQIERSSRTLGCSWEFIPVDDGWGERPL
SEQ ID NO: 20 (Nucleotide Sequence of Modified BoNT/A “Cat-D”)
ATGCCATTCGTCAACAAGCAATTCAACTACAAAGACCCAGTCAACGGCGTCGACATCGCATACATCAAGATTCCGAACGCCGG
TCAAATGCAGCCGGTTAAGGCTTTTAAGATCCACAACAAGATTTGGGTTATCCCGGAGCGTGACACCTTCACGAACCCGGAAG
AAGGCGATCTGAACCCGCCACCGGAAGCGAAGCAAGTCCCTGTCAGCTACTACGATTCGACGTACCTGAGCACGGATAACGAA
AAAGATAACTACCTGAAAGGTGTGACCAAGCTGTTCGAACGTATCTACAGCACGGATCTGGGTCGCATGCTGCTGACTAGCAT
TGTTCGCGGTATCCCGTTCTGGGGTGGTAGCACGATTGACACCGAACTGAAGGTTATCGACACTAACTGCATTAACGTTATTC
AACCGGATGGTAGCTATCGTAGCGAAGAGCTGAATCTGGTCATCATTGGCCCGAGCGCAGACATTATCCAATTCGAGTGCAAG
AGCTTTGGTCACGAGGTTCTGAATCTGACCCGCAATGGCTATGGTAGCACCCAGTACATTCGTTTTTCGCCGGATTTTACCTT
CGGCTTTGAAGAGAGCCTGGAGGTTGATACCAATCCGTTGCTGGGTGCGGGCAAATTCGCTACCGATCCGGCTGTCACGCTGG
CCCATGAACTGATCCACGCAGGCCACCGCCTGTACGGCATTGCCATCAACCCAAACCGTGTGTTCAAGGTTAATACGAATGCA
TACTACGAGATGAGCGGCCTgGAAGTCAGCTTCGAAGAACTGCGCACCTTCGGTGGCCATGACGCTAAATTCATTGACAGCTT
GCAAGAGAATGAGTTCCGTCTGTACTACTATAACAAATTCAAAGACATTGCAAGCACGTTGAACAAGGCCAAAAGCATCGTTG
GTACTACCGCGTCGTTGCAGTATATGAAGAATGTGTTTAAAGAGAAGTACCTGCTGTCCGAGGATACCTCCGGCAAGTTTAGC
GTTGATAAGCTGAAGTTTGACAAACTGTACAAGATGCTGACCGAGATTTACACCGAGGACAACTTTGTGAAATTCTTCAAaGT
GTTGAATCGTAAAACCTATCTGAATTTTGACAAAGCGGTTTTCaAGATTAACATCGTGCCGAAGGTGAACTACACCATCTATG
ACGGTTTTAACCTGCGTAACACCAACCTGGCGGCGAACTTTAACGGTCAGAATACGGAAATCAACAACATGAATTTCACGAAG
TTGAAGAACTTCACGGGTCTGTTCGAGTTCTATAAGCTGCTGTGCGTGCGCGGTATCATCACCAGCAAAACCAAAAGCCTGGA
CAAAGGCTACAACAAGGCGCTGAATGACCTGTGCATTAAGGTAAACAATTGGGATCTGTTCTTTTCGCCATCCGAAGATAATT
TTACCAACGACCTGAACAAGGGTGAAGAAATCACCAGCGATACGAATATTGAAGCAGCGGAAGAGAATATCAGCCTGGATCTG
ATCCAGCAGTACTATCTGACCTTTAACTTCGACAATGAACCGGAGAACATTAGCATTGAGAATCTGAGCAGCGACATTATCGG
TCAGCTGGAACTGATGCCGAATATCGAACGTTTCCCGAACGGCAAAAAGTACGAGCTGGACAAGTACACTATGTTCCATTACC
TGCGTGCACAGGAGTTTGAACACGGTAAAAGCCGTATCGCGCTGACCAACAGCGTTAACGAGGCCCTGCTGAACCCGAGCCGT
GTCTATACCTTCTTCAGCAGCGACTATGTTAAGAAAGTGAACAAAGCCACTGAGGCCGCGATGTTCCTGGGCTGGGTGGAACA
GCTGGTATATGACTTCACGGACGAGACGAGCGAAGTGAGCACTACCGACAAAaTTGCTGATaTTACCATCATTATCCCGTATA
TTGGTCCGGCACTGAACATTGGCAACATGCTGTACAAAGACGATTTTGTGGGTGCCCTGATCTTCTCCGGTGCCGTGATTCTG
CTGGAGTTCATTCCGGAGATTGCGATCCCGGTGTTGGGTACCTTCGCGCTGGTGTCCTACATCGCGAATAAGGTTCTGACGGT
TCAGACCATCGATAACGCGCTGTCGAAACGTAATGAAAAATGGGACGAGGTTTACAAATACATTGTTACGAATTGGCTGGCGA
AAGTCaATACCCAGATCGACCTGATCCGTAAGAAAATGAAAGAGGCGCTGGAGAATCAGGCGGAGGCCACCAAAGCAATTATC
AACTACCAATACAACCAGTACACGGAAGAAGAGAAGAATAACATTAACTTCAATATCGATGATTTGAGCAGCAAGCTGAATGA
ATCTATCAACAAAGCGATGATCAATATCAACAAGTTTTTGAATCAGTGTAGCGTTTCGTACCTGATGAATAGCATGATTCCGT
ATGGCGTCAAACGTCTGGAGGACTTCGACGCCAGCCTGAAAGATGCGTTGCTGAAATACATTTACGACAATCGTGGTACGCTG
ATTGGCCAAGTTGACCGCTTGAAAGACAAAGTTAACAATACCCTGAGCACCGACATCCCATTTCAACTGAGCAAGTATGTTGA
TAATCAACGTCTGTTGAGCACTTTCACCGAGTATATCAAAAACATCATCAATACTAGCATTCTGAACCTGCGTTACGAGAGCA
ATCATCTGATtGATCTGAGCCGTTATGCAAGCAAGATCAACATCGGTAGCAAGGTCAATTTTGACCCGATCGATAAGAACCAG
ATCCAGCTGTTTAATCTGGAATCGAGCAAAATTGAGGTTATCCTGAAAAACGCCATTGTCTACAACTCCATGTACGAGAATTT
CTCCACCAGCTTCTGGATTCGCATCCCGAAATACTTCAACAGCATTAGCCTGAACAACGAGTATACTATCATCAACTGTATGG
AGAACAACAGCGGTTGGAAGGTGTCTCTGAACTATGGTGAGATCATTTGGACCTTGCAGGACACCCAAGAGATCAAGCAGCGC
GTCGTGTTCAAGTACTCTCAAATGATCAACATTTCCGATTACATTAATCGTTGGATCTTCGTGACCATTACGAATAACCGTCT
GAATAACAGCAAGATTTACATCAATGGTCGCTTGATCGATCAGAAACCGATTAGCAACCTGGGTAATATCCACGCAAGCAACA
ACATTATGTTCAAATTGGACGGTTGCCGCGATACCCATCGTTATATCTGGATCAAGTATTTCAACCTGTTTGATAAAGAACTG
AATGAGAAGGAGATCAAAGATTTGTATGACAACCAATCTAACAGCGGCATTTTGAAGGACTTCTGGGGCGATTATCTGCAATA
CGATAAGCCGTACTATATGCTGAACCTGTATGATCCGAACAAATATGTGGATGTCAATAATGTGGGTATTCGTGGTTACATGT
ATTTGAAGGGTCCGCGTGGCAGCGTTATGACGACCAACATTTACCTGAACTCTAGCCTGTACCGTGGTACGAAATTCATCATT
AAGAAATATGCCAGCGGCAACAAAGATAACATTGTGCGTAATAACGATCGTGTCTACATCAACGTGGTCGTGAAGCGTAAAGA
GTACCGTCTGGCGACCAACGCTTCGCAGGCGGGTGTTGAGAAAATTCTGAGCGCGTTGGAGATCCCTCGTGTCCGTCGTCTGA
GCCAAGTCGTGGTTATGAAGAGCAAGAACGACCAGGGTATCACTAACAAGTGCAAGATGAACCTGCAAGACCGTCGTGGTAAC
GACATCGGCTTTATTGGTTTCCACCAGTTCAACAATATTGCTAAACTGGTAGCGAGCAATTGGTACAATCGTCAGATTGAGCG
CCGTAGCCGTCGTTTGGGCTGTAGCTGGGAGTTTATCCCGGTCGATGATGGTTGGGGCGAACGTCCGCTG
SEQ ID NO: 21 (Polypeptide Sequence of Modified BoNT/A ″Cat-D″)
MPFVNKQFNYKDPVNGVDIAYIKIPNAGQMQPVKAFKIHNKIWVIPERDTFTNPEEGDLNPPPEAKQVPVSYYDSTYLSTDNE
KDNYLKGVTKLFERIYSTDLGRMLLTSIVRGIPFWGGSTIDTELKVIDTNCINVIQPDGSYRSEELNLVIIGPSADIIQFECK
SFGHEVLNLTRNGYGSTQYIRFSPDFTFGFEESLEVDTNPLLGAGKFATDPAVTLAHELIHAGHRLYGIAINPNRVFKVNTNA
YYEMSGLEVSFEELRTFGGHDAKFIDSLQENEFRLYYYNKFKDIASTLNKAKSIVGTTASLQYMKNVFKEKYLLSEDTSGKFS
VDKLKFDKLYKMLTEIYTEDNFVKFFKVLNRKTYLNFDKAVFKINIVPKVNYTIYDGFNLRNTNLAANFNGQNTEINNMNFTK
LKNFTGLFEFYKLLCVRGIITSKTKSLDKGYNKALNDLCIKVNNWDLFFSPSEDNFTNDLNKGEEITSDTNIEAAEENISLDL
IQQYYLTFNFDNEPENISIENLSSDIIGQLELMPNIERFPNGKKYELDKYTMFHYLRAQEFEHGKSRIALTNSVNEALLNPSR
VYTFFSSDYVKKVNKATEAAMFLGWVEQLVYDFTDETSEVSTTDKIADITIIIPYIGPALNIGNMLYKDDFVGALIFSGAVIL
LEFIPEIAIPVLGTFALVSYIANKVLTVQTIDNALSKRNEKWDEVYKYIVTNWLAKVNTQIDLIRKKMKEALENQAEATKAII
NYQYNQYTEEEKNNINFNIDDLSSKLNESINKAMININKFLNQCSVSYLMNSMIPYGVKRLEDFDASLKDALLKYIYDNRGTL
IGQVDRLKDKVNNTLSTDIPFQLSKYVDNQRLLSTFTEYIKNIINTSILNLRYESNHLIDLSRYASKINIGSKVNFDPIDKNQ
IQLFNLESSKIEVILKNAIVYNSMYENFSTSFWIRIPKYFNSISLNNEYTIINCMENNSGWKVSLNYGEIIWTLQDTQEIKQR
VVFKYSQMINISDYINRWIFVTITNNRLNNSKIYINGRLIDQKPISNLGNIHASNNIMFKLDGCRDTHRYIWIKYFNLFDKEL
NEKEIKDLYDNQSNSGILKDFWGDYLQYDKPYYMLNLYDPNKYVDVNNVGIRGYMYLKGPRGSVMTTNIYLNSSLYRGTKFII
KKYASGNKDNIVRNNDRVYINVVVKRKEYRLATNASQAGVEKILSALEIPRVRRLSQVVVMKSKNDQGITNKCKMNLQDRRGN
DIGFIGFHQFNNIAKLVASNWYNRQIERRSRRLGCSWEFIPVDDGWGERPL

### EXAMPLES

### Materials and Methods

Animal Model

Male domestic pigs weighing 11-13 kg were used in the following study. The pig is a suitable model for studying the treatment of post-operative surgical pain as porcine skin shares similarities with human skin in terms of structure, thickness, innervation, pigmentation, collagen and lipid composition, wound-healing and immune responses.

Reconstitution of Dysport

Dysport was provided in vials containing 500U. For dosing, vials of 500U were reconstituted with saline (0.9% NaCl). Subsequent dilutions were done with saline according to the testing doses as follows:

2.5 ml of saline was drawn with 3 ml syringe+21 G needle and transferred to the Dysport 500U vial; the concentration was 200U/ml=400U/2 ml; the vial was gently swirled until material was dissolved. Each vial was tilted side-to-side 2-3 times (to ensure solution homogeneity); pigs were dosed with 2 ml using 2 syringes of 1 ml connected to a 30 G needle. This solution was used for dosing of 400U.

Preparation for 200U/2 ml Dose:

Dysport was reconstituted as explained above; 3 ml syringe and 21 G needle were used to draw 2 ml of reconstituted Dysport 500U; 3 ml syringe and 21 G needle were used to draw 2 ml of saline; a vacutainer vial was used to mix the 2 solutions mentioned above; the mixed solution was tilted side-to-side 5-6 times (to ensure solution homogeneity); the pigs were dosed with 2 ml using 2 syringes of 1 ml connected to a 30 G needle.

Preparation for 100U/2 ml Dose:

Dysport was reconstituted as explained above; 3 ml syringe and 21 G needle were used to draw 2 ml of reconstituted Dysport 500U; 3 ml syringe and 21 G needle were used to draw 2 ml of saline; a vacutainer vial was used to mix the 2 solutions mentioned above; the mixed solution was tilted side-to-side 5-6 times (to ensure solution homogeneity); 3 ml syringe and 21 G needle were used to draw 2 ml of prepared solution from the vacutainer vial; 3 ml syringe and 21 G needle were used to draw 2 ml of saline; a new vacutainer was used to mix the 2 solutions mentioned above; the mixed solution was tilted side-to-side 5-6 times (to ensure solution homogeneity); pigs were dosed with 2 ml using 2 syringes of 1 ml connected to 30 G needle.

Induction of Post-Operative Surgical Pain

Pigs were anesthetized by an isoflurane/oxygen mixture, which was delivered through a facemask. The area of the incision was cleaned using Septol and Polydine (Iodo-Vit) solution. A 6-7 cm long skin incision was made in the left flank, towards the caudal end of the pig and 3 cm lateral to the spine line (Day 1) or a 7 cm long skin incision was made in the left leg. Then the fascia was cut and the muscle was retracted (Castel et al., Characterization of a porcine model of post-operative pain, Eur. J. Pain. 2014, 18(4), 496-505; incorporated herein by reference). The sub-cutis was then sutured with 3-0 Vicryl thread. The skin was sutured with 3-0 silk thread using continuous suturing methods. Following the incision closure and material injection, the pigs received antibiotic (Marbocyl 10%). The area of the incision was covered with the thin layer of Syntomicine 3%. The animals were kept under anesthesia for the duration of the surgery and dosing (about 20 minutes). Post-surgery the animals were returned to their pens for recovery and observation.

Treatment

Intradermal peri-operative administration of Dysport

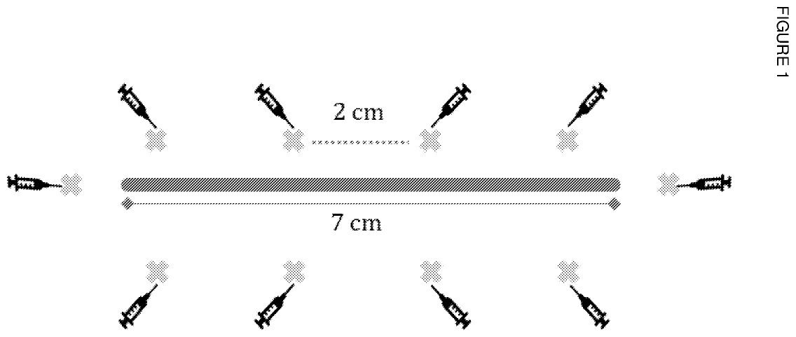

For peri-operative administration, animals were injected just after suturing the incision made in the left flank. Dysport (test item), saline (negative control) or the reference compound Exparel (positive control) were injected intradermally (or subcutaneously for Exparel) using 30G needles attached to 1 ml syringes and into 10 sites around the incision. Each site was injected with a fixed dosing volume and fixed dosing level. In more detail, 4 sites along each side (e.g. 8 sites) of a 7 cm horizontal incision/suture in the left flank were injected (at 2 cm intervals), as were sites at each end of the incision/suture in the left flank (see FIG. 1). The following experimental groups were assessed as follows:

| Group | | Number of | | Dosing level | |
|---|---|---|---|---|---|
| Number | Treatment | Animals | Dosing volume | per animal | |
| 1 | Saline | 6 | 200 μL/site of | Saline | |
| | | | injection | | |
| 2 | Exparel | 6 | 20 ml | 266 | mg |
| 3 | Dysport | 6 | 200 μL/site of | 100 | U |
| | | | injection | | |
| 4 | Dysport | 6 | 200 μL/site of | 200 | U |
| | | | injection | | |
| 5 | Dysport | 6 | 200 μL/site of | 400 | U |

injection

Intradermal Pre-Operative Administration of Dysort

For pre-operative injections, as animals were injected before the incision (either in the left flank or left leg of the pig) with a fixed total volume of 2 ml, the location of the further incision was tattooed first. Dysport (test item) or saline (negative control) were injected intradermally using 30G needles attached to 1 ml syringes and into 10 sites around the incision. Each site was injected with a fixed dosing volume and fixed dosing level. Administrations were performed either at 15 days, 5 days or 1 day prior to surgery. The following experimental groups (when an incision was made in the left flank of the pig) were assessed as follows:

| | | | | | Dosing |
|---|---|---|---|---|---|
| | | | | | day vs. |
| Group | | No. of | | Dosing level | operation |
| Number | Treatment | Animals | Dosing volume | per animal | day |
| 1 | Dysport | 6 | 200 μL/site of | Saline | −15 |
| | | | injection | | |
| 2 | Saline | 6 | 200 μL/site of | 200 U/pig as 10 | |
| | | | injection | sites injected | |
| | | | | with 20 U | |
| 3 | Dysport | 6 | 200 μL/site of | Saline | −5 |
| | | | injection | | |
| 4 | Saline | 6 | 200 μL/site of | 200 U/pig as 10 | |
| | | | injection | sites injected | |
| | | | | with 20 U | |
| 5 | Dysport | 6 | 200 μL/site of | Saline | −1 |
| | | | injection | | |
| 6 | Saline | 6 | 200 μL/site of | 200 U/pig as 10 | |
| | | | injection | sites injected | |
| | | | | with 20 U | |

The following experimental groups (when an incision was made in the left leg of the pig) were assessed as follows:

| | | | | | Dosing |
|---|---|---|---|---|---|
| | | | | | day vs. |
| Group | | No. of | | Dosing level | operation |
| Number | Treatment | Animals | Dosing volume | per animal | day |
| 1 | Saline | 6 | 200 μL/site of | Saline | −15 |
| | | | injection | | |
| 2 | Dysport | 5 | 200 μL/site of | 200 U/pig as 10 | |
| | | | injection | sites injected | |
| | | | | with 20 U | |
| 3 | Exparel | 6 | 20 ml | 266 mg | 1 |

Administration of Dysport Via Intradermal. Intramuscular or Subcutaneous Routes

As animals were injected 15 days before the incision, the location of the further incision was tattooed. Dysport (test item) or saline (negative control) were injected using 30G needles attached to 1 ml syringes and into 10 sites around the incision. Each site was injected with a fixed dosing volume and fixed dosing level. Administrations were performed either via the intradermal, subcutaneous or intramuscular route.

The following experimental groups were assessed as follows:

| | | | | Dosing day |
|---|---|---|---|---|
| | | | Dosing | vs. |
| Group | | Route of | volume and | operation |
| Number | Treatment | administration | dosing level | day |
| 1 | Dysport | Intramuscular | 200 U/2 ml/ | −15 |
| 2 | Saline | | pig split into | |
| | | | 10 sites of 200 | |
| | | | μL | |
| 3 | Dysport | Intradermal | 200 U/2 ml/ | −15 |
| 4 | Saline | | pig split into | |
| | | | 10 sites of 200 | |
| | | | μL | |
| 5 | Dysport | Subcutaneous | 200 U/2 ml/ | −15 |
| 6 | Saline | | pig split into | |
| | | | 10 sites of 200 | |
| | | | μL | |

Von Frey Assay

Von Frey assay was performed in healthy, unoperated animals after Dysport/saline injections at 1, 2, 4 and 6 hours post-surgery on day 1 and once-daily for 10 days. Von Frey filaments (Ugo Basile, Italy) were applied at approximately ˜0.5 cm proximal to the incision line to the surface of the flank or leg skin. As the gram number of filaments increases, the force on the flanks' or legs' skin increases. The maximum force used was 60 g. Filaments were applied until the animal withdrew from the stimuli. Each filament was applied 3-5 times. If withdrawal was not achieved, a thicker filament was applied. If a withdrawal was achieved, a thinner filament was applied (thicker or thinner refers to higher/thicker or lower/thinner gram force). By alternating the filament thickness, the force required to achieve withdrawal reaction was determined and recorded. The size and force of the Von Frey filaments are outlined in the table below:

| | | | | | | | | | | |
|---|---|---|---|---|---|---|---|---|---|---|
| Size | 1.65 | 2.36 | 2.44 | 2.83 | 3.22 | 3.61 | 3.84 | 4.08 | 4.17 | 4.31 |
| Force | 0.008 | 0.02 | 0.04 | 0.07 | 0.16 | 0.40 | 060 | 1.00 | 1.40 | 2.00 |
| (g) | | | | | | | | | | |

| | | | | | | | | | Filament in use | |
|---|---|---|---|---|---|---|---|---|---|---|
| Size | 4.56 | 4.74 | 4.93 | 5.07 | 5.18 | 5.46 | 5.88 | 6.10 | 6.45 | 6.65 |
| Force | 4.00 | 6.00 | 8.00 | 10.0 | 15.0 | 26.0 | 60.0 | 100 | 180 | 300 |
| (g) | | | | | | | | | | |

Filament in use

Inclusion criteria: Animals were included in the study if the flank withdrawal force at baseline was ≥26 g (preferably 60 g). After surgery, pain (allodynia) was considered present if flank withdrawal force was ≤10 g. If the animal did not meet this criteria it was excluded from the study. One animal was excluded from the study due to relatively low threshold before operation (≤10 g).

Animals were included in the study if the leg withdrawal force at baseline was ≥13 g. After surgery, pain (allodynia) was considered present if leg withdrawal force was ≤2 g. If the animal did not meet this criteria it was excluded from the study.

Approaching Time test

Prior to the dosing of pigs, the researcher who was conducting the approaching time (AT) test entered the pen for the first time. The normal behaviour of the pigs when someone entering their housing pen is moving away from the intruder and then approaching the person. The more familiar the pigs are with the person and the more comfortable they feel, the less time it takes them to approach. The latency to approach the researcher entering their home-pen was measured in seconds (cut-off time at 120 sec). This test was done in the morning, at least 1 hour post morning feeding (at 6:30 am) before the distress behaviour score and during the habituation period.

Distress Behaviour Score

Following incision, the behaviour of the animals changed. When approached, the animals moved away from the researcher entering their pen, tended to guard the incision side and sometimes used vocalization. This is the main phenomenon observed following this type of surgery, in rare cases the animals became restless or showed an isolation behaviour. The distress behaviour is scored from 0 (normal) to 7 (very distressed). The distress behaviour score test is performed immediately after the approaching time test. The animal general behaviour was monitored in their home pen during the morning period. The distress behaviour score also allows the overall health status of animals to be assessed. The behaviour of the animals was scored by an observer blind to the treatment, where the total score is the sum of all sections shown in the table below.

| Scoring | | |
|---|---|---|
| Section | Parameter | Score |
| Section 1 | Avoiding standing (lying down) | 1 |
| | Standing | 0 |
| Section 2 | Avoiding walking | 1 |
| | Walking | 0 |
| Section 3 | Protecting the incision side while walking | 1 |
| | Acting normal | 0 |
| Section 4 | Moving away when approached by researcher | 1 |
| | Not moving away when approached by researcher | 0 |
| Section 5 | Restlessness | 1 |
| | Normal | 0 |
| Section 6 | Staying in isolation from other animals | 1 |
| | Staying together with other animals | 0 |
| Section 7 | Screaming (high vocalisation) | 1 |
| | Normal vocalisation | 0 |

The assessment of the behaviour score was not done in a particular order but according to the animal's total spontaneous behaviour.

Open Field Test for Locomotion Activity

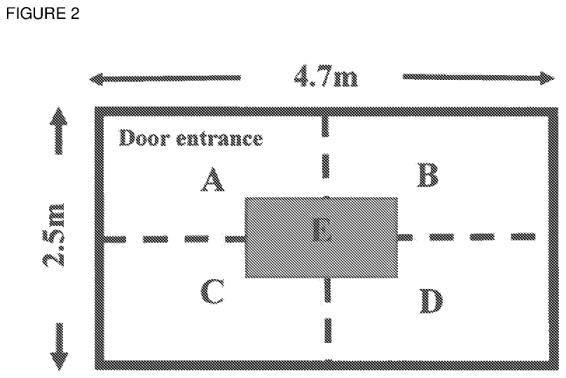

The open field is a rectangle arena 2.5 m wide and 4.7 m long. The walls of the arena are smooth and 1.6 m high. In the morning of the test, the animals from all groups were introduced to the open field individually, one at a time, for a period of 5 minutes (5). The locomotor activity of the animals was recorded using a CCTV camera and analysed with the AnyMaze software (Stoelting Co.). The open field test was performed at the end of behavioural testing performed in the pen (i.e., approaching time, distress behaviour and von Frey). After each open field experiment, the following parameters were analysed: total walking distance (m) and percentages of time spent in the center of the area (Zone E; See FIG. 2).

Distressed animals and animals under pain normally walk closer to the walls of the pen or the open field apparatus. Animals with no distress will not hesitate to enter the center of the open field apparatus.

### Example 1

Peri-Operative Administration of Dysport Provides a Delayed Analgesic and Anxiolytic Effect Post-Surgery (Incision on Left Flank of the Pig)

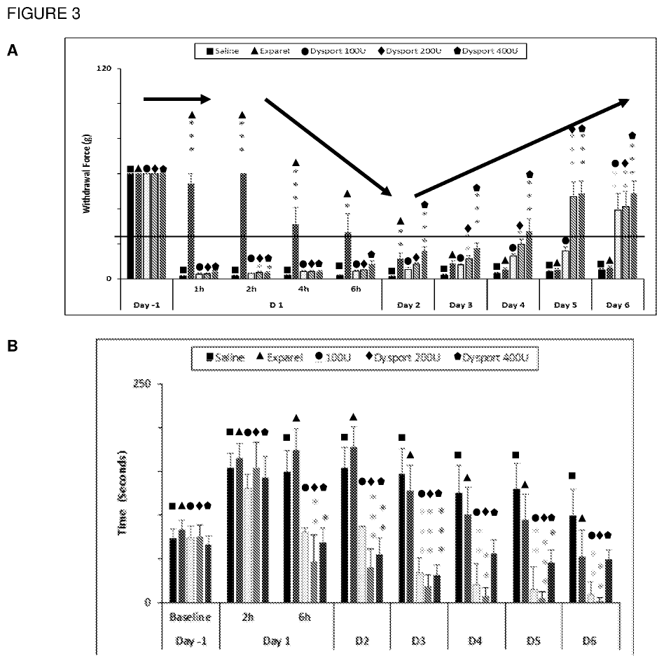

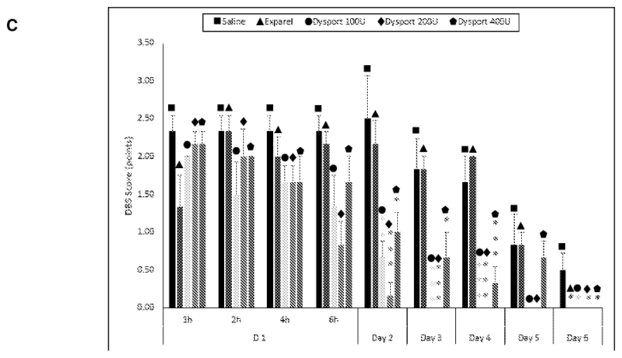

Pigs were administered intradermal injections of either saline, Exparel (266 mg fixed doses) or different concentrations of Dysport immediately after suturing the incision made on the left flank of the pig (that is, peri-operatively). The mechanical sensitivity of pigs was measured by a von Frey assay as an assessment of treatment of post-operative surgical pain. When compared to the saline-treated group, Exparel showed an analgesic effect for a duration of 1 day but showed no effective analgesic activity afterwards. Administration of 400U of Dysport induced a moderate analgesic effect 2 days post-surgery. A greater analgesic effect was induced by 4 days post-surgery when pigs were administered either 200U or 400U of Dysport. All the concentrations of Dysport tested completely suppressed post-operative surgical pain 6 days post-operation. This suggests that Dysport provides an effective and prolonged analgesic effect for treating post-operative surgical pain. This data is illustrated in a bar chart in FIG. 3A.

The latency of pigs to approach their handler was measured. Pigs were administered intradermal injections of either saline, Exparel or different concentrations of Dysport at the time of incision. By 2 hours post-surgery, all treatment groups showed a delay in approaching their handler. By 6 hours, intradermal administration with either 200U or 400U of Dysport reduced the time taken for pigs to approach their handler, with these effects continuing for up to 5 days post-surgery, suggesting a potential reduction in post-operative distress and anxiety-like reactivity. Pigs treated with either saline or Exparel failed to show any improvements in approaching their handler, suggesting that these treatments do not reduce post-operative distress and anxiety-like reactivity. This data is illustrated in FIG. 3B.

The distress behaviour score of pigs was measured. Pigs administered either 100U, 200U or 400U of Dysport showed a reduction in their distress behaviour score by 2 days post-surgery, unlike saline and Exparel treated groups. This data is illustrated in FIG. 3C.

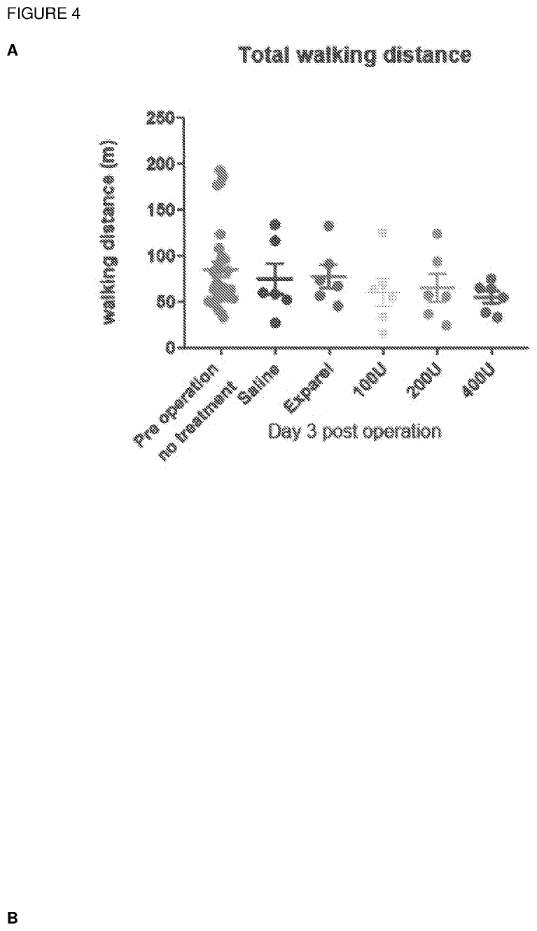

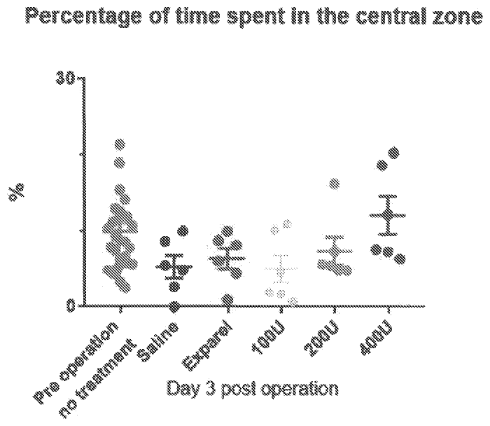

The open field test showed there was no difference between the total distance that the animals walked prior to surgery and post-surgery following saline treatment. Treatment with Exparel or Dysport did not affect the total walking distance at 3 days post dosing, suggesting that there was no change in the animals' motor function following the surgery. This data is illustrated in FIG. 4A. Animals treated with 400U Dysport spent more time in the center of the open field apparatus, although this difference was not statistically significant (see FIG. 4B).

### Example 2

Pre-Operative Administration of Dysport Induces a Faster Analgesic Effect and Suppresses the Emergence of Post-Operative Distress and Anxiety-Like Reactivity when a Surgical Incision is Made in the Left Flank of the Pig

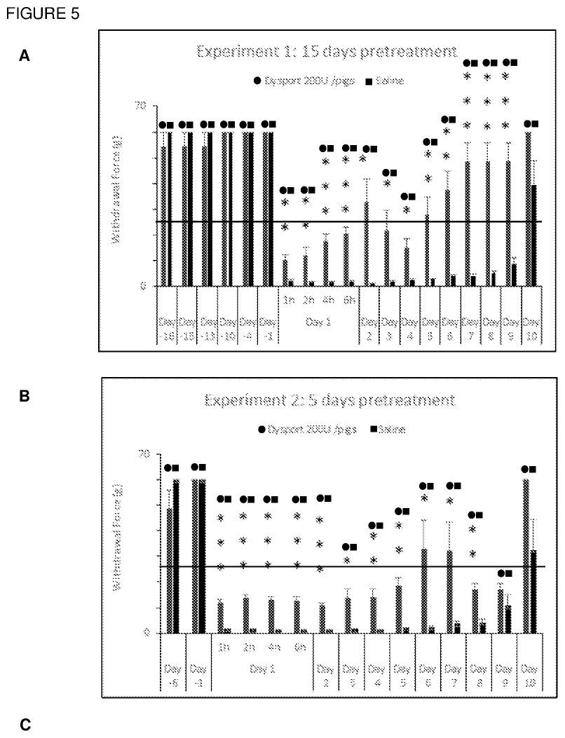

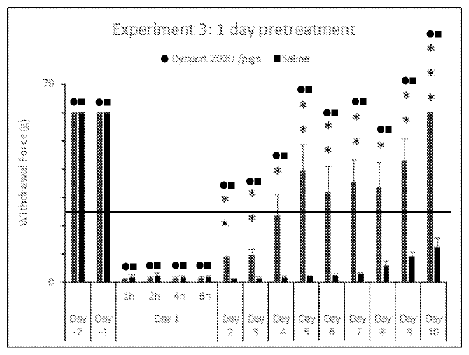

As the peri-operative administration of Dysport showed a delay in inducing an analgesic effect, the analgesic and anxiolytic effects of pre-operative administration of Dysport were measured. Pigs were administered intradermal injections of either saline or 200U of Dysport, at 15 days (see FIG. 5A), 5 days (see FIG. 5B) or 1 day (see FIG. 5C) prior to surgery (incision in the left flank of the pig). By using a Von Frey assay, the fastest analgesic effect was observed when Dysport was administered 15 days prior to surgery, where post-operative surgical pain was reduced by 1 day post-surgery. In comparison, when Dysport was administered 5 days prior to surgery, post-operative surgical pain was reduced by 5 days post-surgery.

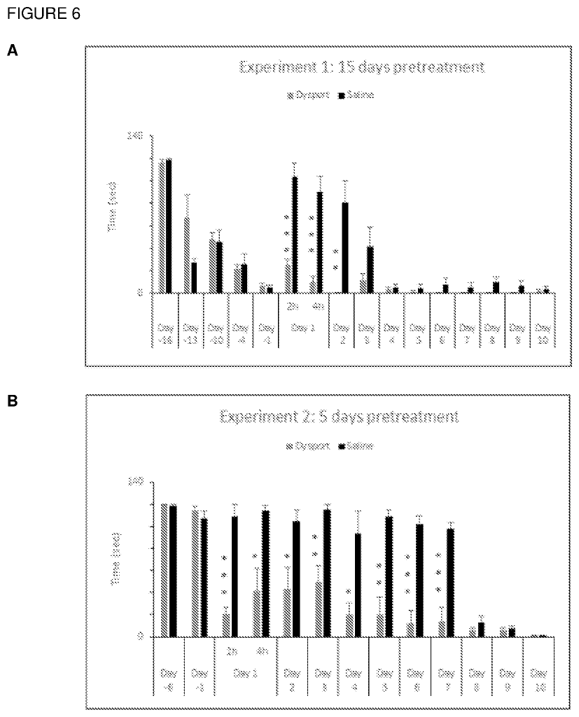

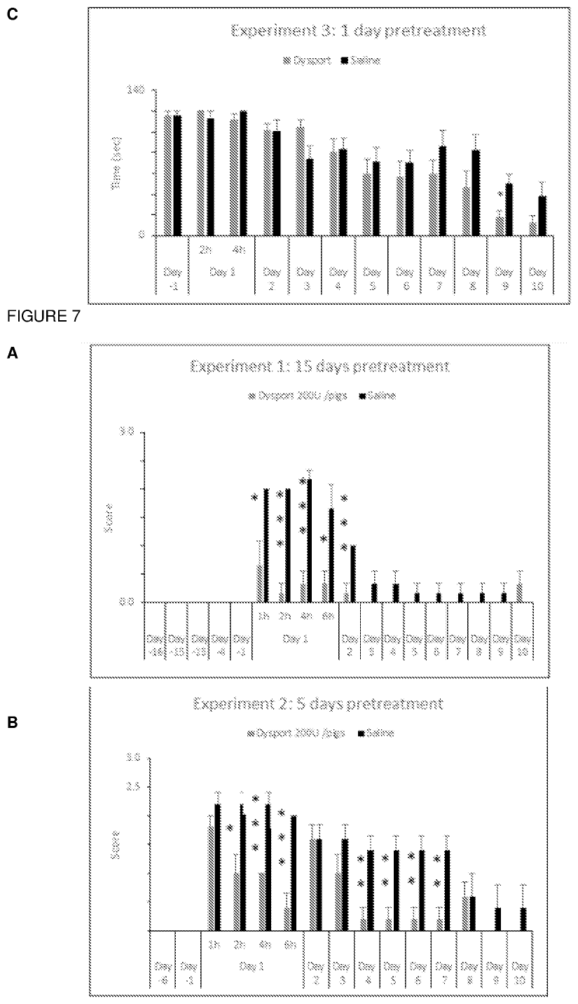

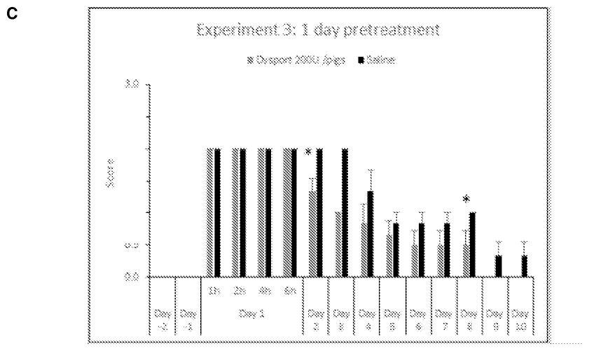

Pigs showed a reduced time to approach their handlers when administered intradermal injections of Dysport 15 or 5 days prior to surgery (see FIG. 6). Similarly, pigs showed a reduced distress behaviour score when administered intradermal injections of Dysport 15 or 5 days prior to surgery (see FIG. 7). The administration of Dysport 1 day prior to surgery did not induce as effective anxiolytic effects. This suggests that the pre-operative administration of Dysport 15 or 5 days prior to surgery fully prevents the emergence of post-operative distress and anxiety-like reactivity.

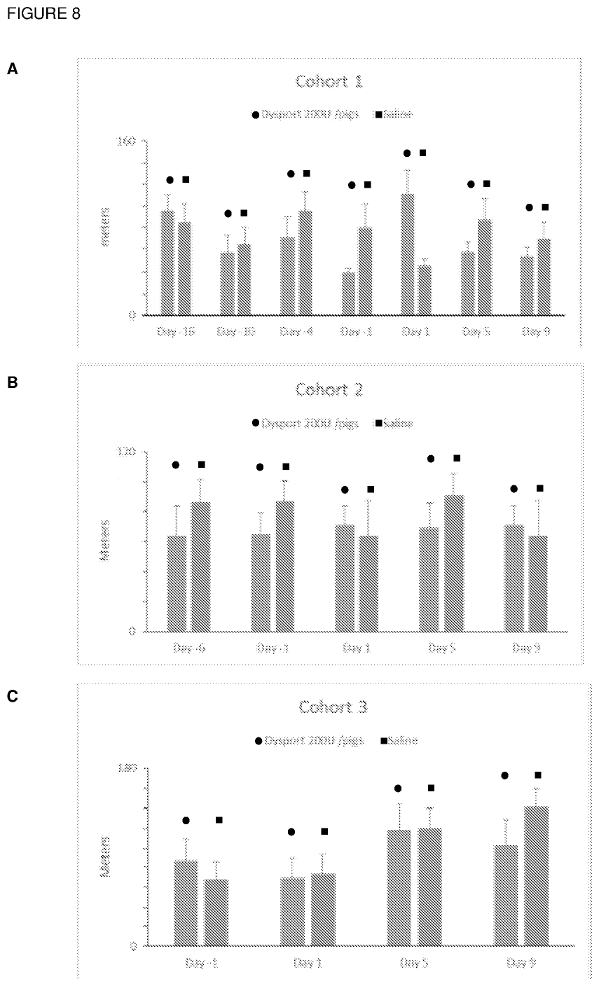

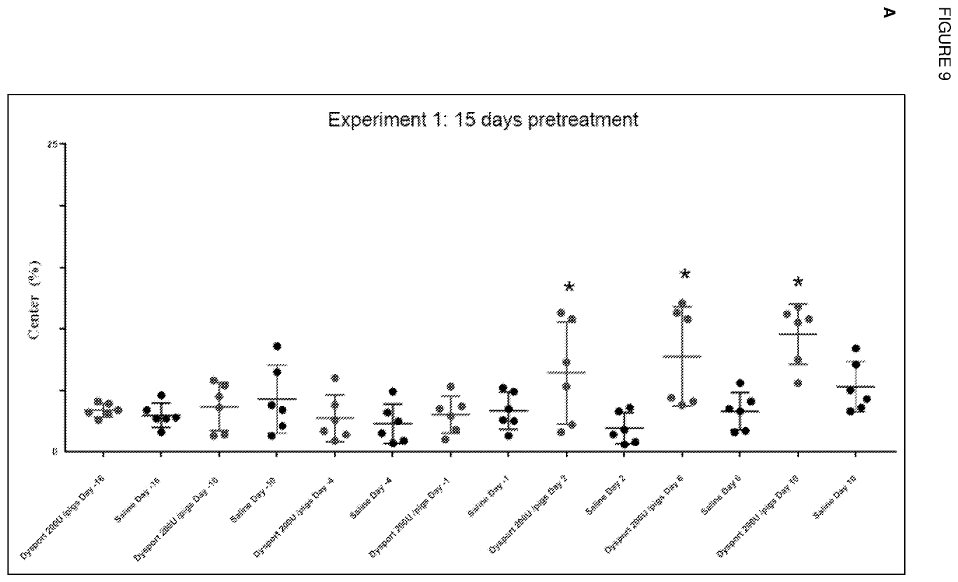

None of the treatment groups (intradermal injections of Dysport 15 days, 5 days or 1 day prior to surgery) showed a difference in their post-operative total walking distance (see FIG. 8), suggesting that muscle activity was unaffected and there was no systemic spread of the toxin.

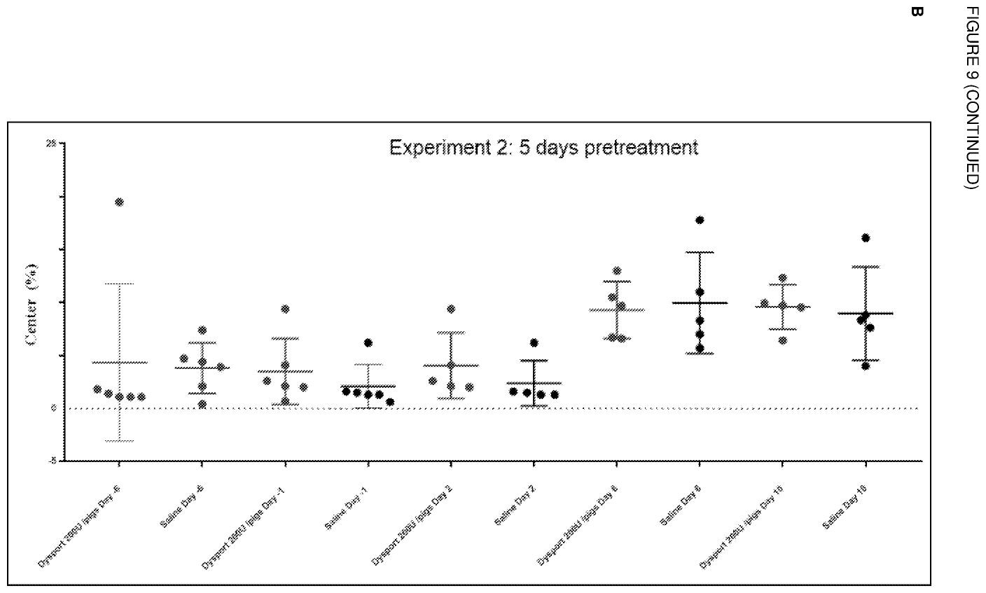

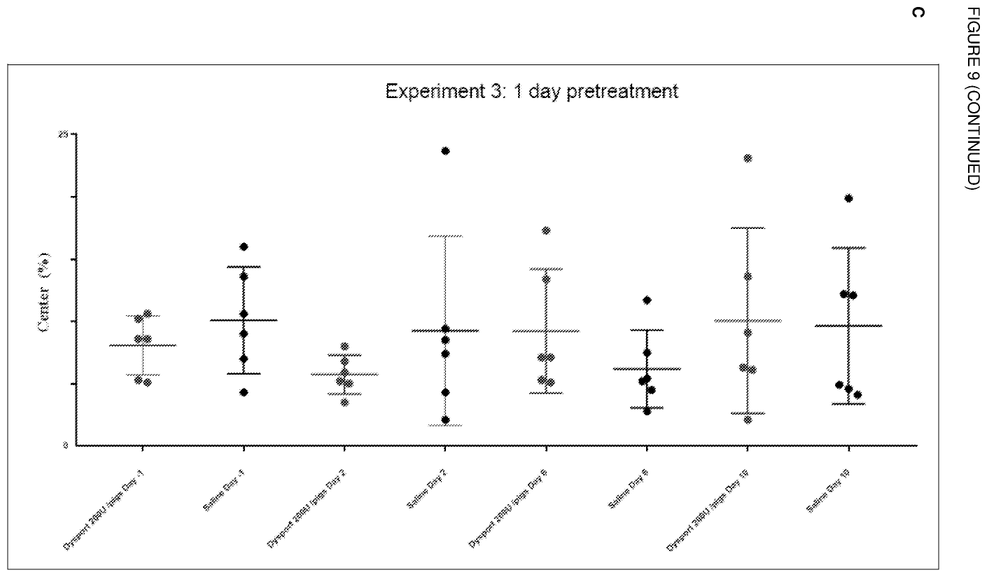

The percentage of time spent in the center of the open-field apparatus by saline-injected animals was similar before and following the surgery. Animals treated with Dysport 15 days prior to surgery spent more time in the center of the open filed apparatus (see FIG. 9A). There was no difference in the percentage of time spent in the central zone between saline and Dysport treated animals, when administered either at 5 days or 1 day prior to surgery (see FIG. 9B and FIG. 9C).

### Example 3

Intradermal Administration of Dysport Provides an Advantageous Route for Mitigating Post-Operative Surgical Pain and Suppressing the Emergence of Post-Operative Anxiety

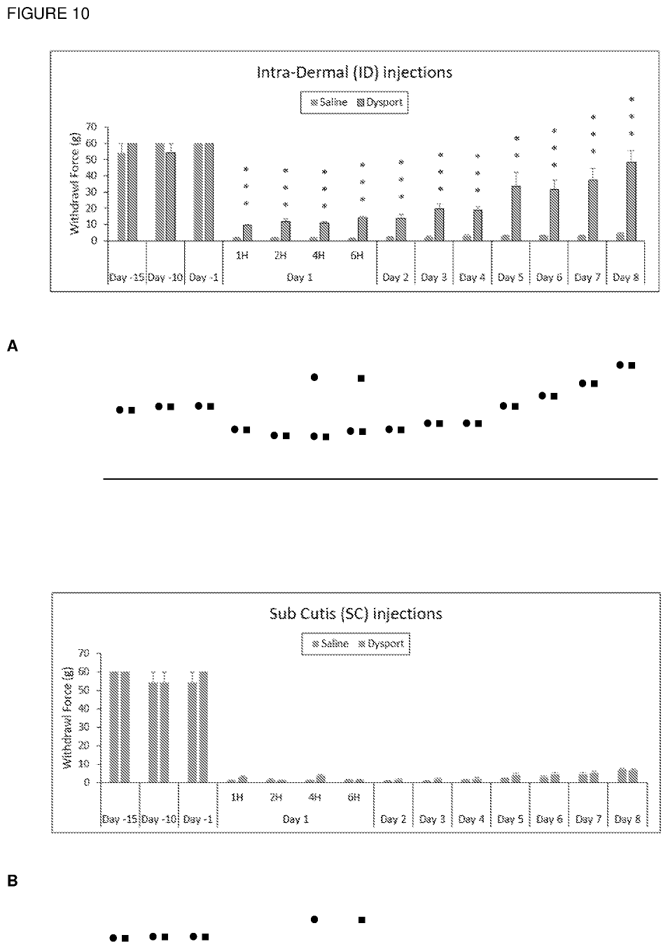

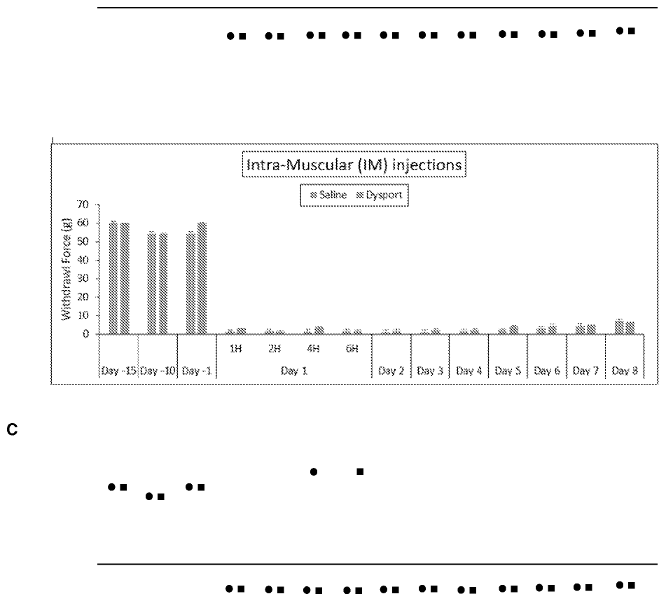

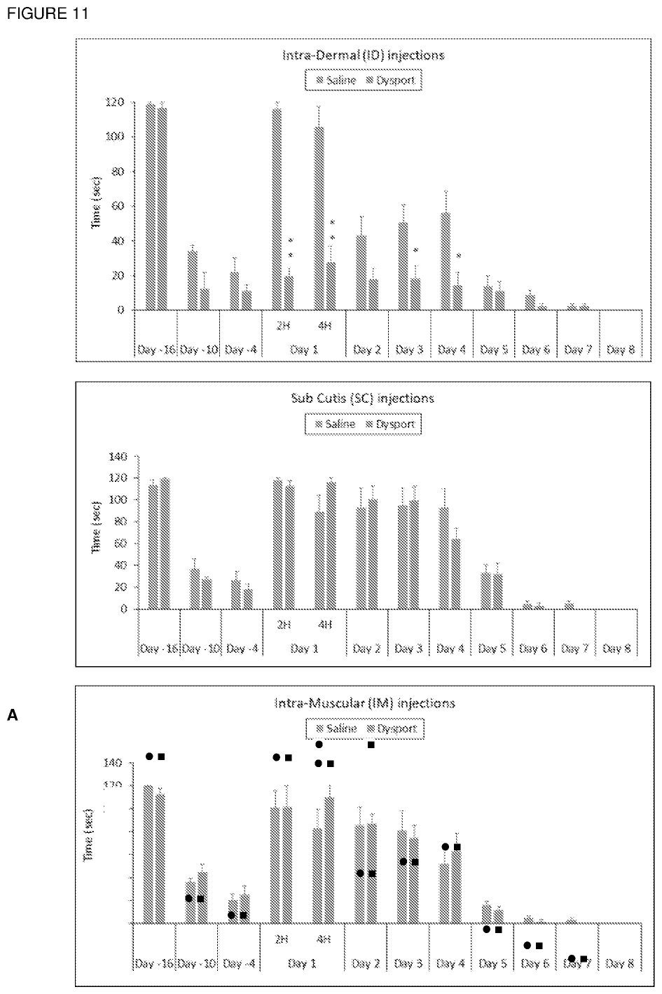

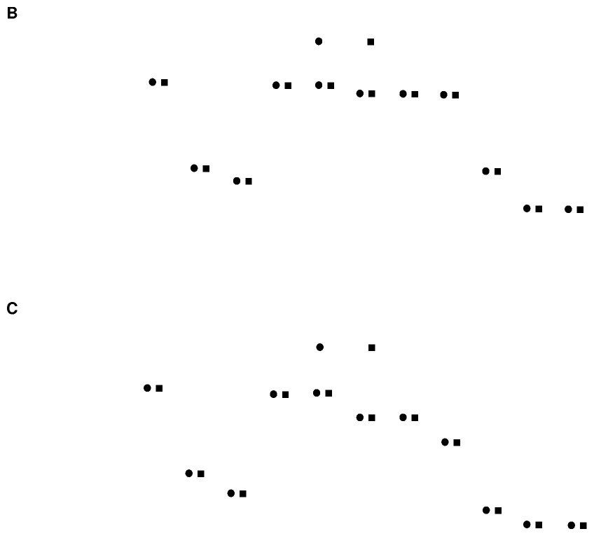

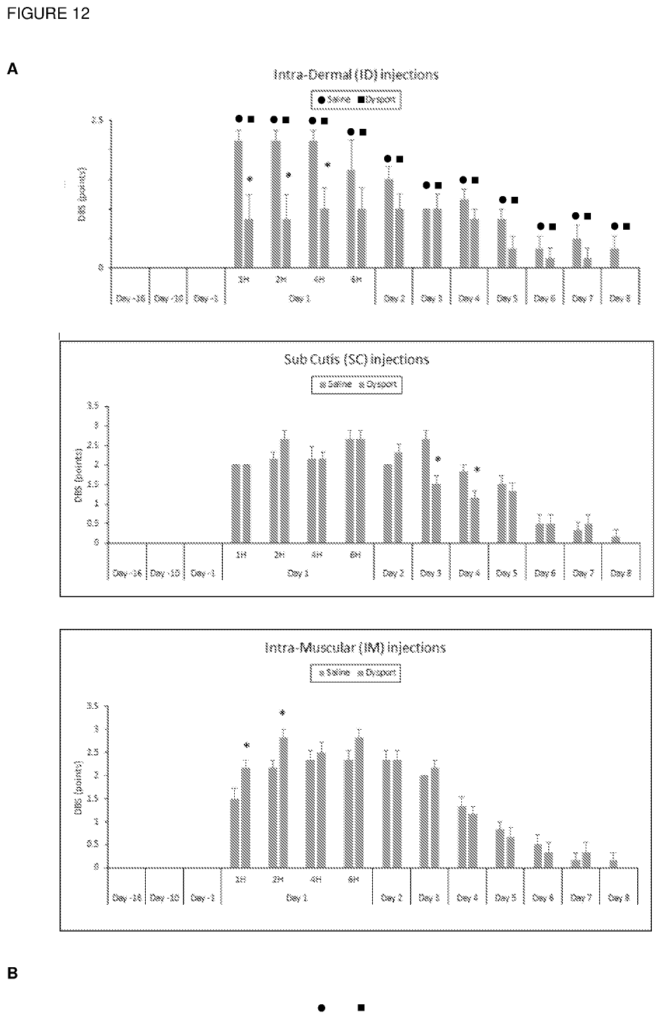

Different routes of 200U of Dysport administration (intradermal, subcutaneous and intramuscular injections) 15 days prior to surgery were assessed for their ability to induce analgesic and anxiolytic effects post-operatively. Surprisingly, intradermal administration provided for better results than the alternative routes (indeed, it was generally observed that only the intradermal route of Dysport administration showed a rapid, analgesic effect (see FIG. 10). Both the subcutaneous and intramuscular routes of Dysport administration showed little to no effects on analgesic activity. Pigs showed a reduced time to approach their handler and a reduced distress behaviour score when administered intradermal injections of Dysport 15 days prior to surgery (see FIG. 11 and FIG. 12). This suggests that the intradermal route of administration is effective at relieving post-operative surgical pain and preventing the full emergence of post-operative distress and anxiety-like reactivity.

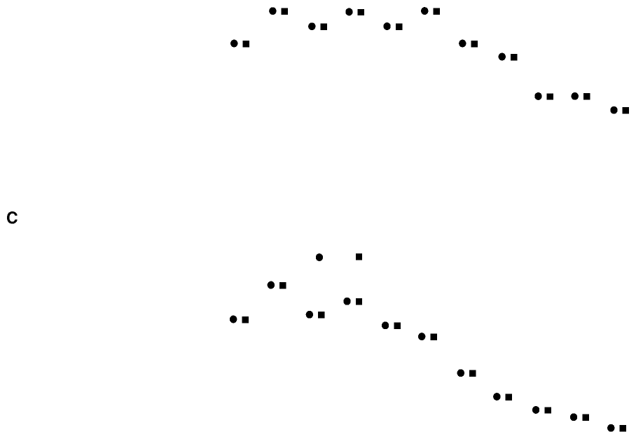

The walking distance recorded post-surgery was the same as that recorded prior to the surgery in all saline groups. Furthermore, treatment with Dysport and its route of administration (intradermal, subcutaneous or intramuscular routes) did not affect the total walking distance post-surgery (see FIG. 13).

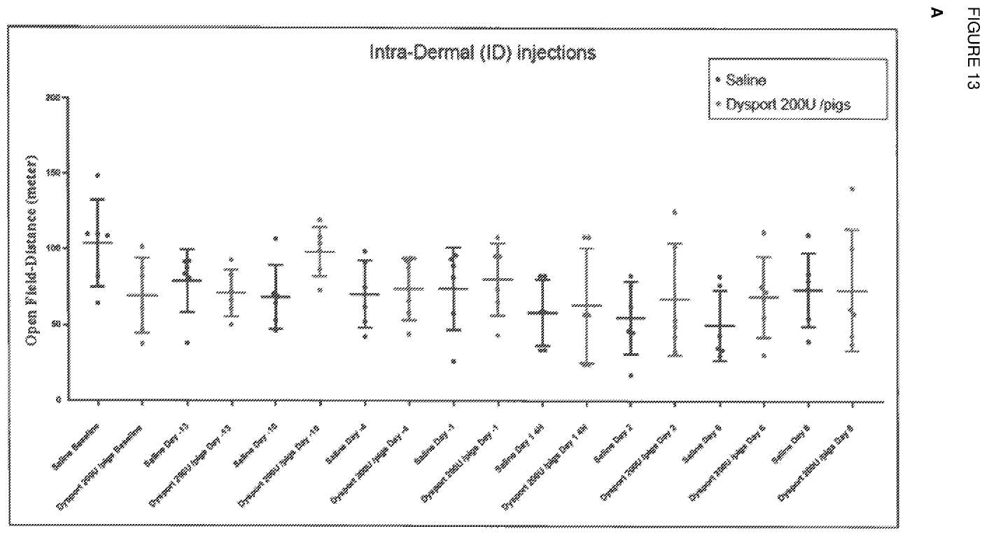

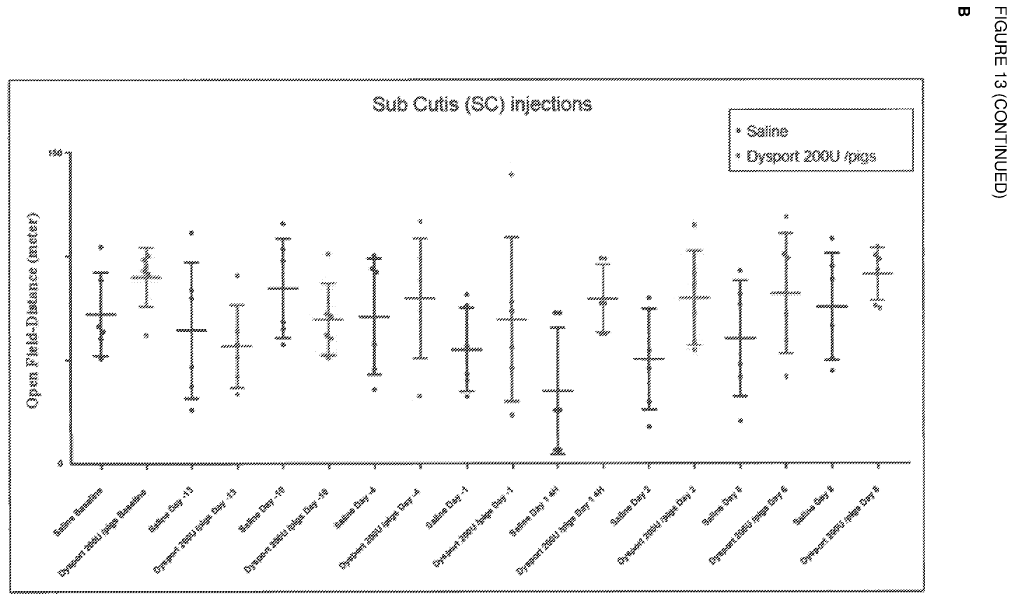

The percentage of time spent in the center of the open field apparatus by saline-injected animals was similar before and after surgery. There was no difference in the percentage of time animals spent in the center of the open field apparatus between the different administration routes (see FIG. 14).

### Example 4

SNAP-25 Cleavage Occurs at a Site Distal to Dysport Injection, in the Ipsilateral Dorsal Horn of the Spinal Cord

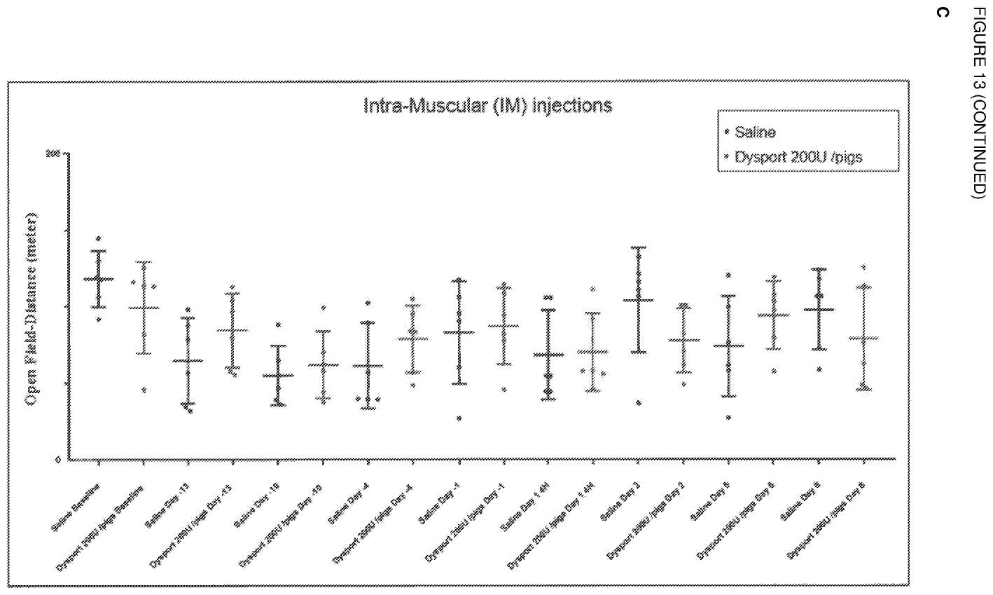

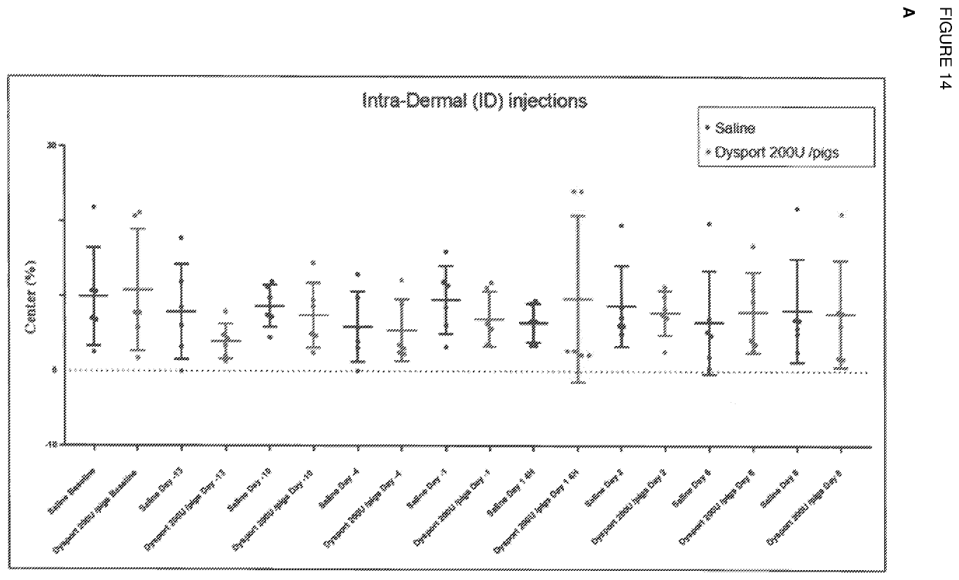

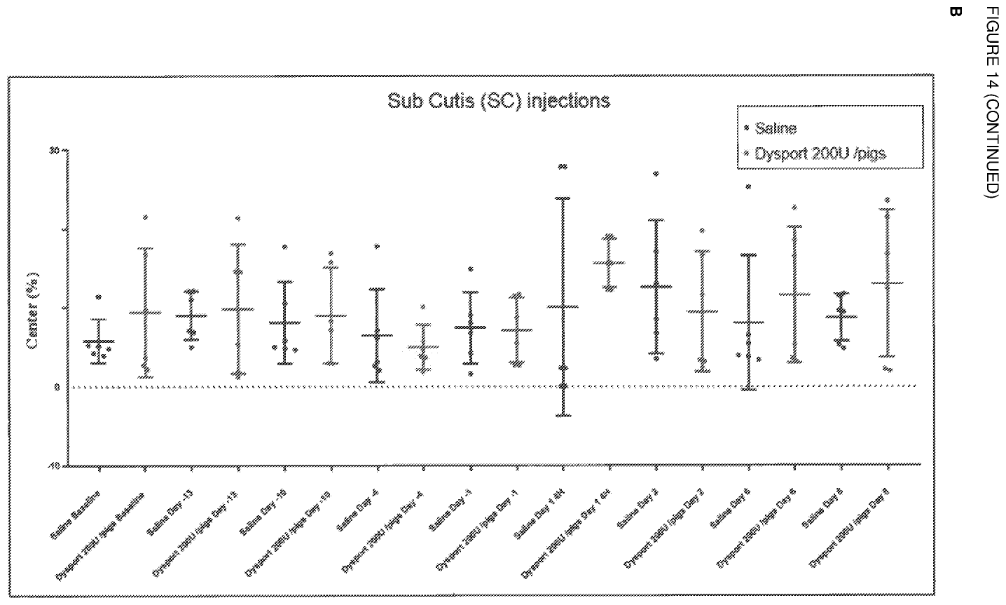

To assess the mechanism of action of Dysport (intradermal injection), immunohistochemistry was performed on both tissue samples at the site of surgical incision (left flank of the pig) and at the spinal cord. Cleaved SNAP-25 was not detected in the nerves of skin samples (see FIG. 15). Unexpectedly, cleaved SNAP-25 was visualised in the ipsilateral dorsal horn of the spinal cord (see FIG. 16), indicative of BoNT/A activity in the spinal cord and that post-operative surgical pain/anxiety control may be provided via a central effect in the spinal cord. This also highlights that Dysport may be administered directly into the spinal cord via intrathecal administration.

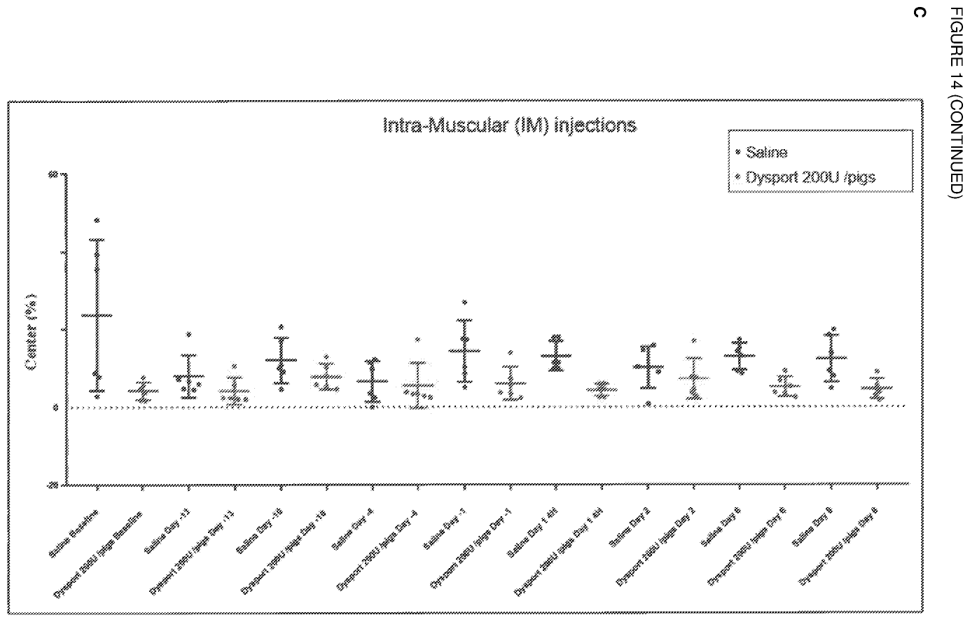

The expression levels of two neuropeptides involved in pain modulation, calcitonin gene related peptide (CGRP) and Substance P, were assessed in the spinal cord by immunohistochemistry staining. Neither neuropeptide showed a difference in their expression level in the spinal cord of pigs treated with Dysport compared to untreated (see FIG. 17).

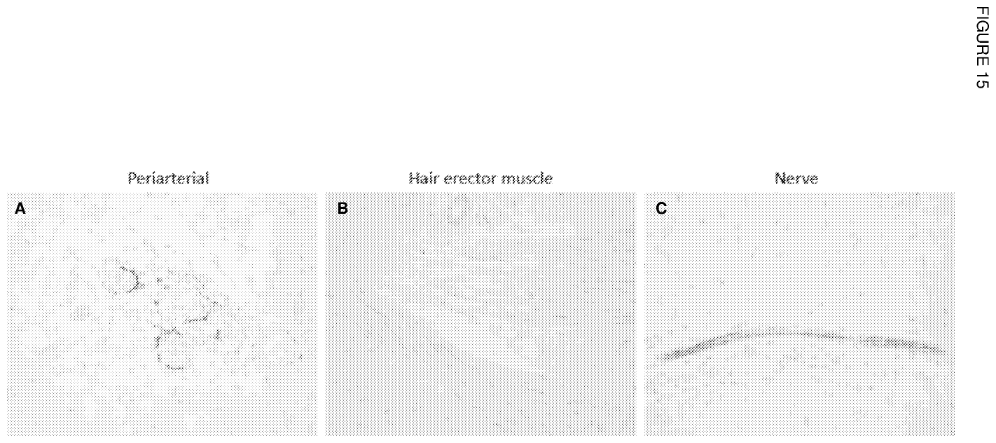

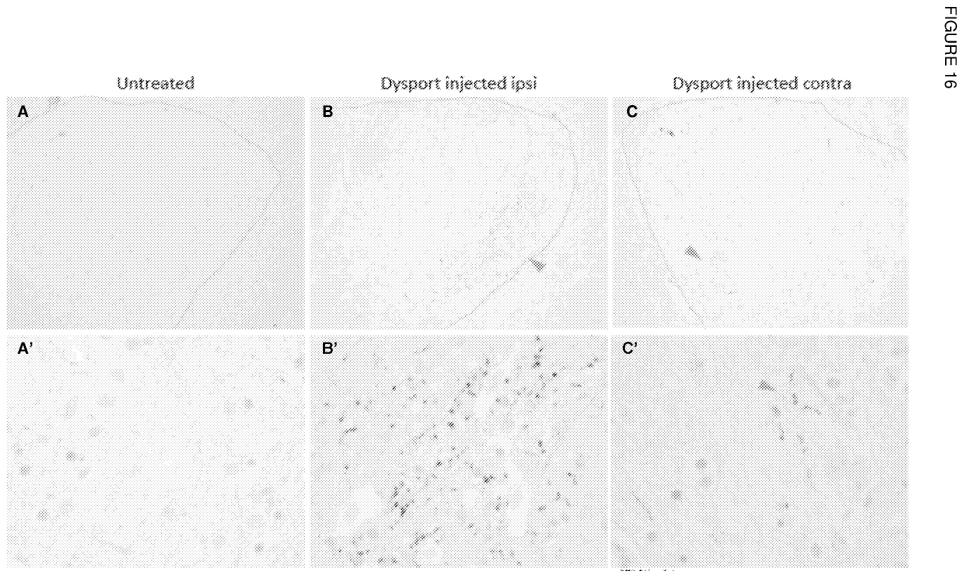

The expression levels of a marker of microglial cell activation, Iba1, were decreased in the spinal cord of pigs treated with Dysport when compared to untreated (see FIG. 18A, B). Similarly, expression levels of a marker of astrocyte activation, GFAP, were decreased in the spinal cord of pigs treated with Dysport when compared to untreated (FIG. 18C, D).

### Example 5

Pre-Operative Administration of Dysport Induces a Fast Analgesic Effect and Suppresses the Emergence of Post-Operative Distress and Anxiety-Like Reactivity when a Surgical Incision is Made in the Left Leg of the Pig

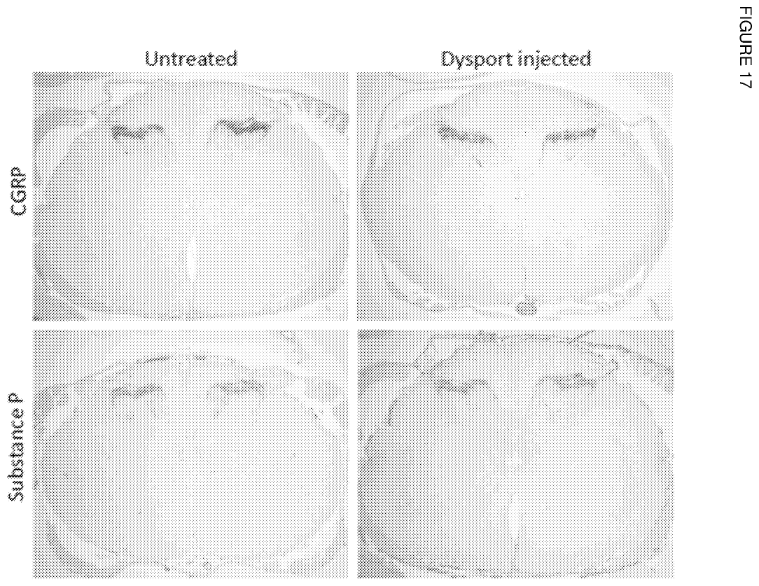

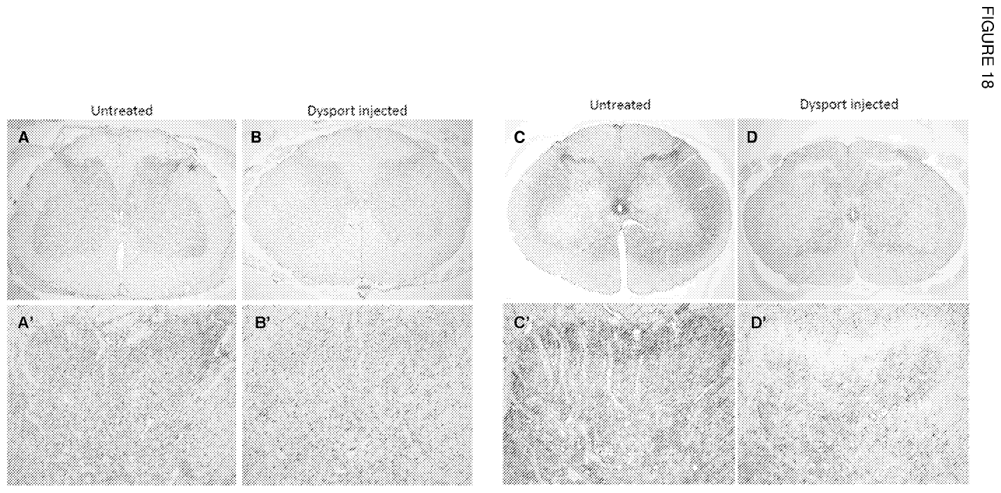

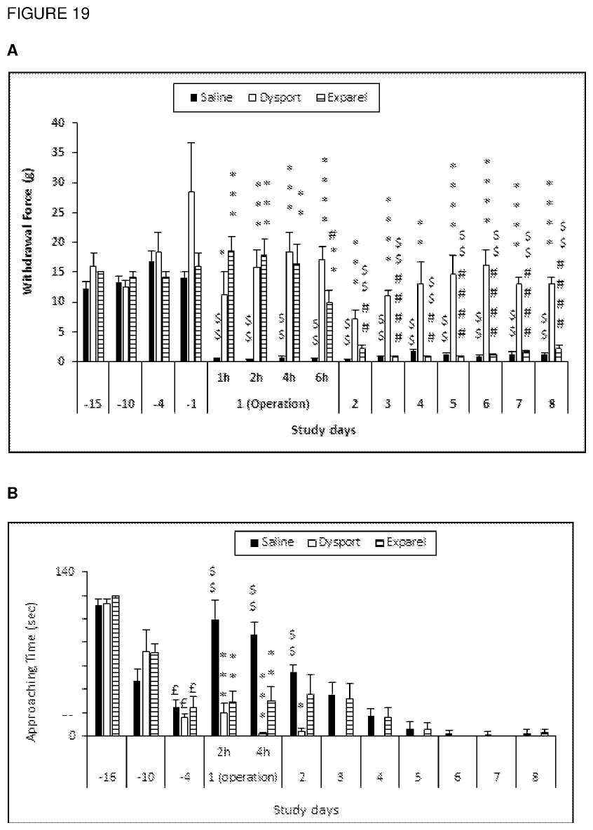

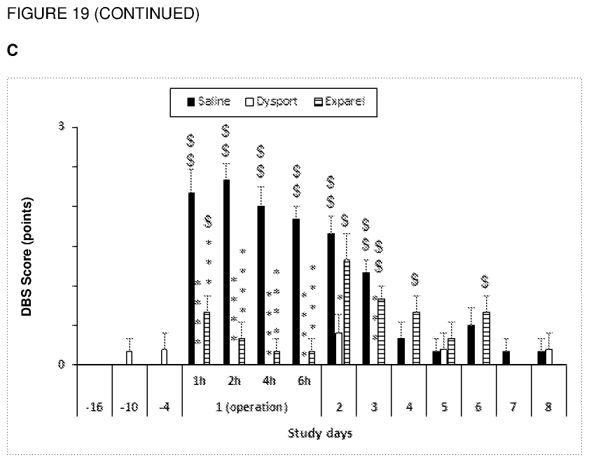

Analgesic and anxiolytic effects of pre-operative administration of Dysport were measured when a different site of surgical incision was made to the pig (surgical incision to the left leg instead of the left flank). Pigs were administered intradermal injections of either saline or 200U of Dysport at 15 days prior to surgery or Exparel on the day of the surgery (day 1) (see FIG. 19A), (surgical incision to the left leg). By using a Von Frey assay, a fast analgesic effect was observed, where post-operative surgical pain was reduced by 1 day post-surgery and a long-lasting reversal of mechanical allodynia was observed by day 4.

Pigs (with a sutured incision in the left leg) showed a reduced time to approach their handlers when administered intradermal injections of Dysport 15 days prior to surgery when compared to administration of saline and Exparel (see FIG. 19B). Similarly, pigs (with a sutured incision in the left leg) showed a reduced distress behaviour score when administered intradermal injections of Dysport 15 days prior to surgery when compared to administration of saline and Exparel (see FIG. 19C). This suggests that the pre-operative administration of Dysport 15 days prior to surgery (incision to the left leg) fully prevents the emergence of post-operative distress and anxiety-like reactivity.

Overall, this experiment provides further support for the fast analgesic and anxiolytic effects when Dysport is administered 15 days prior to surgery.

### Example 6

SNAP-25 Cleavage Occurs in the Ipsilateral Dorsal Horn of the Spinal Cord in Pigs with a Surgical Incision to the Left Leg

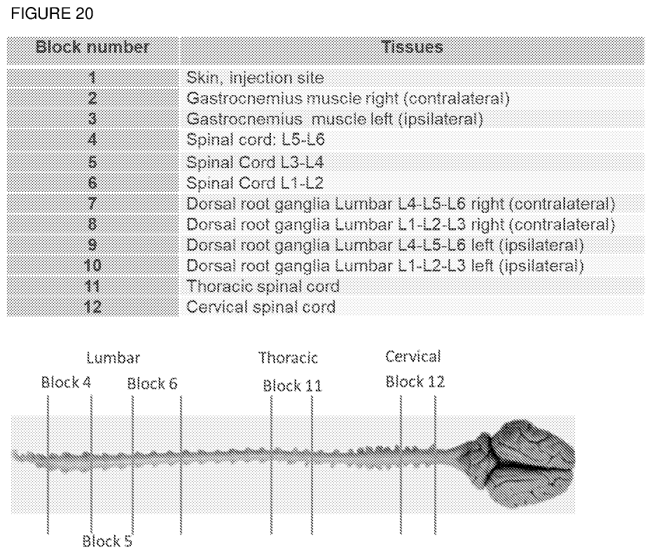

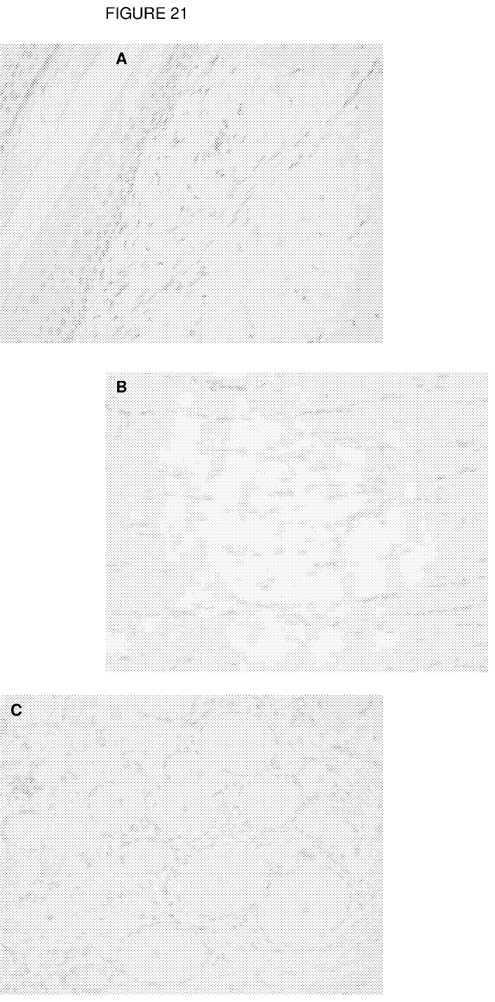

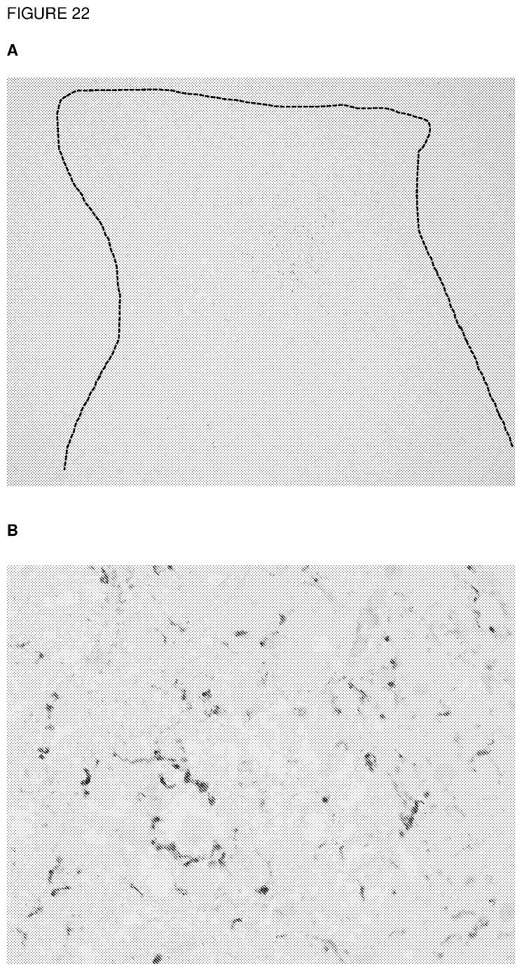

To assess whether a same mechanism of action of Dysport occurred when Dysport was intradermally administered at a different site in the pig, immunohistochemistry was performed on tissue samples at the site of surgical incision (left leg of the pig) and at different regions of the spinal cord (see FIG. 20). Cleaved SNAP-25 was not detected in the nerves of skin samples on samples collected 5-7 days after the incision and injection of Dysport (see FIG. 21). Cleaved SNAP-25 was visualised in the ipsilateral dorsal horn of the spinal cord, specifically in lumbar regions L5-L6 (see FIG. 22), similar to findings in pigs with a surgical incision made to their left flank. These findings are indicative of BoNT/A activity in the spinal cord and that post-operative surgical pain/anxiety control may be provided via a central effect in the spinal cord. The localisation of cleaved SNAP-25 staining in the ipsilateral dorsal horn was different when compared to cleaved SNAP-25 staining in pigs with a surgical incision made to their left flank.

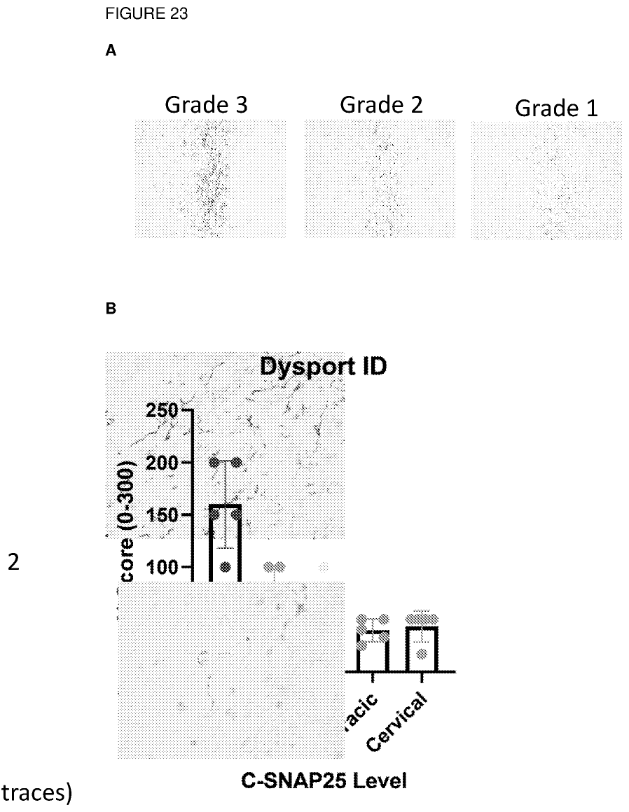

The intensity of cleaved SNAP-25 staining was graded on a scale of 1-3, with grade 0=no cleaved SNAP-25 staining, grade 1=low intensity cleaved SNAP-25 staining, grade 2=average cleaved SNAP-25 intensity staining and grade 3=high intensity cleaved SNAP-25 staining (see FIG. 23A). Based on said grading system, pigs with a surgical incision made to their left leg had lower intensity cleaved SNAP-25 staining in the ipsilateral dorsal horn when compared to pigs with a surgical incision made to their left flank.

The intensity of cleaved SNAP-25 staining was quantified (see FIG. 23B). A H-Score was calculated as a measure of cleaved SNAP-25 staining intensity. The H-Score was calculated by multiplying the % of positive spinal cord sections by the staining intensity in the dorsal horns. In pigs treated with Dysport (and with a surgical incision in the left leg), the highest staining of cleaved SNAP-25 was observed in the spinal cord in lumbar regions L5-L6 (assigned a cleaved SNAP-25 intensity staining of grade 2) when compared to lumbar regions L3-L4 and L1-L2, and the thoracic and cervical regions of the spinal cord (the cervical region had traces of cleaved SNAP-25 staining). There was no evidence of cleaved SNAP-25 staining in saline or Exparel injected pigs.

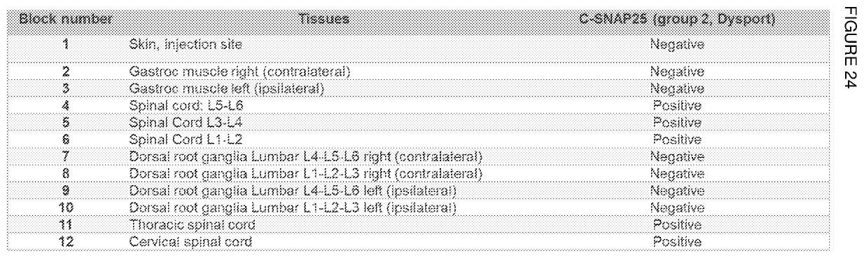

The above immunohistochemistry staining is summarised in FIG. 24 and confirm that SNAP-25 cleavage is observed in localised regions of the spinal cord. Localised regions L5-L6, L3-L4 and L1-L2, and the thoracic and the cervical regions of the spinal cord tested positive for cleaved SNAP-25 staining whilst the remaining tissues (including the skin, at the injection site) tested negative for cleaved SNAP-25 staining.

All publications mentioned in the above specification are herein incorporated by reference. Various modifications and variations of the described methods and system of the present invention will be apparent to those skilled in the art without departing from the scope and spirit of the present invention. Although the present invention has been described in connection with specific preferred embodiments, it should be understood that the invention as claimed should not be unduly limited to such specific embodiments. Indeed, various modifications of the described modes for carrying out the invention which are obvious to those skilled in biochemistry and biotechnology or related fields are intended to be within the scope of the following claims.

1. A clostridial neurotoxin for use in treating post-operative surgical pain in a patient, said method comprising administering to a patient a clostridial neurotoxin more than 5 days prior to surgery and wherein the clostridial neurotoxin is administered:
i) intradermally; or
ii) intrathecally.

2. A method for treating post-operative surgical pain in a patient, said method comprising administering to a patient a clostridial neurotoxin more than 5 days prior to surgery and wherein the clostridial neurotoxin is administered:
i) intradermally; or
ii) intrathecally.

3. The clostridial neurotoxin for use according to claim 1 or the method according to claim 2, wherein the clostridial neurotoxin is administered 6-50 days prior to surgery; preferably 10-20 days prior to surgery.

4. The clostridial neurotoxin for use according to claim 1 or 3 or the method according to claim 2 or 3, wherein the clostridial neurotoxin is administered 14-16 days prior to surgery, preferably about 15 days prior to surgery.

5. The clostridial neurotoxin for use according to any one of claims 1, 3 or 4 or the method according to any one of claims 2-4, wherein the administration of the clostridial neurotoxin substantially reduces post-operative surgical pain perception by the patient and wherein the reduced post-operative surgical pain perception is maintained for 24 hours immediately following surgery.

6. The clostridial neurotoxin for use according to any one of claims 1 or 3-5 or the method according to any one of claims 2-5, wherein substantially all reduced post-operative surgical pain perception is maintained for 3 days immediately following surgery, 5 days immediately following surgery, 7 days immediately following surgery, preferably for 8 days immediately following surgery.

7. The clostridial neurotoxin for use according to claim 5 or 6 or the method according to claim 5 or 6, wherein said reduced level of pain perception observed at a defined time point immediately following surgery is at least 50% of the maximum level of reduced pain perception observed at any time following administration of the clostridial neurotoxin, preferably at least 75% of the maximum level of reduced pain perception observed at any time following administration of the clostridial neurotoxin.

8. A clostridial neurotoxin for use in reducing or suppressing post-operative anxiety, said method comprising administering to a patient a clostridial neurotoxin prior to surgery, wherein the clostridial neurotoxin is administered:
i) intradermally; or
ii) intrathecally.

9. A method for reducing or suppressing post-operative anxiety, said method comprising administering to a patient a clostridial neurotoxin prior to surgery, wherein the clostridial neurotoxin is administered:
i) intradermally; or
ii) intrathecally.

10. The clostridial neurotoxin for use according to claim 8 or the method according to claim 9, wherein the clostridial neurotoxin is administered 5 or more days prior to surgery; preferably wherein the clostridial neurotoxin is administered more than 5 days prior to surgery.

11. The clostridial neurotoxin for use according to claim 8 or 10 or the method according to claim 9 or 10, wherein the administration of the clostridial neurotoxin substantially reduces post-operative anxiety perception by the patient and wherein said reduced post-operative anxiety perception is maintained for 24 hours immediately following surgery.

12. The clostridial neurotoxin for use according to claim 11 or the method according to claim 11, wherein substantially all reduced post-operative anxiety perception is maintained for 2 days immediately following surgery, for 5 days immediately following surgery, for 7 days immediately following surgery, preferably for 9 days immediately following surgery.

13. The clostridial neurotoxin for use according to any one of claims 1, 3-8 or 10-12 or the method according to any one of claims 2-7 or 9-12, wherein the clostridial neurotoxin treats post-operative surgical pain and reduces or suppresses post-operative anxiety.

14. The clostridial neurotoxin for use according to any one of claims 1, 3-8 or 13 or the method according to any one of claims 2-7 or 13, wherein said post-operative surgical pain is caused by surgical intervention and wherein the clostridial neurotoxin is administered at a site distal to the site of surgical intervention.

15. The clostridial neurotoxin for use according to claim 14 or the method according to claim 14, wherein the distal site to the surgical incision is at least 15 cm, 50 cm or 100 cm from the site of surgical intervention.

16. The clostridial neurotoxin for use according to any one of claims 1, 3-8 or 10-15 or the method according to any one of claims 2-7 or 9-15, wherein, following administration, the clostridial neurotoxin travels by retrograde transport to the spinal cord and effects SNARE protein cleavage (SNAP-25 protein cleavage) in said spinal cord.

17. The clostridial neurotoxin for use according to any one of claims 1, 3-8 or 10-16 or the method according to any one of claims 2-7 or 9-16, wherein the clostridial neurotoxin is administered at an intradermal site and wherein minimal or no SNARE protein cleavage (SNAP-25 protein cleavage) by said clostridial neurotoxin is observed at or proximal to said intradermal site following administration of the clostridial neurotoxin.

18. The clostridial neurotoxin for use according to any one of claims 1, 3-8 or 10-16 or the method according to any one of claims 2-7 or 9-16, wherein the clostridial neurotoxin is administered at a site in the intrathecal space of the spinal cord and wherein minimal or no SNARE protein cleavage (SNAP-25 protein cleavage) by said clostridial neurotoxin is observed at or proximal to said site following administration of the clostridial neurotoxin.

19. The clostridial neurotoxin for use according to any one of claims 1, 3-8 or 10-18 or the method according to any one of the claims 2-7 or 9-18, wherein the surgical intervention comprises an incision to the skin and/or fascia and/or muscle, preferably wherein the surgical intervention comprises of an incision to the skin.

20. The clostridial neurotoxin for use according to any one of claims 1, 3-8 or 10-19 or the method according to any one of claims 2-7 or 9-19, wherein the clostridial neurotoxin is a botulinum neurotoxin (BoNT).

21. The clostridial neurotoxin for use according to any one of claims 1, 3-8 or 10-20 or the method according to any one of claims 2-7 or 9-20, wherein the clostridial neurotoxin is botulinum neurotoxin serotype A (BoNT/A).

22. The clostridial neurotoxin for use according to any one of claims 1, 3-8 or 10-21 or the method according to any one of claims 2-7 or 9-21, wherein the post-operative surgical pain is acute post-operative surgical pain.

23. The clostridial neurotoxin for use according to any one of claims 1, 3-8 or 10-22 or the method according to any one of claims 2-7 or 9-22, wherein the post-operative surgical pain is chronic post-operative surgical pain.

24. The clostridial neurotoxin for use according to any one of claims 1, 3-8 or 10-23 or the method according to any one of claims 2-7 or 9-23, wherein said use or said method does not include intramuscular administration of the clostridial neurotoxin.

25. The clostridial neurotoxin for use according to any one of claims 1, 3-8 or 10-24 or the method according to any one of claims 2-7 or 9-24, wherein the patient is administered 100-500 U of the clostridial neurotoxin; preferably wherein the patient is administered 200 U of the clostridial neurotoxin.

26. The clostridial neurotoxin for use according to any one of claims 1, 3-8 or 10-25 or the method according to any one of claims 2-7 or 9-25, wherein the patient is administered a total dose of 1-3 ng of the clostridial neurotoxin.

27. The clostridial neurotoxin for use according to any one of claims 1, 3-8 or 10-26 or the method according to any one of claims 2-7 or 9-26, wherein the patient is administered 80-250 pg of the clostridial neurotoxin per kg (bodyweight).

28. The clostridial neurotoxin for use according to any one of claims 1, 3-8 or 10-27 or the method according to any one of claims 2-7 or 9-27, wherein the clostridial neurotoxin is administered at more than one administration site; preferably wherein the patient is administered 2.5-30 U of the clostridial neurotoxin per administration site; more preferably wherein the patient is administered 20 U of the clostridial neurotoxin per administration site.

29. The clostridial neurotoxin for use according to any one of claims 1, 3-8 or 10-28 or the method according to any one of claims 2-7 or 9-28, wherein the clostridial neurotoxin is administered at more than one administration site; preferably wherein the patient is administered 10-170 pg of the clostridial neurotoxin per administration site; more preferably 1-14 pg/kg bodyweight per administration site.


SEQUENCE LISTING (21 sequences)

SEQ ID NO: 1 - protein, 1296 aa, Clostridium botulinum
MPFVNKQFNYKDPVNGVDIAYIKIPNAGQMQPVKAFKIHNKIWVIPERDTFTNPEEGDLNPPPEAKQVPVSYYDSTYLSTDNEKDNYLKGVTKLFERIYSTDLGRMLLTSIVRGIPFWGGSTIDTELKVIDTNCINVIQPDGSYRSEELNLVIIGPSADIIQFECKSFGHEVLNLTRNGYGSTQYIRFSPDFTFGFEESLEVDTNPLLGAGKFATDPAVTLAHELIHAGHRLYGIAINPNRVFKVNTNAYYEMSGLEVSFEELRTFGGHDAKFIDSLQENEFRLYYYNKFKDIASTLNKAKSIVGTTASLQYMKNVFKEKYLLSEDTSGKFSVDKLKFDKLYKMLTEIYTEDNFVKFFKVLNRKTYLNFDKAVFKINIVPKVNYTIYDGFNLRNTNLAANFNGQNTEINNMNFTKLKNFTGLFEFYKLLCVRGIITSKTKSLDKGYNKALNDLCIKVNNWDLFFSPSEDNFTNDLNKGEEITSDTNIEAAEENISLDLIQQYYLTFNFDNEPENISIENLSSDIIGQLELMPNIERFPNGKKYELDKYTMFHYLRAQEFEHGKSRIALTNSVNEALLNPSRVYTFFSSDYVKKVNKATEAAMFLGWVEQLVYDFTDETSEVSTTDKIADITIIIPYIGPALNIGNMLYKDDFVGALIFSGAVILLEFIPEIAIPVLGTFALVSYIANKVLTVQTIDNALSKRNEKWDEVYKYIVTNWLAKVNTQIDLIRKKMKEALENQAEATKAIINYQYNQYTEEEKNNINFNIDDLSSKLNESINKAMININKFLNQCSVSYLMNSMIPYGVKRLEDFDASLKDALLKYIYDNRGTLIGQVDRLKDKVNNTLSTDIPFQLSKYVDNQRLLSTFTEYIKNIINTSILNLRYESNHLIDLSRYASKINIGSKVNFDPIDKNQIQLFNLESSKIEVILKNAIVYNSMYENFSTSFWIRIPKYFNSISLNNEYTIINCMENNSGWKVSLNYGEIIWTLQDTQEIKQRVVFKYSQMINISDYINRWIFVTITNNRLNNSKIYINGRLIDQKPISNLGNIHASNNIMFKLDGCRDTHRYIWIKYFNLFDKELNEKEIKDLYDNQSNSGILKDFWGDYLQYDKPYYMLNLYDPNKYVDVNNVGIRGYMYLKGPRGSVMTTNIYLNSSLYRGTKFIIKKYASGNKDNIVRNNDRVYINVVVKNKEYRLATNASQAGVEKILSALEIPDVGNLSQVVVMKSKNDQGITNKCKMNLQDNNGNDIGFIGFHQFNNIAKLVASNWYNRQIERSSRTLGCSWEFIPVDDGWGERPL

SEQ ID NO: 2 - protein, 1291 aa, Clostridium botulinum
MPVTINNFNYNDPIDNNNIIMMEPPFARGTGRYYKAFKITDRIWIIPERYTFGYKPEDFNKSSGIFNRDVCEYYDPDYLNTNDKKNIFLQTMIKLFNRIKSKPLGEKLLEMIINGIPYLGDRRVPLEEFNTNIASVTVNKLISNPGEVERKKGIFANLIIFGPGPVLNENETIDIGIQNHFASREGFGGIMQMKFCPEYVSVFNNVQENKGASIFNRRGYFSDPALILMHELIHVLHGLYGIKVDDLPIVPNEKKFFMQSTDAIQAEELYTFGGQDPSIITPSTDKSIYDKVLQNFRGIVDRLNKVLVCISDPNININIYKNKFKDKYKFVEDSEGKYSIDVESFDKLYKSLMFGFTETNIAENYKIKTRASYFSDSLPPVKIKNLLDNEIYTIEEGFNISDKDMEKEYRGQNKAINKQAYEEISKEHLAVYKIQMCKSVKAPGICIDVDNEDLFFIADKNSFSDDLSKNERIEYNTQSNYIENDFPINELILDTDLISKIELPSENTESLTDFNVDVPVYEKQPAIKKIFTDENTIFQYLYSQTFPLDIRDISLTSSFDDALLFSNKVYSFFSMDYIKTANKVVEAGLFAGWVKQIVNDFVIEANKSNTMDKIADISLIVPYIGLALNVGNETAKGNFENAFEIAGASILLEFIPELLIPVVGAFLLESYIDNKNKIIKTIDNALTKRNEKWSDMYGLIVAQWLSTVNTQFYTIKEGMYKALNYQAQALEEIIKYRYNIYSEKEKSNINIDFNDINSKLNEGINQAIDNINNFINGCSVSYLMKKMIPLAVEKLLDFDNTLKKNLLNYIDENKLYLIGSAEYEKSKVNKYLKTIMPFDLSIYTNDTILIEMFNKYNSEILNNIILNLRYKDNNLIDLSGYGAKVEVYDGVELNDKNQFKLTSSANSKIRVTQNQNIIFNSVFLDFSVSFWIRIPKYKNDGIQNYIHNEYTIINCMKNNSGWKISIRGNRIIWTLIDINGKTKSVFFEYNIREDISEYINRWFFVTITNNLNNAKIYINGKLESNTDIKDIREVIANGEIIFKLDGDIDRTQFIWMKYFSIFNTELSQSNIEERYKIQSYSEYLKDFWGNPLMYNKEYYMFNAGNKNSYIKLKKDSPVGEILTRSKYNQNSKYINYRDLYIGEKFIIRRKSNSQSINDDIVRKEDYIYLDFFNLNQEWRVYTYKYFKKEEEKLFLAPISDSDEFYNTIQIKEYDEQPTYSCQLLFKKDEESTDEIGLIGIHRFYESGIVFEEYKDYFCISKWYLKEVKRKPYNLKLGCNWQFIPKDEGWTE

SEQ ID NO: 3 - protein, 1291 aa, Clostridium botulinum
MPITINNFNYSDPVDNKNILYLDTHLNTLANEPEKAFRITGNIWVIPDRFSRNSNPNLNKPPRVTSPKSGYYDPNYLSTDSDKDPFLKEIIKLFKRINSREIGEELIYRLSTDIPFPGNNNTPINTFDFDVDFNSVDVKTRQGNNWVKTGSINPSVIITGPRENIIDPETSTFKLTNNTFAAQEGFGALSIISISPRFMLTYSNATNDVGEGRFSKSEFCMDPILILMHELNHAMHNLYGIAIPNDQTISSVTSNIFYSQYNVKLEYAEIYAFGGPTIDLIPKSARKYFEEKALDYYRSIAKRLNSITTANPSSFNKYIGEYKQKLIRKYRFVVESSGEVTVNRNKFVELYNELTQIFTEFNYAKIYNVQNRKIYLSNVYTPVTANILDDNVYDIQNGFNIPKSNLNVLFMGQNLSRNPALRKVNPENMLYLFTKFCHKAIDGRSLYNKTLDCRELLVKNTDLPFIGDISDVKTDIFLRKDINEETEVIYYPDNVSVDQVILSKNTSEHGQLDLLYPSIDSESEILPGENQVFYDNRTQNVDYLNSYYYLESQKLSDNVEDFTFTRSIEEALDNSAKVYTYFPTLANKVNAGVQGGLFLMWANDVVEDFTTNILRKDTLDKISDVSAIIPYIGPALNISNSVRRGNFTEAFAVTGVTILLEAFPEFTIPALGAFVIYSKVQERNEIIKTIDNCLEQRIKRWKDSYEWMMGTWLSRIITQFNNISYQMYDSLNYQAGAIKAKIDLEYKKYSGSDKENIKSQVENLKNSLDVKISEAMNNINKFIRECSVTYLFKNMLPKVIDELNEFDRNTKAKLINLIDSHNIILVGEVDKLKAKVNNSFQNTIPFNIFSYTNNSLLKDIINEYFNNINDSKILSLQNRKNTLVDTSGYNAEVSEEGDVQLNPIFPFDFKLGSSGEDRGKVIVTQNENIVYNSMYESFSISFWIRINKWVSNLPGYTIIDSVKNNSGWSIGIISNFLVFTLKQNEDSEQSINFSYDISNNAPGYNKWFFVTVTNNMMGNMKIYINGKLIDTIKVKELTGINFSKTITFEINKIPDTGLITSDSDNINMWIRDFYIFAKELDGKDINILFNSLQYTNVVKDYWGNDLRYNKEYYMVNIDYLNRYMYANSRQIVFNTRRNNNDFNEGYKIIIKRIRGNTNDTRVRGGDILYFDMTINNKAYNLFMKNETMYADNHSTEDIYAIGLREQTKDINDNIIFQIQPMNNTYYYASQIFKSNFNGENISGICSIGTYRFRLGGDWYRHNYLVPTVKQGNYASLLESTSTHWGFVPVSE

SEQ ID NO: 4 - protein, 1276 aa, Clostridium botulinum
MTWPVKDFNYSDPVNDNDILYLRIPQNKLITTPVKAFMITQNIWVIPERFSSDTNPSLSKPPRPTSKYQSYYDPSYLSTDEQKDTFLKGIIKLFKRINERDIGKKLINYLVVGSPFMGDSSTPEDTFDFTRHTTNIAVEKFENGSWKVTNIITPSVLIFGPLPNILDYTASLTLQGQQSNPSFEGFGTLSILKVAPEFLLTFSDVTSNQSSAVLGKSIFCMDPVIALMHELTHSLHQLYGINIPSDKRIRPQVSEGFFSQDGPNVQFEELYTFGGLDVEIIPQIERSQLREKALGHYKDIAKRLNNINKTIPSSWISNIDKYKKIFSEKYNFDKDNTGNFVVNIDKFNSLYSDLTNVMSEVVYSSQYNVKNRTHYFSRHYLPVFANILDDNIYTIRDGFNLTNKGFNIENSGQNIERNPALQKLSSESVVDLFTKVCLRLTKNSRDDSTCIKVKNNRLPYVADKDSISQEIFENKIITDETNVQNYSDKFSLDESILDGQVPINPEIVDPLLPNVNMEPLNLPGEEIVFYDDITKYVDYLNSYYYLESQKLSNNVENITLTTSVEEALGYSNKIYTFLPSLAEKVNKGVQAGLFLNWANEVVEDFTTNIMKKDTLDKISDVSVIIPYIGPALNIGNSALRGNFNQAFATAGVAFLLEGFPEFTIPALGVFTFYSSIQEREKIIKTIENCLEQRVKRWKDSYQWMVSNWLSRITTQFNHINYQMYDSLSYQADAIKAKIDLEYKKYSGSDKENIKSQVENLKNSLDVKISEAMNNINKFIRECSVTYLFKNMLPKVIDELNKFDLRTKTELINLIDSHNIILVGEVDRLKAKVNESFENTMPFNIFSYTNNSLLKDIINEYFNSINDSKILSLQNKKNALVDTSGYNAEVRVGDNVQLNTIYTNDFKLSSSGDKIIVNLNNNILYSAIYENSSVSFWIKISKDLTNSHNEYTIINSIEQNSGWKLCIRNGNIEWILQDVNRKYKSLIFDYSESLSHTGYTNKWFFVTITNNIMGYMKLYINGELKQSQKIEDLDEVKLDKTIVFGIDENIDENQMLWIRDFNIFSKELSNEDINIVYEGQILRNVIKDYWGNPLKFDTEYYIINDNYIDRYIAPESNVLVLVQYPDRSKLYTGNPITIKSVSDKNPYSRILNGDNIILHMLYNSRKYMIIRDTDTIYATQGGECSQNCVYALKLQSNLGNYGIGIFSIKNIVSKNKYCSQIFSSFRENTMLLADIYKPWRFSFKNAYTPVAVTNYETKLLSTSSFWKFISRDPGWVE

SEQ ID NO: 5 - protein, 1252 aa, Clostridium botulinum
MPKINSFNYNDPVNDRTILYIKPGGCQEFYKSFNIMKNIWIIPERNVIGTTPQDFHPPTSLKNGDSSYYDPNYLQSDEEKDRFLKIVTKIFNRINNNLSGGILLEELSKANPYLGNDNTPDNQFHIGDASAVEIKFSNGSQDILLPNVIIMGAEPDLFETNSSNISLRNNYMPSNHGFGSIAIVTFSPEYSFRFNDNSMNEFIQDPALTLMHELIHSLHGLYGAKGITTKYTITQKQNPLITNIRGTNIEEFLTFGGTDLNIITSAQSNDIYTNLLADYKKIASKLSKVQVSNPLLNPYKDVFEAKYGLDKDASGIYSVNINKFNDIFKKLYSFTEFDLATKFQVKCRQTYIGQYKYFKLSNLLNDSIYNISEGYNINNLKVNFRGQNANLNPRIITPITGRGLVKKIIRFCKNIVSVKGIRKSICIEINNGELFFVASENSYNDDNINTPKEIDDTVTSNNNYENDLDQVILNFNSESAPGLSDEKLNLTIQNDAYIPKYDSNGTSDIEQHDVNELNVFFYLDAQKVPEGENNVNLTSSIDTALLEQPKIYTFFSSEFINNVNKPVQAALFVSWIQQVLVDFTTEANQKSTVDKIADISIVVPYIGLALNIGNEAQKGNFKDALELLGAGILLEFEPELLIPTILVFTIKSFLGSSDNKNKVIKAINNALKERDEKWKEVYSFIVSNWMTKINTQFNKRKEQMYQALQNQVNAIKTIIESKYNSYTLEEKNELTNKYDIKQIENELNQKVSIAMNNIDRFLTESSISYLMKLINEVKINKLREYDENVKTYLLNYIIQHGSILGESQQELNSMVTDTLNNSIPFKLSSYTDDKILISYFNKFFKRIKSSSVLNMRYKNDKYVDTSGYDSNININGDVYKYPTNKNQFGIYNDKLSEVNISQNDYIIYDNKYKNFSISFWVRIPNYDNKIVNVNNEYTIINCMRDNNSGWKVSLNHNEIIWTLQDNAGINQKLAFNYGNANGISDYINKWIFVTITNDRLGDSKLYINGNLIDQKSILNLGNIHVSDNILFKIVNCSYTRYIGIRYFNIFDKELDETEIQTLYSNEPNTNILKDFWGNYLLYDKEYYLLNVLKPNNFIDRRKDSTLSINNIRSTILLANRLYSGIKVKIQRVNNSSTNDNLVRKNDQVYINFVASKTHLFPLYADTATTNKEKTIKISSSGNRFNQVVVMNSVGNNCTMNFKNNNGNNIGLLGFKADTVVASTWYYTHMRDHTNSNGCFWNFISEEHGWQEK

SEQ ID NO: 6 - protein, 1278 aa, Clostridium botulinum
MPVVINSFNYNDPVNDDTILYMQIPYEEKSKKYYKAFEIMRNVWIIPERNTIGTDPSDFDPPASLENGSSAYYDPNYLTTDAEKDRYLKTTIKLFKRINSNPAGEVLLQEISYAKPYLGNEHTPINEFHPVTRTTSVNIKSSTNVKSSIILNLLVLGAGPDIFENSSYPVRKLMDSGGVYDPSNDGFGSINIVTFSPEYEYTFNDISGGYNSSTESFIADPAISLAHELIHALHGLYGARGVTYKETIKVKQAPLMIAEKPIRLEEFLTFGGQDLNIITSAMKEKIYNNLLANYEKIATRLSRVNSAPPEYDINEYKDYFQWKYGLDKNADGSYTVNENKFNEIYKKLYSFTEIDLANKFKVKCRNTYFIKYGFLKVPNLLDDDIYTVSEGFNIGNLAVNNRGQNIKLNPKIIDSIPDKGLVEKIVKFCKSVIPRKGTKAPPRLCIRVNNRELFFVASESSYNENDINTPKEIDDTTNLNNNYRNNLDEVILDYNSETIPQISNQTLNTLVQDDSYVPRYDSNGTSEIEEHNVVDLNVFFYLHAQKVPEGETNISLTSSIDTALSEESQVYTFFSSEFINTINKPVHAALFISWINQVIRDFTTEATQKSTFDKIADISLVVPYVGLALNIGNEVQKENFKEAFELLGAGILLEFVPELLIPTILVFTIKSFIGSSENKNKIIKAINNSLMERETKWKEIYSWIVSNWLTRINTQFNKRKEQMYQALQNQVDAIKTVIEYKYNNYTSDERNRLESEYNINNIREELNKKVSLAMENIERFITESSIFYLMKLINEAKVSKLREYDEGVKEYLLDYISEHRSILGNSVQELNDLVTSTLNNSIPFELSSYTNDKILILYFNKLYKKIKDNSILDMRYENNKFIDISGYGSNISINGDVYIYSTNRNQFGIYSSKPSEVNIAQNNDIIYNGRYQNFSISFWVRIPKYFNKVNLNNEYTIIDCIRNNNSGWKISLNYNKIIWTLQDTAGNNQKLVFNYTQMISISDYINKWIFVTITNNRLGNSRIYINGNLIDEKSISNLGDIHVSDNILFKIVGCNDTRYVGIRYFKVFDTELGKTEIETLYSDEPDPSILKDFWGNYLLYNKRYYLLNLLRTDKSITQNSNFLNINQQRGVYQKPNIFSNTRLYTGVEVIIRKNGSTDISNTDNFVRKNDLAYINVVDRDVEYRLYADISIAKPEKIIKLIRTSNSNNSLGQIIVMDSIGNNCTMNFQNNNGGNIGLLGFHSNNLVASSWYYNNIRKNTSSNGCFWSFISKEHGWQEN

SEQ ID NO: 7 - protein, 1297 aa, Clostridium botulinum
MPVNIKNFNYNDPINNDDIIMMEPFNDPGPGTYYKAFRIIDRIWIVPERFTYGFQPDQFNASTGVFSKDVYEYYDPTYLKTDAEKDKFLKTMIKLFNRINSKPSGQRLLDMIVDAIPYLGNASTPPDKFAANVANVSINKKIIQPGAEDQIKGLMTNLIIFGPGPVLSDNFTDSMIMNGHSPISEGFGARMMIRFCPSCLNVFNNVQENKDTSIFSRRAYFADPALTLMHELIHVLHGLYGIKISNLPITPNTKEFFMQHSDPVQAEELYTFGGHDPSVISPSTDMNIYNKALQNFQDIANRLNIVSSAQGSGIDISLYKQIYKNKYDFVEDPNGKYSVDKDKFDKLYKALMFGFTETNLAGEYGIKTRYSYFSEYLPPIKTEKLLDNTIYTQNEGFNIASKNLKTEFNGQNKAVNKEAYEEISLEHLVIYRIAMCKPVMYKNTGKSEQCIIVNNEDLFFIANKDSFSKDLAKAETIAYNTQNNTIENNFSIDQLILDNDLSSGIDLPNENTEPFTNFDDIDIPVYIKQSALKKIFVDGDSLFEYLHAQTFPSNIENLQLTNSLNDALRNNNKVYTFFSTNLVEKANTVVGASLFVNWVKGVIDDFTSESTQKSTIDKVSDVSIIIPYIGPALNVGNETAKENFKNAFEIGGAAILMEFIPELIVPIVGFFTLESYVGNKGHIIMTISNALKKRDQKWTDMYGLIVSQWLSTVNTQFYTIKERMYNALNNQSQAIEKIIEDQYNRYSEEDKMNINIDFNDIDFKLNQSINLAINNIDDFINQCSISYLMNRMIPLAVKKLKDFDDNLKRDLLEYIDTNELYLLDEVNILKSKVNRHLKDSIPFDLSLYTKDTILIQVFNNYISNISSNAILSLSYRGGRLIDSSGYGATMNVGSDVIFNDIGNGQFKLNNSENSNITAHQSKFVVYDSMFDNFSINFWVRTPKYNNNDIQTYLQNEYTIISCIKNDSGWKVSIKGNRIIWTLIDVNAKSKSIFFEYSIKDNISDYINKWFSITITNDRLGNANIYINGSLKKSEKILNLDRINSSNDIDFKLINCTDTTKFVWIKDFNIFGRELNATEVSSLYWIQSSTNTLKDFWGNPLRYDTQYYLFNQGMQNIYIKYFSKASMGETAPRTNFNNAAINYQNLYLGLRFIIKKASNSRNINNDNIVREGDYIYLNIDNISDESYRVYVLVNSKEIQTQLFLAPINDDPTFYDVLQIKKYYEKTTYNCQILCEKDTKTFGLFGIGKFVKDYGYVWDTYDNYFCISQWYLRRISENINKLRLGCNWQFIPVDEGWTE

SEQ ID NO: 8 - protein, 1285 aa, Clostridium botulinum
MTWPVKDFNYSDPVNDNDILYLRIPQNKLITTPVKAFMITQNIWVIPERFSSDTNPSLSKPPRPTSKYQSYYDPSYLSTDEQKDTFLKGIIKLFKRINERDIGKKLINYLVVGSPFMGDSSTPEDTFDFTRHTTNIAVEKFENGSWKVTNIITPSVLIFGPLPNILDYTASLTLQGQQSNPSFEGFGTLSILKVAPEFLLTFSDVTSNQSSAVLGKSIFCMDPVIALMHELTHSLHQLYGINIPSDKRIRPQVSEGFFSQDGPNVQFEELYTFGGSDVEIIPQIERLQLREKALGHYKDIAKRLNNINKTIPSSWSSNIDKYKKIFSEKYNFDKDNTGNFVVNIDKFNSLYSDLTNVMSEVVYSSQYNVKNRTHYFSKHYLPVFANILDDNIYTIINGFNLTTKGFNIENSGQNIERNPALQKLSSESVVDLFTKVCLRLTRNSRDDSTCIQVKNNTLPYVADKDSISQEIFESQIITDETNVENYSDNFSLDESILDAKVPTNPEAVDPLLPNVNMEPLNVPGEEEVFYDDITKDVDYLNSYYYLEAQKLSNNVENITLTTSVEEALGYSNKIYTFLPSLAEKVNKGVQAGLFLNWANEVVEDFTTNIMKKDTLDKISDVSAIIPYIGPALNIGNSALRGNFKQAFATAGVAFLLEGFPEFTIPALGVFTFYSSIQEREKIIKTIENCLEQRVKRWKDSYQWMVSNWLSRITTQFNHISYQMYDSLSYQADAIKAKIDLEYKKYSGSDKENIKSQVENLKNSLDVKISEAMNNINKFIRECSVTYLFKNMLPKVIDELNKFDLKTKTELINLIDSHNIILVGEVDRLKAKVNESFENTIPFNIFSYTNNSLLKDMINEYFNSINDSKILSLQNKKNTLMDTSGYNAEVRVEGNVQLNPIFPFDFKLGSSGDDRGKVIVTQNENIVYNAMYESFSISFWIRINKWVSNLPGYTIIDSVKNNSGWSIGIISNFLVFTLKQNENSEQDINFSYDISKNAAGYNKWFFVTITTNMMGNMMIYINGKLIDTIKVKELTGINFSKTITFQMNKIPNTGLITSDSDNINMWIRDFYIFAKELDDKDINILFNSLQYTNVVKDYWGNDLRYDKEYYMINVNYMNRYMSKKGNGIVFNTRKNNNDFNEGYKIIIKRIIGNTNDTRVRGENVLYFNTTIDNKQYSLGMYKPSRNLGTDLVPLGALDQPMDEIRKYGSFIIQPCNTFDYYASQLFLSSNATTNRIGILSIGSYSFKLGDDYWFNHEYLIPVIKIEHYASLLESTSTHWVFVPASE

SEQ ID NO: 9 - protein, 1268 aa, Clostridium botulinum
MPVNINNFNYNDPINNTTILYMKMPYYEDSNKYYKAFEIMDNVWIIPERNIIGKKPSDFYPPISLDSGSSAYYDPNYLTTDAEKDRFLKTVIKLFNRINSNPAGQVLLEEIKNGKPYLGNDHTAVNEFCANNRSTSVEIKESKGTTDSMLLNLVILGPGPNILECSTFPVRIFPNNIAYDPSEKGFGSIQLMSFSTEYEYAFNDNTDLFIADPAISLAHELIHVLHGLYGAKGVTNKKVIEVDQGALMAAEKDIKIEEFITFGGQDLNIITNSTNQKIYDNLLSNYTAIASRLSQVNINNSALNTTYYKNFFQWKYGLDQDSNGNYTVNISKFNAIYKKLFSFTECDLAQKFQVKNRSNYLFHFKPFRLLDLLDDNIYSISEGFNIGSLRVNNNGQNINLNSRIVGPIPDNGLVERFVGLCKSIVSKKGTKNSLCIKVNNRDLFFVASESSYNENGINSPKEIDDTTITNNNYKKNLDEVILDYNSDAIPNLSSRLLNTTAQNDSYVPKYDSNGTSEIKEYTVDKLNVFFYLYAQKAPEGESAISLTSSVNTALLDASKVYTFFSSDFINTVNKPVQAALFISWIQQVINDFTTEATQKSTIDKIADISLVVPYVGLALNIGNEVQKGNFKEAIELLGAGILLEFVPELLIPTILVFTIKSFINSDDSKNKIIKAINNALRERELKWKEVYSWIVSNWLTRINTQFNKRKEQMYQALQNQVDGIKKIIEYKYNNYTLDEKNRLKAEYNIYSIKEELNKKVSLAMQNIDRFLTESSISYLMKLINEAKINKLSEYDKRVNQYLLNYILENSSTLGTSSVQELNNLVSNTLNNSIPFELSEYTNDKILISYFNRFYKRIIDSSILNMKYENNRFIDSSGYGSNISINGDIYIYSTNRNQFGIYSSRLSEVNITQNNTIIYNSRYQNFSVSFWVRIPKYNNLKNLNNEYTIINCMRNNNSGWKISLNYNNIIWTLQDTTGNNQKLVFNYTQMIDISDYINKWTFVTITNNRLGHSKLYINGNLTDQKSILNLGNIHVDDNILFKIVGCNDTRYVGIRYFKIFNMELDKTEIETLYHSEPDSTILKDFWGNYLLYNKKYYLLNLLKPNMSVTKNSDILNINRQRGIYSKTNIFSNARLYTGVEVIIRKVGSTDTSNTDNFVRKNDTVYINVVDGNSEYQLYADVSTSAVEKTIKLRRISNSNYNSNQMIIMDSIGDNCTMNFKTNNGNDIGLLGFHLNNLVASSWYYKNIRNNTRNNGCFWSFISKEHGWQE

SEQ ID NO: 10 - protein, 1304 aa, Artificial Sequence
  BoNT/ABMY, amino acid sequence
MPFVNKQFNYKDPVNGVDIAYIKIPNAGQMQPVKAFKIHNKIWVIPERDTFTNPEEGDLNPPPEAKQVPVSYYDSTYLSTDNEKDNYLKGVTKLFERIYSTDLGRMLLTSIVRGIPFWGGSTIDTELKVIDTNCINVIQPDGSYRSEELNLVIIGPSADIIQFECKSFGHEVLNLTRNGYGSTQYIRFSPDFTFGFEESLEVDTNPLLGAGKFATDPAVTLAHELIHAGHRLYGIAINPNRVFKVNTNAYYEMSGLEVSFEELRTFGGHDAKFIDSLQENEFRLYYYNKFKDIASTLNKAKSIVGTTASLQYMKNVFKEKYLLSEDTSGKFSVDKLKFDKLYKMLTEIYTEDNFVKFFKVLNRKTYLNFDKAVFKINIVPKVNYTIYDGFNLRNTNLAANFNGQNTEINNMNFTKLKNFTGLFEFYKLLCVRGIITSKTKSLDKGYNKALNDLCIKVNNWDLFFSPSEDNFTNDLNKGEEITSDTNIEAAEENISLDLIQQYYLTFNFDNEPENISIENLSSDIIGQLELMPNIERFPNGKKYELDKYTMFHYLRAQEFEHGKSRIALTNSVNEALLNPSRVYTFFSSDYVKKVNKATEAAMFLGWVEQLVYDFTDETSEVSTTDKIADITIIIPYIGPALNIGNMLYKDDFVGALIFSGAVILLEFIPEIAIPVLGTFALVSYIANKVLTVQTIDNALSKRNEKWDEVYKYIVTNWLAKVNTQIDLIRKKMKEALENQAEATKAIINYQYNQYTEEEKNNINFNIDDLSSKLNESINKAMININKFLNQCSVSYLMNSMIPYGVKRLEDFDASLKDALLKYIYDNRGTLIGQVDRLKDKVNNTLSTDIPFQLSKYVDNQRLLSTFTEYIKNILNNIILNLRYKDNNLIDLSGYGAKVEVYDGVELNDKNQFKLTSSANSKIRVTQNQNIIFNSVFLDFSVSFWIRIPKYKNDGIQNYIHNEYTIINCMKNNSGWKISIRGNRIIWTLIDINGKTKSVFFEYNIREDISEYINRWFFVTITNNLNNAKIYINGKLESNTDIKDIREVIANGEIIFKLDGDIDRTQFIWMKYFSIFNTELSQSNIEERYKIQSYSEYLKDFWGNPLMYNKEYYMFNAGNKNSYIKLKKDSPVGEILTRSKYNQNSKYINYRDLYIGEKFIIRRKSNSQSINDDIVRKEDYIYLDFFNLNQEWRVYTYKYFKKEEMKLFLAPIYDSDEFYNTIQIKEYDEQPTYSCQLLFKKDEESTDEIGLIGIHRFYESGIVFEEYKDYFCISKWYLKEVKRKPYNLKLGCNWQFIPKDEGWTE

SEQ ID NO: 11 - protein, 1306 aa, Clostridium botulinum
MKLEINKFNYNDPIDGINVITMRPPRHSDKINKGKGPFKAFQVIKNIWIVPERYNFTNNTNDLNIPSEPIMEADAIYNPNYLNTPSEKDEFLQGVIKVLERIKSKPEGEKLLELISSSIPLPLVSNGALTLSDNETIAYQENNNIVSNLQANLVIYGPGPDIANNATYGLYSTPISNGEGTLSEVSFSPFYLKPFDESYGNYRSLVNIVNKFVKREFAPDPASTLMHELVHVTHNLYGISNRNFYYNFDTGKIETSRQQNSLIFEELLTFGGIDSKAISSLIIKKIIETAKNNYTTLISERLNTVTVENDLLKYIKNKIPVQGRLGNFKLDTAEFEKKLNTILFVLNESNLAQRFSILVRKHYLKERPIDPIYVNILDDNSYSTLEGFNISSQGSNDFQGQLLESSYFEKIESNALRAFIKICPRNGLLYNAIYRNSKNYLNNIDLEDKKTTSKTNVSYPCSLLNGCIEVENKDLFLISNKDSLNDINLSEEKIKPETTVFFKDKLPPQDITLSNYDFTEANSIPSISQQNILERNEELYEPIRNSLFEIKTIYVDKLTTFHFLEAQNIDESIDSSKIRVELTDSVDEALSNPNKVYSPFKNMSNTINSIETGITSTYIFYQWLRSIVKDFSDETGKIDVIDKSSDTLAIVPYIGPLLNIGNDIRHGDFVGAIELAGITALLEYVPEFTIPILVGLEVIGGELAREQVEAIVNNALDKRDQKWAEVYNITKAQWWGTIHLQINTRLAHTYKALSRQANAIKMNMEFQLANYKGNIDDKAKIKNAISETEILLNKSVEQAMKNTEKFMIKLSNSYLTKEMIPKVQDNLKNFDLETKKTLDKFIKEKEDILGTNLSSSLRRKVSIRLNKNIAFDINDIPFSEFDDLINQYKNEIEDYEVLNLGAEDGKIKDLSGTTSDINIGSDIELADGRENKAIKIKGSENSTIKIAMNKYLRFSATDNFSISFWIKHPKPTNLLNNGIEYTLVENFNQRGWKISIQDSKLIWYLRDHNNSIKIVTPDYIAFNGWNLITITNNRSKGSIVYVNGSKIEEKDISSIWNTEVDDPIIFRLKNNRDTQAFTLLDQFSIYRKELNQNEVVKLYNYYFNSNYIRDIWGNPLQYNKKYYLQTQDKPGKGLIREYWSSFGYDYVILSDSKTITFPNNIRYGALYNGSKVLIKNSKKLDGLVRNKDFIQLEIDGYNMGISADRFNEDTNYIGTTYGTTHDLTTDFEIIQRQEKYRNYCQLKTPYNIFHKSGLMSTETSKPTFHDYRDWVYSSAWYFQNYENLNLRKHTKTNWYFIPKDEGWDED

SEQ ID NO: 12 - DNA, 3888 bp, Clostridium botulinum
atgccattcgtcaacaagcaattcaactacaaagacccagtcaacggcgtcgacatcgcatacatcaagattccgaacgccggtcaaatgcagccggttaaggcttttaagatccacaacaagatttgggttatcccggagcgtgacaccttcacgaacccggaagaaggcgatctgaacccgccaccggaagcgaagcaagtccctgtcagctactacgattcgacgtacctgagcacggataacgaaaaagataactacctgaaaggtgtgaccaagctgttcgaacgtatctacagcacggatctgggtcgcatgctgctgactagcattgttcgcggtatcccgttctggggtggtagcacgattgacaccgaactgaaggttatcgacactaactgcattaacgttattcaaccggatggtagctatcgtagcgaagagctgaatctggtcatcattggcccgagcgcagacattatccaattcgagtgcaagagctttggtcacgaggttctgaatctgacccgcaatggctatggtagcacccagtacattcgtttttcgccggattttaccttcggctttgaagagagcctggaggttgataccaatccgttgctgggtgcgggcaaattcgctaccgatccggctgtcacgctggcccatgaactgatccacgcaggccaccgcctgtacggcattgccatcaacccaaaccgtgtgttcaaggttaatacgaatgcatactacgagatgagcggcctggaagtcagcttcgaagaactgcgcaccttcggtggccatgacgctaaattcattgacagcttgcaagagaatgagttccgtctgtactactataacaaattcaaagacattgcaagcacgttgaacaaggccaaaagcatcgttggtactaccgcgtcgttgcagtatatgaagaatgtgtttaaagagaagtacctgctgtccgaggatacctccggcaagtttagcgttgataagctgaagtttgacaaactgtacaagatgctgaccgagatttacaccgaggacaactttgtgaaattcttcaaagtgttgaatcgtaaaacctatctgaattttgacaaagcggttttcaagattaacatcgtgccgaaggtgaactacaccatctatgacggttttaacctgcgtaacaccaacctggcggcgaactttaacggtcagaatacggaaatcaacaacatgaatttcacgaagttgaagaacttcacgggtctgttcgagttctataagctgctgtgcgtgcgcggtatcatcaccagcaaaaccaaaagcctggacaaaggctacaacaaggcgctgaatgacctgtgcattaaggtaaacaattgggatctgttcttttcgccatccgaagataattttaccaacgacctgaacaagggtgaagaaatcaccagcgatacgaatattgaagcagcggaagagaatatcagcctggatctgatccagcagtactatctgacctttaacttcgacaatgaaccggagaacattagcattgagaatctgagcagcgacattatcggtcagctggaactgatgccgaatatcgaacgtttcccgaacggcaaaaagtacgagctggacaagtacactatgttccattacctgcgtgcacaggagtttgaacacggtaaaagccgtatcgcgctgaccaacagcgttaacgaggccctgctgaacccgagccgtgtctataccttcttcagcagcgactatgttaagaaagtgaacaaagccactgaggccgcgatgttcctgggctgggtggaacagctggtatatgacttcacggacgagacgagcgaagtgagcactaccgacaaaattgctgatattaccatcattatcccgtatattggtccggcactgaacattggcaacatgctgtacaaagacgattttgtgggtgccctgatcttctccggtgccgtgattctgctggagttcattccggagattgcgatcccggtgttgggtaccttcgcgctggtgtcctacatcgcgaataaggttctgacggttcagaccatcgataacgcgctgtcgaaacgtaatgaaaaatgggacgaggtttacaaatacattgttacgaattggctggcgaaagtcaatacccagatcgacctgatccgtaagaaaatgaaagaggcgctggagaatcaggcggaggccaccaaagcaattatcaactaccaatacaaccagtacacggaagaagagaagaataacattaacttcaatatcgatgatttgagcagcaagctgaatgaatctatcaacaaagcgatgatcaatatcaacaagtttttgaatcagtgtagcgtttcgtacctgatgaatagcatgattccgtatggcgtcaaacgtctggaggacttcgacgccagcctgaaagatgcgttgctgaaatacatttacgacaatcgtggtacgctgattggccaagttgaccgcttgaaagacaaagttaacaataccctgagcaccgacatcccatttcaactgagcaagtatgttgataatcaacgtctgttgagcactttcaccgagtatatcaaaaacatcatcaatactagcattctgaacctgcgttacgagagcaatcatctgattgatctgagccgttatgcaagcaagatcaacatcggtagcaaggtcaattttgacccgatcgataagaaccagatccagctgtttaatctggaatcgagcaaaattgaggttatcctgaaaaacgccattgtctacaactccatgtacgagaatttctccaccagcttctggattcgcatcccgaaatacttcaacagcattagcctgaacaacgagtatactatcatcaactgtatggagaacaacagcggttggaaggtgtctctgaactatggtgagatcatttggaccttgcaggacacccaagagatcaagcagcgcgtcgtgttcaagtactctcaaatgatcaacatttccgattacattaatcgttggatcttcgtgaccattacgaataaccgtctgaataacagcaagatttacatcaatggtcgcttgatcgatcagaaaccgattagcaacctgggtaatatccacgcaagcaacaacattatgttcaaattggacggttgccgcgatacccatcgttatatctggatcaagtatttcaacctgtttgataaagaactgaatgagaaggagatcaaagatttgtatgacaaccaatctaacagcggcattttgaaggacttctggggcgattatctgcaatacgataagccgtactatatgctgaacctgtatgatccgaacaaatatgtggatgtcaataatgtgggtattcgtggttacatgtatttgaagggtccgcgtggcagcgttatgacgaccaacatttacctgaactctagcctgtaccgtggtacgaaattcatcattaagaaatatgccagcggcaacaaagataacattgtgcgtaataacgatcgtgtctacatcaacgtggtcgtgaagaataaagagtaccgtctggcgaccaacgcttcgcaggcgggtgttgagaaaattctgagcgcgttggagatccctgatgtcggtaatctgagccaagtcgtggttatgaagagcaagaacgaccagggtatcactaacaagtgcaagatgaacctgcaagacaacaatggtaacgacatcggctttattggtttccaccagttcaacaatattgctaaactggtagcgagcaattggtacaatcgtcagattgagcgcagcagccgtactttgggctgtagctgggagtttatcccggtcgatgatggttggggcgaacgtccgctg

SEQ ID NO: 13 - protein, 1296 aa, Clostridium botulinum; identical to SEQ ID NO: 1

SEQ ID NO: 14 - DNA, 3888 bp, Artificial Sequence
  Nucleotide Sequence of Modified BoNT/A “Cat-A”
atgccattcgtcaacaagcaattcaactacaaagacccagtcaacggcgtcgacatcgcatacatcaagattccgaacgccggtcaaatgcagccggttaaggcttttaagatccacaacaagatttgggttatcccggagcgtgacaccttcacgaacccggaagaaggcgatctgaacccgccaccggaagcgaagcaagtccctgtcagctactacgattcgacgtacctgagcacggataacgaaaaagataactacctgaaaggtgtgaccaagctgttcgaacgtatctacagcacggatctgggtcgcatgctgctgactagcattgttcgcggtatcccgttctggggtggtagcacgattgacaccgaactgaaggttatcgacactaactgcattaacgttattcaaccggatggtagctatcgtagcgaagagctgaatctggtcatcattggcccgagcgcagacattatccaattcgagtgcaagagctttggtcacgaggttctgaatctgacccgcaatggctatggtagcacccagtacattcgtttttcgccggattttaccttcggctttgaagagagcctggaggttgataccaatccgttgctgggtgcgggcaaattcgctaccgatccggctgtcacgctggcccatgaactgatccacgcaggccaccgcctgtacggcattgccatcaacccaaaccgtgtgttcaaggttaatacgaatgcatactacgagatgagcggcctggaagtcagcttcgaagaactgcgcaccttcggtggccatgacgctaaattcattgacagcttgcaagagaatgagttccgtctgtactactataacaaattcaaagacattgcaagcacgttgaacaaggccaaaagcatcgttggtactaccgcgtcgttgcagtatatgaagaatgtgtttaaagagaagtacctgctgtccgaggatacctccggcaagtttagcgttgataagctgaagtttgacaaactgtacaagatgctgaccgagatttacaccgaggacaactttgtgaaattcttcaaagtgttgaatcgtaaaacctatctgaattttgacaaagcggttttcaagattaacatcgtgccgaaggtgaactacaccatctatgacggttttaacctgcgtaacaccaacctggcggcgaactttaacggtcagaatacggaaatcaacaacatgaatttcacgaagttgaagaacttcacgggtctgttcgagttctataagctgctgtgcgtgcgcggtatcatcaccagcaaaaccaaaagcctggacaaaggctacaacaaggcgctgaatgacctgtgcattaaggtaaacaattgggatctgttcttttcgccatccgaagataattttaccaacgacctgaacaagggtgaagaaatcaccagcgatacgaatattgaagcagcggaagagaatatcagcctggatctgatccagcagtactatctgacctttaacttcgacaatgaaccggagaacattagcattgagaatctgagcagcgacattatcggtcagctggaactgatgccgaatatcgaacgtttcccgaacggcaaaaagtacgagctggacaagtacactatgttccattacctgcgtgcacaggagtttgaacacggtaaaagccgtatcgcgctgaccaacagcgttaacgaggccctgctgaacccgagccgtgtctataccttcttcagcagcgactatgttaagaaagtgaacaaagccactgaggccgcgatgttcctgggctgggtggaacagctggtatatgacttcacggacgagacgagcgaagtgagcactaccgacaaaattgctgatattaccatcattatcccgtatattggtccggcactgaacattggcaacatgctgtacaaagacgattttgtgggtgccctgatcttctccggtgccgtgattctgctggagttcattccggagattgcgatcccggtgttgggtaccttcgcgctggtgtcctacatcgcgaataaggttctgacggttcagaccatcgataacgcgctgtcgaaacgtaatgaaaaatgggacgaggtttacaaatacattgttacgaattggctggcgaaagtcaatacccagatcgacctgatccgtaagaaaatgaaagaggcgctggagaatcaggcggaggccaccaaagcaattatcaactaccaatacaaccagtacacggaagaagagaagaataacattaacttcaatatcgatgatttgagcagcaagctgaatgaatctatcaacaaagcgatgatcaatatcaacaagtttttgaatcagtgtagcgtttcgtacctgatgaatagcatgattccgtatggcgtcaaacgtctggaggacttcgacgccagcctgaaagatgcgttgctgaaatacatttacgacaatcgtggtacgctgattggccaagttgaccgcttgaaagacaaagttaacaataccctgagcaccgacatcccatttcaactgagcaagtatgttgataatcaacgtctgttgagcactttcaccgagtatatcaaaaacatcatcaatactagcattctgaacctgcgttacgagagcaagcatctgattgatctgagccgttatgctagcaagatcaacatcggtagcaaggtcaattttgacccgatcgataagaaccagatccagctgtttaatctggaatcgagcaaaattgaggttatcctgaaaaaggccattgtctacaactccatgtacgagaatttctccaccagcttctggattcgcatcccgaaatacttcaacaagattagcctgaacaacgagtatactatcatcaactgtatggagaacaacagcggttggaaggtgtctctgaactatggtgagatcatttggaccttgcaggacaccaaagagatcaagcagcgcgtcgtgttcaagtactctcaaatgatcaacatttccgattacattaatcgttggatcttcgtgaccattacgaataaccgtctgaataagagcaagatttacatcaatggtcgcttgatcgatcagaaaccgattagcaacctgggtaatatccacgcaagcaacaagattatgttcaaattggacggttgccgcgatacccatcgttatatctggatcaagtatttcaacctgtttgataaagaactgaatgagaaggagatcaaagatttgtatgacaaccaatctaacagcggcattttgaaggacttctggggcgattatctgcaatacgataagccgtactatatgctgaacctgtatgatccgaacaaatatgtggatgtcaataatgtgggtattcgtggttacatgtatttgaagggtccgcgtggcagcgttatgacgaccaacatttacctgaactctagcctgtaccgtggtacgaaattcatcattaagaaatatgccagcggcaacaaagataacattgtgcgtaataacgatcgtgtctacatcaacgtggtcgtgaagaataaagagtaccgtctggcgaccaacgcttcgcaggcgggtgttgagaaaattctgagcgcgttggagatccctgatgtcggtaatctgagccaagtcgtggttatgaagagcaagaacgacaagggtatcactaacaagtgcaagatgaacctgcaagacaacaatggtaacgacatcggctttattggtttccaccagttcaacaatattgctaaactggtagcgagcaattggtacaatcgtcagattgagcgcagcagccgtactttgggctgtagctgggagtttatcccggtcgatgatggttggggcgaacgtccgctg

SEQ ID NO: 15 - protein, 1296 aa, Artificial Sequence
  Polypeptide Sequence of Modified BoNT/A “Cat-A”
MPFVNKQFNYKDPVNGVDIAYIKIPNAGQMQPVKAFKIHNKIWVIPERDTFTNPEEGDLNPPPEAKQVPVSYYDSTYLSTDNEKDNYLKGVTKLFERIYSTDLGRMLLTSIVRGIPFWGGSTIDTELKVIDTNCINVIQPDGSYRSEELNLVIIGPSADIIQFECKSFGHEVLNLTRNGYGSTQYIRFSPDFTFGFEESLEVDTNPLLGAGKFATDPAVTLAHELIHAGHRLYGIAINPNRVFKVNTNAYYEMSGLEVSFEELRTFGGHDAKFIDSLQENEFRLYYYNKFKDIASTLNKAKSIVGTTASLQYMKNVFKEKYLLSEDTSGKFSVDKLKFDKLYKMLTEIYTEDNFVKFFKVLNRKTYLNFDKAVFKINIVPKVNYTIYDGFNLRNTNLAANFNGQNTEINNMNFTKLKNFTGLFEFYKLLCVRGIITSKTKSLDKGYNKALNDLCIKVNNWDLFFSPSEDNFTNDLNKGEEITSDTNIEAAEENISLDLIQQYYLTFNFDNEPENISIENLSSDIIGQLELMPNIERFPNGKKYELDKYTMFHYLRAQEFEHGKSRIALTNSVNEALLNPSRVYTFFSSDYVKKVNKATEAAMFLGWVEQLVYDFTDETSEVSTTDKIADITIIIPYIGPALNIGNMLYKDDFVGALIFSGAVILLEFIPEIAIPVLGTFALVSYIANKVLTVQTIDNALSKRNEKWDEVYKYIVTNWLAKVNTQIDLIRKKMKEALENQAEATKAIINYQYNQYTEEEKNNINFNIDDLSSKLNESINKAMININKFLNQCSVSYLMNSMIPYGVKRLEDFDASLKDALLKYIYDNRGTLIGQVDRLKDKVNNTLSTDIPFQLSKYVDNQRLLSTFTEYIKNIINTSILNLRYESKHLIDLSRYASKINIGSKVNFDPIDKNQIQLFNLESSKIEVILKKAIVYNSMYENFSTSFWIRIPKYFNKISLNNEYTIINCMENNSGWKVSLNYGEIIWTLQDTKEIKQRVVFKYSQMINISDYINRWIFVTITNNRLNKSKIYINGRLIDQKPISNLGNIHASNKIMFKLDGCRDTHRYIWIKYFNLFDKELNEKEIKDLYDNQSNSGILKDFWGDYLQYDKPYYMLNLYDPNKYVDVNNVGIRGYMYLKGPRGSVMTTNIYLNSSLYRGTKFIIKKYASGNKDNIVRNNDRVYINVVVKNKEYRLATNASQAGVEKILSALEIPDVGNLSQVVVMKSKNDKGITNKCKMNLQDNNGNDIGFIGFHQFNNIAKLVASNWYNRQIERSSRTLGCSWEFIPVDDGWGERPL

SEQ ID NO: 16 - DNA, 3888 bp, Artificial Sequence
  Nucleotide Sequence of Modified BoNT/A “Cat-B”
atgccattcgtcaacaagcaattcaactacaaagacccagtcaacggcgtcgacatcgcatacatcaagattccgaacgccggtcaaatgcagccggttaaggcttttaagatccacaacaagatttgggttatcccggagcgtgacaccttcacgaacccggaagaaggcgatctgaacccgccaccggaagcgaagcaagtccctgtcagctactacgattcgacgtacctgagcacggataacgaaaaagataactacctgaaaggtgtgaccaagctgttcgaacgtatctacagcacggatctgggtcgcatgctgctgactagcattgttcgcggtatcccgttctggggtggtagcacgattgacaccgaactgaaggttatcgacactaactgcattaacgttattcaaccggatggtagctatcgtagcgaagagctgaatctggtcatcattggcccgagcgcagacattatccaattcgagtgcaagagctttggtcacgaggttctgaatctgacccgcaatggctatggtagcacccagtacattcgtttttcgccggattttaccttcggctttgaagagagcctggaggttgataccaatccgttgctgggtgcgggcaaattcgctaccgatccggctgtcacgctggcccatgaactgatccacgcaggccaccgcctgtacggcattgccatcaacccaaaccgtgtgttcaaggttaatacgaatgcatactacgagatgagcggcctggaagtcagcttcgaagaactgcgcaccttcggtggccatgacgctaaattcattgacagcttgcaagagaatgagttccgtctgtactactataacaaattcaaagacattgcaagcacgttgaacaaggccaaaagcatcgttggtactaccgcgtcgttgcagtatatgaagaatgtgtttaaagagaagtacctgctgtccgaggatacctccggcaagtttagcgttgataagctgaagtttgacaaactgtacaagatgctgaccgagatttacaccgaggacaactttgtgaaattcttcaaagtgttgaatcgtaaaacctatctgaattttgacaaagcggttttcaagattaacatcgtgccgaaggtgaactacaccatctatgacggttttaacctgcgtaacaccaacctggcggcgaactttaacggtcagaatacggaaatcaacaacatgaatttcacgaagttgaagaacttcacgggtctgttcgagttctataagctgctgtgcgtgcgcggtatcatcaccagcaaaaccaaaagcctggacaaaggctacaacaaggcgctgaatgacctgtgcattaaggtaaacaattgggatctgttcttttcgccatccgaagataattttaccaacgacctgaacaagggtgaagaaatcaccagcgatacgaatattgaagcagcggaagagaatatcagcctggatctgatccagcagtactatctgacctttaacttcgacaatgaaccggagaacattagcattgagaatctgagcagcgacattatcggtcagctggaactgatgccgaatatcgaacgtttcccgaacggcaaaaagtacgagctggacaagtacactatgttccattacctgcgtgcacaggagtttgaacacggtaaaagccgtatcgcgctgaccaacagcgttaacgaggccctgctgaacccgagccgtgtctataccttcttcagcagcgactatgttaagaaagtgaacaaagccactgaggccgcgatgttcctgggctgggtggaacagctggtatatgacttcacggacgagacgagcgaagtgagcactaccgacaaaattgctgatattaccatcattatcccgtatattggtccggcactgaacattggcaacatgctgtacaaagacgattttgtgggtgccctgatcttctccggtgccgtgattctgctggagttcattccggagattgcgatcccggtgttgggtaccttcgcgctggtgtcctacatcgcgaataaggttctgacggttcagaccatcgataacgcgctgtcgaaacgtaatgaaaaatgggacgaggtttacaaatacattgttacgaattggctggcgaaagtcaatacccagatcgacctgatccgtaagaaaatgaaagaggcgctggagaatcaggcggaggccaccaaagcaattatcaactaccaatacaaccagtacacggaagaagagaagaataacattaacttcaatatcgatgatttgagcagcaagctgaatgaatctatcaacaaagcgatgatcaatatcaacaagtttttgaatcagtgtagcgtttcgtacctgatgaatagcatgattccgtatggcgtcaaacgtctggaggacttcgacgccagcctgaaagatgcgttgctgaaatacatttacgacaatcgtggtacgctgattggccaagttgaccgcttgaaagacaaagttaacaataccctgagcaccgacatcccatttcaactgagcaagtatgttgataatcaacgtctgttgagcactttcaccgagtatatcaaaaacatcatcaatactagcattctgaacctgcgttacgagagcaatcatctgattgatctgagccgttatgctagcaagatcaacatcggtagcaaggtcaattttgacccgatcgataagaaccagatccagctgtttaatctggaatcgagcaaaattgaggttatcctgaaaaaggccattgtctacaactccatgtacgagaatttctccaccagcttctggattcgcatcccgaaatacttcaagaagattagcctgaacaacgagtatactatcatcaactgtatggagaacaacagcggttggaaggtgtctctgaactatggtgagatcatttggaccttgcaggacaccaaagagatcaagcagcgcgtcgtgttcaagtactctcaaatgatcaacatttccgattacattaatcgttggatcttcgtgaccattacgaataaccgtctgaataagagcaagatttacatcaatggtcgcttgatcgatcagaaaccgattagcaacctgggtaatatccacgcaagcaacaagattatgttcaaattggacggttgccgcgatacccatcgttatatctggatcaagtatttcaacctgtttgataaagaactgaatgagaaggagatcaaagatttgtatgacaaccaatctaacagcggcattttgaaggacttctggggcgattatctgcaatacgataagccgtactatatgctgaacctgtatgatccgaacaaatatgtggatgtcaataatgtgggtattcgtggttacatgtatttgaagggtccgcgtggcagcgttatgacgaccaacatttacctgaactctagcctgtaccgtggtacgaaattcatcattaagaaatatgccagcggcaacaaagataacattgtgcgtaataacgatcgtgtctacatcaacgtggtcgtgaagaataaagagtaccgtctggcgaccaacgcttcgcaggcgggtgttgagaaaattctgagcgcgttggagatccctgatgtcggtaatctgagccaagtcgtggttatgaagagcaagaacgacaagggtatcactaacaagtgcaagatgaacctgcaagacaacaatggtaacgacatcggctttattggtttccaccagttcaacaatattgctaaactggtagcgagcaattggtacaatcgtcagattgagcgcagcagccgtactttgggctgtagctgggagtttatcccggtcgatgatggttggggcgaacgtccgctg

SEQ ID NO: 17 - protein, 1296 aa, Artificial Sequence
  Polypeptide Sequence of Modified BoNT/A “Cat-B”
MPFVNKQFNYKDPVNGVDIAYIKIPNAGQMQPVKAFKIHNKIWVIPERDTFTNPEEGDLNPPPEAKQVPVSYYDSTYLSTDNEKDNYLKGVTKLFERIYSTDLGRMLLTSIVRGIPFWGGSTIDTELKVIDTNCINVIQPDGSYRSEELNLVIIGPSADIIQFECKSFGHEVLNLTRNGYGSTQYIRFSPDFTFGFEESLEVDTNPLLGAGKFATDPAVTLAHELIHAGHRLYGIAINPNRVFKVNTNAYYEMSGLEVSFEELRTFGGHDAKFIDSLQENEFRLYYYNKFKDIASTLNKAKSIVGTTASLQYMKNVFKEKYLLSEDTSGKFSVDKLKFDKLYKMLTEIYTEDNFVKFFKVLNRKTYLNFDKAVFKINIVPKVNYTIYDGFNLRNTNLAANFNGQNTEINNMNFTKLKNFTGLFEFYKLLCVRGIITSKTKSLDKGYNKALNDLCIKVNNWDLFFSPSEDNFTNDLNKGEEITSDTNIEAAEENISLDLIQQYYLTFNFDNEPENISIENLSSDIIGQLELMPNIERFPNGKKYELDKYTMFHYLRAQEFEHGKSRIALTNSVNEALLNPSRVYTFFSSDYVKKVNKATEAAMFLGWVEQLVYDFTDETSEVSTTDKIADITIIIPYIGPALNIGNMLYKDDFVGALIFSGAVILLEFIPEIAIPVLGTFALVSYIANKVLTVQTIDNALSKRNEKWDEVYKYIVTNWLAKVNTQIDLIRKKMKEALENQAEATKAIINYQYNQYTEEEKNNINFNIDDLSSKLNESINKAMININKFLNQCSVSYLMNSMIPYGVKRLEDFDASLKDALLKYIYDNRGTLIGQVDRLKDKVNNTLSTDIPFQLSKYVDNQRLLSTFTEYIKNIINTSILNLRYESNHLIDLSRYASKINIGSKVNFDPIDKNQIQLFNLESSKIEVILKKAIVYNSMYENFSTSFWIRIPKYFKKISLNNEYTIINCMENNSGWKVSLNYGEIIWTLQDTKEIKQRVVFKYSQMINISDYINRWIFVTITNNRLNKSKIYINGRLIDQKPISNLGNIHASNKIMFKLDGCRDTHRYIWIKYFNLFDKELNEKEIKDLYDNQSNSGILKDFWGDYLQYDKPYYMLNLYDPNKYVDVNNVGIRGYMYLKGPRGSVMTTNIYLNSSLYRGTKFIIKKYASGNKDNIVRNNDRVYINVVVKNKEYRLATNASQAGVEKILSALEIPDVGNLSQVVVMKSKNDKGITNKCKMNLQDNNGNDIGFIGFHQFNNIAKLVASNWYNRQIERSSRTLGCSWEFIPVDDGWGERPL

SEQ ID NO: 18 - DNA, 3888 bp, Artificial Sequence
  Nucleotide Sequence of Modified BoNT/A “Cat-C”
atgccattcgtcaacaagcaattcaactacaaagacccagtcaacggcgtcgacatcgcatacatcaagattccgaacgccggtcaaatgcagccggttaaggcttttaagatccacaacaagatttgggttatcccggagcgtgacaccttcacgaacccggaagaaggcgatctgaacccgccaccggaagcgaagcaagtccctgtcagctactacgattcgacgtacctgagcacggataacgaaaaagataactacctgaaaggtgtgaccaagctgttcgaacgtatctacagcacggatctgggtcgcatgctgctgactagcattgttcgcggtatcccgttctggggtggtagcacgattgacaccgaactgaaggttatcgacactaactgcattaacgttattcaaccggatggtagctatcgtagcgaagagctgaatctggtcatcattggcccgagcgcagacattatccaattcgagtgcaagagctttggtcacgaggttctgaatctgacccgcaatggctatggtagcacccagtacattcgtttttcgccggattttaccttcggctttgaagagagcctggaggttgataccaatccgttgctgggtgcgggcaaattcgctaccgatccggctgtcacgctggcccatgaactgatccacgcaggccaccgcctgtacggcattgccatcaacccaaaccgtgtgttcaaggttaatacgaatgcatactacgagatgagcggcctggaagtcagcttcgaagaactgcgcaccttcggtggccatgacgctaaattcattgacagcttgcaagagaatgagttccgtctgtactactataacaaattcaaagacattgcaagcacgttgaacaaggccaaaagcatcgttggtactaccgcgtcgttgcagtatatgaagaatgtgtttaaagagaagtacctgctgtccgaggatacctccggcaagtttagcgttgataagctgaagtttgacaaactgtacaagatgctgaccgagatttacaccgaggacaactttgtgaaattcttcaaagtgttgaatcgtaaaacctatctgaattttgacaaagcggttttcaagattaacatcgtgccgaaggtgaactacaccatctatgacggttttaacctgcgtaacaccaacctggcggcgaactttaacggtcagaatacggaaatcaacaacatgaatttcacgaagttgaagaacttcacgggtctgttcgagttctataagctgctgtgcgtgcgcggtatcatcaccagcaaaaccaaaagcctggacaaaggctacaacaaggcgctgaatgacctgtgcattaaggtaaacaattgggatctgttcttttcgccatccgaagataattttaccaacgacctgaacaagggtgaagaaatcaccagcgatacgaatattgaagcagcggaagagaatatcagcctggatctgatccagcagtactatctgacctttaacttcgacaatgaaccggagaacattagcattgagaatctgagcagcgacattatcggtcagctggaactgatgccgaatatcgaacgtttcccgaacggcaaaaagtacgagctggacaagtacactatgttccattacctgcgtgcacaggagtttgaacacggtaaaagccgtatcgcgctgaccaacagcgttaacgaggccctgctgaacccgagccgtgtctataccttcttcagcagcgactatgttaagaaagtgaacaaagccactgaggccgcgatgttcctgggctgggtggaacagctggtatatgacttcacggacgagacgagcgaagtgagcactaccgacaaaattgctgatattaccatcattatcccgtatattggtccggcactgaacattggcaacatgctgtacaaagacgattttgtgggtgccctgatcttctccggtgccgtgattctgctggagttcattccggagattgcgatcccggtgttgggtaccttcgcgctggtgtcctacatcgcgaataaggttctgacggttcagaccatcgataacgcgctgtcgaaacgtaatgaaaaatgggacgaggtttacaaatacattgttacgaattggctggcgaaagtcaatacccagatcgacctgatccgtaagaaaatgaaagaggcgctggagaatcaggcggaggccaccaaagcaattatcaactaccaatacaaccagtacacggaagaagagaagaataacattaacttcaatatcgatgatttgagcagcaagctgaatgaatctatcaacaaagcgatgatcaatatcaacaagtttttgaatcagtgtagcgtttcgtacctgatgaatagcatgattccgtatggcgtcaaacgtctggaggacttcgacgccagcctgaaagatgcgttgctgaaatacatttacgacaatcgtggtacgctgattggccaagttgaccgcttgaaagacaaagttaacaataccctgagcaccgacatcccatttcaactgagcaagtatgttgataatcaacgtctgttgagcactttcaccgagtatatcaaaaacatcatcaatactagcattctgaacctgcgttacgagagcaatcatctgattgatctgagccgttatgctagcaagatcaacatcggtagcaaggtcaattttgacccgatcgataagaaccagatccagctgtttaatctggaatcgagcaaaattgaggttatcctgaaaaaggccattgtctacaactccatgtacgagaatttctccaccagcttctggattcgcatcccgaaatacttcaacaagattagcctgaacaacgagtatactatcatcaactgtatggagaacaacagcggttggaaggtgtctctgaactatggtgagatcatttggaccttgcaggacaccaaagagatcaagcagcgcgtcgtgttcaagtactctcaaatgatcaacatttccgattacattaatcgttggatcttcgtgaccattacgaataaccgtctgaagaagagcaagatttacatcaatggtcgcttgatcgatcagaaaccgattagcaacctgggtaatatccacgcaagcaacaagattatgttcaaattggacggttgccgcgatacccatcgttatatctggatcaagtatttcaacctgtttgataaagaactgaatgagaaggagatcaaagatttgtatgacaaccaatctaacagcggcattttgaaggacttctggggcgattatctgcaatacgataagccgtactatatgctgaacctgtatgatccgaacaaatatgtggatgtcaataatgtgggtattcgtggttacatgtatttgaagggtccgcgtggcagcgttatgacgaccaacatttacctgaactctagcctgtaccgtggtacgaaattcatcattaagaaatatgccagcggcaacaaagataacattgtgcgtaataacgatcgtgtctacatcaacgtggtcgtgaagaataaagagtaccgtctggcgaccaacgcttcgcaggcgggtgttgagaaaattctgagcgcgttggagatccctgatgtcggtaatctgagccaagtcgtggttatgaagagcaagaacgacaagggtatcactaacaagtgcaagatgaacctgcaagacaacaatggtaacgacatcggctttattggtttccaccagttcaacaatattgctaaactggtagcgagcaattggtacaatcgtcagattgagcgcagcagccgtactttgggctgtagctgggagtttatcccggtcgatgatggttggggcgaacgtccgctg

SEQ ID NO: 19 - protein, 1296 aa, Artificial Sequence
  Polypeptide Sequence of Modified BoNT/A “Cat-C”
MPFVNKQFNYKDPVNGVDIAYIKIPNAGQMQPVKAFKIHNKIWVIPERDTFTNPEEGDLNPPPEAKQVPVSYYDSTYLSTDNEKDNYLKGVTKLFERIYSTDLGRMLLTSIVRGIPFWGGSTIDTELKVIDTNCINVIQPDGSYRSEELNLVIIGPSADIIQFECKSFGHEVLNLTRNGYGSTQYIRFSPDFTFGFEESLEVDTNPLLGAGKFATDPAVTLAHELIHAGHRLYGIAINPNRVFKVNTNAYYEMSGLEVSFEELRTFGGHDAKFIDSLQENEFRLYYYNKFKDIASTLNKAKSIVGTTASLQYMKNVFKEKYLLSEDTSGKFSVDKLKFDKLYKMLTEIYTEDNFVKFFKVLNRKTYLNFDKAVFKINIVPKVNYTIYDGFNLRNTNLAANFNGQNTEINNMNFTKLKNFTGLFEFYKLLCVRGIITSKTKSLDKGYNKALNDLCIKVNNWDLFFSPSEDNFTNDLNKGEEITSDTNIEAAEENISLDLIQQYYLTFNFDNEPENISIENLSSDIIGQLELMPNIERFPNGKKYELDKYTMFHYLRAQEFEHGKSRIALTNSVNEALLNPSRVYTFFSSDYVKKVNKATEAAMFLGWVEQLVYDFTDETSEVSTTDKIADITIIIPYIGPALNIGNMLYKDDFVGALIFSGAVILLEFIPEIAIPVLGTFALVSYIANKVLTVQTIDNALSKRNEKWDEVYKYIVTNWLAKVNTQIDLIRKKMKEALENQAEATKAIINYQYNQYTEEEKNNINFNIDDLSSKLNESINKAMININKFLNQCSVSYLMNSMIPYGVKRLEDFDASLKDALLKYIYDNRGTLIGQVDRLKDKVNNTLSTDIPFQLSKYVDNQRLLSTFTEYIKNIINTSILNLRYESNHLIDLSRYASKINIGSKVNFDPIDKNQIQLFNLESSKIEVILKKAIVYNSMYENFSTSFWIRIPKYFNKISLNNEYTIINCMENNSGWKVSLNYGEIIWTLQDTKEIKQRVVFKYSQMINISDYINRWIFVTITNNRLKKSKIYINGRLIDQKPISNLGNIHASNKIMFKLDGCRDTHRYIWIKYFNLFDKELNEKEIKDLYDNQSNSGILKDFWGDYLQYDKPYYMLNLYDPNKYVDVNNVGIRGYMYLKGPRGSVMTTNIYLNSSLYRGTKFIIKKYASGNKDNIVRNNDRVYINVVVKNKEYRLATNASQAGVEKILSALEIPDVGNLSQVVVMKSKNDKGITNKCKMNLQDNNGNDIGFIGFHQFNNIAKLVASNWYNRQIERSSRTLGCSWEFIPVDDGWGERPL

SEQ ID NO: 20 - DNA, 3888 bp, Artificial Sequence
  Nucleotide Sequence of Modified BoNT/A “Cat-D”
atgccattcgtcaacaagcaattcaactacaaagacccagtcaacggcgtcgacatcgcatacatcaagattccgaacgccggtcaaatgcagccggttaaggcttttaagatccacaacaagatttgggttatcccggagcgtgacaccttcacgaacccggaagaaggcgatctgaacccgccaccggaagcgaagcaagtccctgtcagctactacgattcgacgtacctgagcacggataacgaaaaagataactacctgaaaggtgtgaccaagctgttcgaacgtatctacagcacggatctgggtcgcatgctgctgactagcattgttcgcggtatcccgttctggggtggtagcacgattgacaccgaactgaaggttatcgacactaactgcattaacgttattcaaccggatggtagctatcgtagcgaagagctgaatctggtcatcattggcccgagcgcagacattatccaattcgagtgcaagagctttggtcacgaggttctgaatctgacccgcaatggctatggtagcacccagtacattcgtttttcgccggattttaccttcggctttgaagagagcctggaggttgataccaatccgttgctgggtgcgggcaaattcgctaccgatccggctgtcacgctggcccatgaactgatccacgcaggccaccgcctgtacggcattgccatcaacccaaaccgtgtgttcaaggttaatacgaatgcatactacgagatgagcggcctggaagtcagcttcgaagaactgcgcaccttcggtggccatgacgctaaattcattgacagcttgcaagagaatgagttccgtctgtactactataacaaattcaaagacattgcaagcacgttgaacaaggccaaaagcatcgttggtactaccgcgtcgttgcagtatatgaagaatgtgtttaaagagaagtacctgctgtccgaggatacctccggcaagtttagcgttgataagctgaagtttgacaaactgtacaagatgctgaccgagatttacaccgaggacaactttgtgaaattcttcaaagtgttgaatcgtaaaacctatctgaattttgacaaagcggttttcaagattaacatcgtgccgaaggtgaactacaccatctatgacggttttaacctgcgtaacaccaacctggcggcgaactttaacggtcagaatacggaaatcaacaacatgaatttcacgaagttgaagaacttcacgggtctgttcgagttctataagctgctgtgcgtgcgcggtatcatcaccagcaaaaccaaaagcctggacaaaggctacaacaaggcgctgaatgacctgtgcattaaggtaaacaattgggatctgttcttttcgccatccgaagataattttaccaacgacctgaacaagggtgaagaaatcaccagcgatacgaatattgaagcagcggaagagaatatcagcctggatctgatccagcagtactatctgacctttaacttcgacaatgaaccggagaacattagcattgagaatctgagcagcgacattatcggtcagctggaactgatgccgaatatcgaacgtttcccgaacggcaaaaagtacgagctggacaagtacactatgttccattacctgcgtgcacaggagtttgaacacggtaaaagccgtatcgcgctgaccaacagcgttaacgaggccctgctgaacccgagccgtgtctataccttcttcagcagcgactatgttaagaaagtgaacaaagccactgaggccgcgatgttcctgggctgggtggaacagctggtatatgacttcacggacgagacgagcgaagtgagcactaccgacaaaattgctgatattaccatcattatcccgtatattggtccggcactgaacattggcaacatgctgtacaaagacgattttgtgggtgccctgatcttctccggtgccgtgattctgctggagttcattccggagattgcgatcccggtgttgggtaccttcgcgctggtgtcctacatcgcgaataaggttctgacggttcagaccatcgataacgcgctgtcgaaacgtaatgaaaaatgggacgaggtttacaaatacattgttacgaattggctggcgaaagtcaatacccagatcgacctgatccgtaagaaaatgaaagaggcgctggagaatcaggcggaggccaccaaagcaattatcaactaccaatacaaccagtacacggaagaagagaagaataacattaacttcaatatcgatgatttgagcagcaagctgaatgaatctatcaacaaagcgatgatcaatatcaacaagtttttgaatcagtgtagcgtttcgtacctgatgaatagcatgattccgtatggcgtcaaacgtctggaggacttcgacgccagcctgaaagatgcgttgctgaaatacatttacgacaatcgtggtacgctgattggccaagttgaccgcttgaaagacaaagttaacaataccctgagcaccgacatcccatttcaactgagcaagtatgttgataatcaacgtctgttgagcactttcaccgagtatatcaaaaacatcatcaatactagcattctgaacctgcgttacgagagcaatcatctgattgatctgagccgttatgcaagcaagatcaacatcggtagcaaggtcaattttgacccgatcgataagaaccagatccagctgtttaatctggaatcgagcaaaattgaggttatcctgaaaaacgccattgtctacaactccatgtacgagaatttctccaccagcttctggattcgcatcccgaaatacttcaacagcattagcctgaacaacgagtatactatcatcaactgtatggagaacaacagcggttggaaggtgtctctgaactatggtgagatcatttggaccttgcaggacacccaagagatcaagcagcgcgtcgtgttcaagtactctcaaatgatcaacatttccgattacattaatcgttggatcttcgtgaccattacgaataaccgtctgaataacagcaagatttacatcaatggtcgcttgatcgatcagaaaccgattagcaacctgggtaatatccacgcaagcaacaacattatgttcaaattggacggttgccgcgatacccatcgttatatctggatcaagtatttcaacctgtttgataaagaactgaatgagaaggagatcaaagatttgtatgacaaccaatctaacagcggcattttgaaggacttctggggcgattatctgcaatacgataagccgtactatatgctgaacctgtatgatccgaacaaatatgtggatgtcaataatgtgggtattcgtggttacatgtatttgaagggtccgcgtggcagcgttatgacgaccaacatttacctgaactctagcctgtaccgtggtacgaaattcatcattaagaaatatgccagcggcaacaaagataacattgtgcgtaataacgatcgtgtctacatcaacgtggtcgtgaagcgtaaagagtaccgtctggcgaccaacgcttcgcaggcgggtgttgagaaaattctgagcgcgttggagatccctcgtgtccgtcgtctgagccaagtcgtggttatgaagagcaagaacgaccagggtatcactaacaagtgcaagatgaacctgcaagaccgtcgtggtaacgacatcggctttattggtttccaccagttcaacaatattgctaaactggtagcgagcaattggtacaatcgtcagattgagcgccgtagccgtcgtttgggctgtagctgggagtttatcccggtcgatgatggttggggcgaacgtccgctg

SEQ ID NO: 21 - protein, 1296 aa, Artificial Sequence
  Polypeptide Sequence of Modified BoNT/A “Cat-D”
MPFVNKQFNYKDPVNGVDIAYIKIPNAGQMQPVKAFKIHNKIWVIPERDTFTNPEEGDLNPPPEAKQVPVSYYDSTYLSTDNEKDNYLKGVTKLFERIYSTDLGRMLLTSIVRGIPFWGGSTIDTELKVIDTNCINVIQPDGSYRSEELNLVIIGPSADIIQFECKSFGHEVLNLTRNGYGSTQYIRFSPDFTFGFEESLEVDTNPLLGAGKFATDPAVTLAHELIHAGHRLYGIAINPNRVFKVNTNAYYEMSGLEVSFEELRTFGGHDAKFIDSLQENEFRLYYYNKFKDIASTLNKAKSIVGTTASLQYMKNVFKEKYLLSEDTSGKFSVDKLKFDKLYKMLTEIYTEDNFVKFFKVLNRKTYLNFDKAVFKINIVPKVNYTIYDGFNLRNTNLAANFNGQNTEINNMNFTKLKNFTGLFEFYKLLCVRGIITSKTKSLDKGYNKALNDLCIKVNNWDLFFSPSEDNFTNDLNKGEEITSDTNIEAAEENISLDLIQQYYLTFNFDNEPENISIENLSSDIIGQLELMPNIERFPNGKKYELDKYTMFHYLRAQEFEHGKSRIALTNSVNEALLNPSRVYTFFSSDYVKKVNKATEAAMFLGWVEQLVYDFTDETSEVSTTDKIADITIIIPYIGPALNIGNMLYKDDFVGALIFSGAVILLEFIPEIAIPVLGTFALVSYIANKVLTVQTIDNALSKRNEKWDEVYKYIVTNWLAKVNTQIDLIRKKMKEALENQAEATKAIINYQYNQYTEEEKNNINFNIDDLSSKLNESINKAMININKFLNQCSVSYLMNSMIPYGVKRLEDFDASLKDALLKYIYDNRGTLIGQVDRLKDKVNNTLSTDIPFQLSKYVDNQRLLSTFTEYIKNIINTSILNLRYESNHLIDLSRYASKINIGSKVNFDPIDKNQIQLFNLESSKIEVILKNAIVYNSMYENFSTSFWIRIPKYFNSISLNNEYTIINCMENNSGWKVSLNYGEIIWTLQDTQEIKQRVVFKYSQMINISDYINRWIFVTITNNRLNNSKIYINGRLIDQKPISNLGNIHASNNIMFKLDGCRDTHRYIWIKYFNLFDKELNEKEIKDLYDNQSNSGILKDFWGDYLQYDKPYYMLNLYDPNKYVDVNNVGIRGYMYLKGPRGSVMTTNIYLNSSLYRGTKFIIKKYASGNKDNIVRNNDRVYINVVVKRKEYRLATNASQAGVEKILSALEIPRVRRLSQVVVMKSKNDQGITNKCKMNLQDRRGNDIGFIGFHQFNNIAKLVASNWYNRQIERRSRRLGCSWEFIPVDDGWGERPL